(19) United States
(12) Patent Application Publication
(10) Pub. No.: US 2024/0415946 A1
(43) Pub. Date: Dec. 19, 2024

(54) THERAPEUTIC RNA FOR LUNG CANCER

(71) Applicants: BIONTECH SE, Mainz (DE); TRON-TRANSLATIONALE ONKOLOGIE AN DER UNIVERSITÄTSMEDIZIN DER JOHANNES GUTENBE, Mainz (DE)

(72) Inventors: Ugur SAHIN, Mainz (DE); David WEBER, Wiesbaden (DE); Carina WALTER, Mainz (DE); Diana BAREA ROLDÁN, Alsheim (DE); Ruprecht KUNER, Ostfildern (DE); Melanie HEIN, Mainz (DE); Martin SUCHAN,, Mainz (DE); Vetena KISSLER, Undenheim (DE); Lena Mareen KRANZ, Hofheim am Taunus (DE)

(21) Appl. No.: 18/699,937

(22) PCT Filed: Oct. 10, 2022
(86) PCT No.: PCT/EP2022/078084

§ 371 Date: Apr. 10, 2024

(30) Foreign Application Priority Data
Oct. 11, 2021 (WO) PCT/EP2021/078022
Jan. 5, 2022 (WO) PCT/EP2022/050135

(51) Int. Cl. A61K 39/00; A61K 9/00; A61K 9/127; A61P 35/00

(52) U.S. Cl. CPC A61K 39/001186; A61K 9/0019; A61K 9/127; A61K 39/001102; A61K 39/001189; A61P 35/00; A61K 2039/53; A61K 2039/54; A61K 2039/55544; A61K 2039/55555; A61K 2039/572; A61K 2039/86

(57) ABSTRACT

This disclosure relates to the field of RNA to treat lung cancer, in particular non-small-cell lung carcinoma (NSCLC). Lung cancer is the third most frequent malignancy in women and the second most frequent malignancy in men. NSCLC accounts for about 85% of all lung cancers. Disclosed herein are compositions, uses, and methods for treatment of lung cancers. Administration of therapeutic RNAs to a patient having lung cancer disclosed herein can reduce tumor size, prolong time to progressive disease, and/or protect against metastasis and/or recurrence of the tumor and ultimately extend survival time.

This disclosure relates to the field of RNA to treat lung cancer, in particular non-small-cell lung carcinoma (NSCLC). Lung cancer is the third most frequent malignancy in women and the second most frequent malignancy in men. NSCLC accounts for about 85% of all lung cancers.

Disclosed herein are compositions, uses, and methods for treatment of lung cancers. Administration of therapeutic RNAs to a patient having lung cancer disclosed herein can reduce tumor size, prolong time to progressive disease, and/or protect against metastasis and/or recurrence of the tumor and ultimately extend survival time.

### SUMMARY

The present invention generally embraces the immunotherapeutic treatment of a subject comprising the administration of RNA, i.e., vaccine RNA, encoding a set of amino acid sequences, i.e., vaccine antigens, each of said amino acid sequences comprising a tumor antigen, an immunogenic variant thereof, or an immunogenic fragment of the tumor antigen or the immunogenic variant thereof, i.e., an antigenic peptide or protein. Thus, the vaccine antigen comprises an epitope of a tumor antigen for inducing an immune response against the tumor antigen in the subject. RNA encoding vaccine antigen is administered to provide (following expression of the polynucleotide by appropriate target cells) antigen for induction, i.e., stimulation, priming and/or expansion, of an immune response which is targeted to target antigen (tumor antigen) or a procession product thereof. In one embodiment, the immune response which is to be induced according to the present disclosure is a T cell-mediated immune response. In one embodiment, the immune response is an anti-cancer, in particular anti-lung cancer immune response such as an anti-non-small-cell lung carcinoma (NSCLC) immune response.

The vaccine described herein comprises as the active principle single-stranded RNA that may be translated into the respective protein upon entering cells of a recipient. In addition to wildtype or codon-optimized sequences encoding the antigen sequence, the RNA may contain one or more structural elements optimized for maximal efficacy of the RNA with respect to stability and translational efficiency (5′ cap, 5′ UTR, 3′ UTR, poly(A)-tail). In one embodiment, the RNA contains all of these elements. In one embodiment, beta-S-ARCA(D1) (m₂^{7,2′-O}GppSpG) may be utilized as specific capping structure at the 5′-end of the RNA drug substances. As 5′-UTR sequence, the 5′-UTR sequence of the human alpha-globin mRNA, optionally with an optimized ‘Kozak sequence’ to increase translational efficiency may be used. As 3′-UTR sequence, a combination of two sequence elements (FI element) derived from the “amino terminal enhancer of split” (AES) mRNA (called F) and the mitochondrial encoded 12S ribosomal RNA (called I) placed between the coding sequence and the poly(A)-tail to assure higher maximum protein levels and prolonged persistence of the mRNA may be used.

These were identified by an ex vivo selection process for sequences that confer RNA stability and augment total protein expression (see WO 2017/060314, herein incorporated by reference). Furthermore, a poly(A)-tail measuring 110 nucleotides in length, consisting of a stretch of 30 adenosine residues, followed by a 10 nucleotide linker sequence (of random nucleotides) and another 70 adenosine residues may be used. This poly(A)-tail sequence was designed to enhance RNA stability and translational efficiency.

In one embodiment, a vaccine antigen described herein comprises an amino acid sequence which breaks immunological tolerance. The amino acid sequence which breaks immunological tolerance may be fused to the C-terminus of the vaccine sequence, i.e., antigenic peptide or protein, either directly or separated by a linker. Optionally, the amino acid sequence which breaks immunological tolerance may link the antigenic peptide or protein and a MITD as further described below. The amino acid sequence which breaks immunological tolerance may be RNA encoded. In one embodiment, the antigen-targeting RNAs are applied together with RNA coding for an amino acid sequence which breaks immunological tolerance. This RNA coding for an amino acid sequence which breaks immunological tolerance may contain structural elements optimized for maximal efficacy of the RNA with respect to stability and translational efficiency (5′ cap, 5′ UTR, 3′ UTR, poly(A)-tail) described above for the antigen-encoding RNA.

In one embodiment, the amino acid sequence which breaks immunological tolerance comprises helper epitopes. In one embodiment, the helper epitopes may be tetanus toxoid-derived, e.g., P2P16 amino acid sequences derived from the tetanus toxoid (TT) of Clostridium tetani. These sequences may support to overcome self-tolerance mechanisms for efficient induction of immune responses to self-antigens by providing tumor-unspecific T-cell help during priming. The tetanus toxoid heavy chain includes epitopes that can bind promiscuously to MHC class II alleles and induce CD4⁺ memory T cells in almost all tetanus vaccinated individuals. In addition, the combination of TT helper epitopes with tumor-associated antigens is known to improve the immune stimulation compared to the application of tumor-associated antigen alone by providing CD4⁺ mediated T-cell help during priming. To reduce the risk of stimulating CD8⁺ T cells, two peptide sequences known to contain promiscuously binding helper epitopes may be used to ensure binding to as many MHC class II alleles as possible, e.g., P2 and P16.

Furthermore, see (secretory signal peptide) and/or MITD (MHC class I trafficking domain) may be fused to the antigen-encoding regions and/or helper epitope-encoding regions in a way that the respective elements are translated as N- or C-terminal tag, respectively. Fusion-protein tags derived from the sequence encoding the human MHC class I complex (HLA-B51, haplotype A2, B27/B51, Cw2/Cw3), have been shown to improve antigen processing and presentation. Sec may correspond to the 78 bp fragment coding for the secretory signal peptide, which guides translocation of the nascent polypeptide chain into the endoplasmatic reticulum. MITD may correspond to the transmembrane and cytoplasmic domain of the MHC class I molecule, also called MHC class I trafficking domain. Antigens such as CLDN6 having their own secretory signal peptide and transmembrane domain may not require addition of fusion tags. Sequences coding for short linker peptides predominantly consisting of the amino acids glycine (G) and serine (S), as commonly used for fusion proteins may be used as GS/Linkers.

The vaccine RNA may be complexed with liposomes to generate serum-stable RNA-lipoplexes (RNA(LIP)) for intravenous (i.v.) administration. If a combination of different RNAs is used, the RNAs may be separately complexed with liposomes to generate serum-stable RNA-lipoplexes (RNA(LIP)) for intravenous (i.v.) administration. RNA(LIP) targets antigen-presenting cells (APCs) in lymphoid organs which results in an efficient stimulation of the immune system.

The RNA lipoplex particles may be prepared using liposomes that may be obtained by injecting a solution of lipids in ethanol into water or a suitable aqueous phase. In one embodiment, the aqueous phase has an acidic pH. In one embodiment, the aqueous phase comprises acetic acid, e.g., in an amount of about 5 mM. Liposomes may be used for preparing RNA lipoplex particles by mixing the liposomes with RNA. In one embodiment, the liposomes and RNA lipoplex particles comprise at least one cationic lipid and at least one additional lipid. In one embodiment, the at least one cationic lipid comprises 1,2-di-O-octadecenyl-3-trimethylammonium propane (DOTMA). In one embodiment, the at least one additional lipid comprises 1,2-di-(9Z-octadecenoyl)-sn-glycero-3-phosphoethanolamine (DOPE). In one embodiment, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In one embodiment, the liposomes and RNA lipoplex particles comprise 1,2-di-O-octadecenyl-3-trimethylammonium propane (DOTMA) and 1,2-di-(9Z-octadecenoyl)-sn-glycero-3-phosphoethanolamine (DOPE). In one embodiment, the molar ratio of the at least one cationic lipid to the at least one additional lipid is about 2:1. In one embodiment, at physiological pH, the charge ratio of positive charges to negative charges in the RNA lipoplex particles is from about 1.6:2 to about 1:2, or about 1.6:2 to about 1.1:2. In specific embodiments, the charge ratio of positive charges to negative charges in the RNA lipoplex particles at physiological pH is about 1.6:2.0, about 1.5:2.0, about 1.4:2.0, about 1.3:2.0, about 1.2:2.0, about 1.1:2.0, or about 1:2.0. In one embodiment, vaccine RNA is co-formulated as lipoplex particles with an RNA encoding an amino acid sequence which breaks immunological tolerance.

In one aspect, the invention relates to a composition or medical preparation comprising at least one RNA, wherein the at least one RNA encodes the following amino acid sequences:
(i) an amino acid sequence comprising claudin 6 (CLDN6), an immunogenic variant thereof, or an immunogenic fragment of the CLDN6 or the immunogenic variant thereof;
(ii) an amino acid sequence comprising Kita-kyushu lung cancer antigen 1 (KK-LC-1), an immunogenic variant thereof, or an immunogenic fragment of the KK-LC-1 or the immunogenic variant thereof;
(iii) an amino acid sequence comprising Melanoma antigen A3 (MAGE-A3), an immunogenic variant thereof, or an immunogenic fragment of the MAGE-A3 or the immunogenic variant thereof;
(iv) an amino acid sequence comprising Melanoma antigen 4 (MAGE-A4), an immunogenic variant thereof, or an immunogenic fragment of the MAGE-A4 or the immunogenic variant thereof; and
(v) an amino acid sequence comprising Preferentially Expressed Antigen In Melanoma (PRAME), an immunogenic variant thereof, or an immunogenic fragment of the PRAME or the immunogenic variant thereof.

In one embodiment, the at least one RNA further encodes one or both of the following amino acid sequences:
(vi) an amino acid sequence comprising Melanoma antigen C1 (MAGE-C1), an immunogenic variant thereof, or an immunogenic fragment of the MAGE-C1 or the immunogenic variant thereof; and
(vii) an amino acid sequence comprising New York esophageal squamous cell carcinoma-1 (NY-ESO-1), an immunogenic variant thereof, or an immunogenic fragment of the NY-ESO-1 or the immunogenic variant thereof.

In one embodiment, the at least one RNA further encodes:
(vi) an amino acid sequence comprising Melanoma antigen C1 (MAGE-C1), an immunogenic variant thereof, or an immunogenic fragment of the MAGE-C1 or the immunogenic variant thereof.

In one embodiment, the at least one RNA encodes:
(i) an amino acid sequence comprising claudin 6 (CLDN6), an immunogenic variant thereof, or an immunogenic fragment of the CLDN6 or the immunogenic variant thereof;
(ii) an amino acid sequence comprising Kita-kyushu lung cancer antigen 1 (KK-LC-1), an immunogenic variant thereof, or an immunogenic fragment of the KK-LC-1 or the immunogenic variant thereof;
(iii) an amino acid sequence comprising Melanoma antigen A3 (MAGE-A3), an immunogenic variant thereof, or an immunogenic fragment of the MAGE-A3 or the immunogenic variant thereof;
(iv) an amino acid sequence comprising Melanoma antigen 4 (MAGE-A4), an immunogenic variant thereof, or an immunogenic fragment of the MAGE-A4 or the immunogenic variant thereof;
(v) an amino acid sequence comprising Preferentially Expressed Antigen In Melanoma (PRAME), an immunogenic variant thereof, or an immunogenic fragment of the PRAME or the immunogenic variant thereof; and
(vi) an amino acid sequence comprising Melanoma antigen C1 (MAGE-C1), an immunogenic variant thereof, or an immunogenic fragment of the MAGE-C1 or the immunogenic variant thereof.

In one embodiment, each of the amino acid sequences under (i), (ii), (iii), (iv), (v), (vi), or (vii) is encoded by a separate RNA.

In one embodiment,
(i) the RNA encoding the amino acid sequence under (i) comprises the nucleotide sequence of SEQ ID NO: 3 or 4, or a nucleotide sequence having at least 99%, 98%, 97%, 96%, 95%, 90%, 85%, or 80% identity to the nucleotide sequence of SEQ ID NO: 3 or 4; and/or
(ii) the amino acid sequence under (i) comprises the amino acid sequence of SEQ ID NO: 1 or 2, or an amino acid sequence having at least 99%, 98%, 97%, 96%, 95%, 90%, 85%, or 80% identity to the amino acid sequence of SEQ ID NO: 1 or 2.

In one embodiment,
(i) the RNA encoding the amino acid sequence under (ii) comprises the nucleotide sequence of SEQ ID NO: 7 or 8, or a nucleotide sequence having at least 99%, 98%, 97%, 96%, 95%, 90%, 85%, or 80% identity to the nucleotide sequence of SEQ ID NO: 7 or 8; and/or
(ii) the amino acid sequence under (ii) comprises the amino acid sequence of SEQ ID NO: 5 or 6, or an amino acid sequence having at least 99%, 98%, 97%, 96%, 95%, 90%, 85%, or 80% identity to the amino acid sequence of SEQ ID NO: 5 or 6.

In one embodiment,
(i) the RNA encoding the amino acid sequence under (iii) comprises the nucleotide sequence of SEQ ID NO: 11 or 12, or a nucleotide sequence having at least 99%, 98%, 97%, 96%, 95%, 90%, 85%, or 80% identity to the nucleotide sequence of SEQ ID NO: 11 or 12; and/or
(ii) the amino acid sequence under (iii) comprises the amino acid sequence of SEQ ID NO: 9 or 10, or an amino acid sequence having at least 99%, 98%, 97%, 96%, 95%, 90%, 85%, or 80% identity to the amino acid sequence of SEQ ID NO: 9 or 10.

In one embodiment,
(i) the RNA encoding the amino acid sequence under (iv) comprises the nucleotide sequence of SEQ ID NO: 15 or 16, or a nucleotide sequence having at least 99%, 98%, 97%, 96%, 95%, 90%, 85%, or 80% identity to the nucleotide sequence of SEQ ID NO: 15 or 16; and/or
(ii) the amino acid sequence under (iv) comprises the amino acid sequence of SEQ ID NO: 13 or 14, or an amino acid sequence having at least 99%, 98%, 97%, 96%, 95%, 90%, 85%, or 80% identity to the amino acid sequence of SEQ ID NO: 13 or 14.

In one embodiment,
(i) the RNA encoding the amino acid sequence under (v) comprises the nucleotide sequence of SEQ ID NO: 19 or 20, or a nucleotide sequence having at least 99%, 98%, 97%, 96%, 95%, 90%, 85%, or 80% identity to the nucleotide sequence of SEQ ID NO: 19 or 20; and/or
(ii) the amino acid sequence under (v) comprises the amino acid sequence of SEQ ID NO: 17 or 18, or an amino acid sequence having at least 99%, 98%, 97%, 96%, 95%, 90%, 85%, or 80% identity to the amino acid sequence of SEQ ID NO: 17 or 18.

In one embodiment,
(i) the RNA encoding the amino acid sequence under (vi) comprises the nucleotide sequence of SEQ ID NO: 23 or 24, or a nucleotide sequence having at least 99%, 98%, 97%, 96%, 95%, 90%, 85%, or 80% identity to the nucleotide sequence of SEQ ID NO: 23 or 24; and/or
(ii) the amino acid sequence under (vi) comprises the amino acid sequence of SEQ ID NO: 21 or 22, or an amino acid sequence having at least 99%, 98%, 97%, 96%, 95%, 90%, 85%, or 80% identity to the amino acid sequence of SEQ ID NO: 21 or 22.

In one embodiment,
(i) the RNA encoding the amino acid sequence under (vii) comprises the nucleotide sequence of SEQ ID NO: 27 or 28, or a nucleotide sequence having at least 99%, 98%, 97%, 96%, 95%, 90%, 85%, or 80% identity to the nucleotide sequence of SEQ ID NO: 27 or 28; and/or
(ii) the amino acid sequence under (vii) comprises the amino acid sequence of SEQ ID NO: 25 or 26, or an amino acid sequence having at least 99%, 98%, 97%, 96%, 95%, 90%, 85%, or 80% identity to the amino acid sequence of SEQ ID NO: 25 or 26.

In one embodiment, at least one amino acid sequence under (i), (ii), (iii), (iv), (v), (vi), or (vii) comprises an amino acid sequence which breaks immunological tolerance and/or at least one RNA is co-administered with RNA encoding an amino acid sequence which breaks immunological tolerance. In one embodiment, each amino acid sequence under (i), (ii), (iii), (iv), (v), (vi), or (vii) comprises an amino acid sequence which breaks immunological tolerance and/or each RNA is co-administered with RNA encoding an amino acid sequence which breaks immunological tolerance. In one embodiment, the amino acid sequence which breaks immunological tolerance comprises helper epitopes, preferably tetanus toxoid-derived helper epitopes. In one embodiment,
(i) the RNA encoding the amino acid sequence which breaks immunological tolerance comprises the nucleotide sequence of SEQ ID NO: 34, or a nucleotide sequence having at least 99%, 98%, 97%, 96%, 95%, 90%, 85%, or 80% identity to the nucleotide sequence of SEQ ID NO: 34; and/or
(ii) the amino acid sequence which breaks immunological tolerance comprises the amino acid sequence of SEQ ID NO: 33, or an amino acid sequence having at least 99%, 98%, 97%, 96%, 95%, 90%, 85%, or 80% identity to the amino acid sequence of SEQ ID NO: 33.

In one embodiment, at least one of the amino acid sequences under (i), (ii), (iii), (iv), (v), (vi), or (vii) is encoded by a coding sequence which is codon-optimized and/or the G/C content of which is increased compared to wild type coding sequence, wherein the codon-optimization and/or the increase in the G/C content preferably does not change the sequence of the encoded amino acid sequence. In one embodiment, each of the amino acid sequences under (i), (ii), (iii), (iv), (v), (vi), or (vii) is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In one embodiment, at least one RNA is a modified RNA, in particular a stabilized mRNA. In one embodiment, at least one RNA comprises a modified nucleoside in place of at least one uridine. In one embodiment, at least one RNA comprises a modified nucleoside in place of each uridine. In one embodiment, each RNA comprises a modified nucleoside in place of at least one uridine. In one embodiment, each RNA comprises a modified nucleoside in place of each uridine. In one embodiment, the modified nucleoside is independently selected from pseudouridine (ψ), N1-methyl-pseudouridine (m1ψ), and 5-methyl-uridine (m5U).

In one embodiment, at least one RNA comprises the 5′ cap m₂^{7,2′-O}Gppsp(5′)G. In one embodiment, each RNA comprises the 5′ cap m₂^{7,2′-O}Gppₛp(5′)G.

In one embodiment, at least one RNA comprises a 5′ UTR comprising the nucleotide sequence of SEQ ID NO: 35, or a nucleotide sequence having at least 99%, 98%, 97%, 96%, 95%, 90%, 85%, or 80% identity to the nucleotide sequence of SEQ ID NO: 35. In one embodiment, each RNA comprises a 5′ UTR comprising the nucleotide sequence of SEQ ID NO: 35, or a nucleotide sequence having at least 99%, 98%, 97%, 96%, 95%, 90%, 85%, or 80% identity to the nucleotide sequence of SEQ ID NO: 35.

In one embodiment, at least one amino acid sequence under (i), (ii), (iii), (iv), (v), (vi), or (vii) comprises an amino acid sequence enhancing antigen processing and/or presentation. In one embodiment, each amino acid sequence under (iii), (iv), (v), (vi), or (vii) comprises an amino acid sequence enhancing antigen processing and/or presentation. In one embodiment, each amino acid sequence under (i), (ii), (iii), (iv), (v), (vi), or (vii) comprises an amino acid sequence enhancing antigen processing and/or presentation. In one embodiment, the amino acid sequence enhancing antigen processing and/or presentation comprises an amino acid sequence corresponding to the transmembrane and cytoplasmic domain of a MHC molecule, preferably a MHC class I molecule. In one embodiment,
(i) the RNA encoding the amino acid sequence enhancing antigen processing and/or presentation comprises the nucleotide sequence of SEQ ID NO: 32, or a nucleotide sequence having at least 99%, 98%, 97%, 96%, 95%, 90%, 85%, or 80% identity to the nucleotide sequence of SEQ ID NO: 32; and/or
(ii) the amino acid sequence enhancing antigen processing and/or presentation comprises the amino acid sequence of SEQ ID NO: 31, or an amino acid sequence having at least 99%, 98%, 97%, 96%, 95%, 90%, 85%, or 80% identity to the amino acid sequence of SEQ ID NO: 31.

In one embodiment, the amino acid sequence enhancing antigen processing and/or presentation further comprises an amino acid sequence coding for a secretory signal peptide.

In one embodiment,
(i) the RNA encoding the secretory signal peptide comprises the nucleotide sequence of SEQ ID NO: 30, or a nucleotide sequence having at least 99%, 98%, 97%, 96%, 95%, 90%, 85%, or 80% identity to the nucleotide sequence of SEQ ID NO: 30; and/or
(ii) the secretory signal peptide comprises the amino acid sequence of SEQ ID NO: 29, or an amino acid sequence having at least 99%, 98%, 97%, 96%, 95%, 90%, 85%, or 80% identity to the amino acid sequence of SEQ ID NO: 29.

In one embodiment, at least one RNA comprises a 3′ UTR comprising the nucleotide sequence of SEQ ID NO: 36, or a nucleotide sequence having at least 99%, 98%, 97%, 96%, 95%, 90%, 85%, or 80% identity to the nucleotide sequence of SEQ ID NO: 36. In one embodiment, each RNA comprises a 3′ UTR comprising the nucleotide sequence of SEQ ID NO: 36, or a nucleotide sequence having at least 99%, 98%, 97%, 96%, 95%, 90%, 85%, or 80% identity to the nucleotide sequence of SEQ ID NO: 36.

In one embodiment, at least one RNA comprises a poly-A sequence. In one embodiment, each RNA comprises a poly-A sequence. In one embodiment, the poly-A sequence comprises at least 100 nucleotides. In one embodiment, the poly-A sequence comprises or consists of the nucleotide sequence of SEQ ID NO: 37.

In one embodiment, the RNA is formulated as a liquid, formulated as a solid, or a combination thereof. In one embodiment, the RNA is formulated for injection. In one embodiment, the RNA is formulated for intravenous administration.

In one embodiment, the RNA is formulated or is to be formulated as lipoplex particles. In one embodiment, the RNA lipoplex particles are obtainable by mixing the RNA with liposomes. In one embodiment, at least one RNA encoding an amino acid sequence under (i), (ii), (iii), (iv), (v), (vi), and/or (vii) is co-formulated or is to be co-formulated as lipoplex particles with RNA encoding an amino acid sequence which breaks immunological tolerance. In one embodiment, each RNA encoding an amino acid sequence under (i), (ii), (iii), (iv), (v), (vi), and/or (vii) is co-formulated or is to be co-formulated as lipoplex particles with RNA encoding an amino acid sequence which breaks immunological tolerance. In one embodiment, the RNA encoding an amino acid sequence under (i), (ii), (iii), (iv), (v), (vi), and/or (vii) is co-formulated or is to be co-formulated as lipoplex particles with the RNA encoding an amino acid sequence which breaks immunological tolerance at a ratio of about 4:1 to about 16:1, about 6:1 to about 14:1, about 8:1 to about 12:1, or about 10:1.

In one embodiment, the composition or medical preparation comprises:
(i) RNA encoding an amino acid sequence comprising the amino acid sequence of SEQ ID NO: 2;
(ii) RNA encoding an amino acid sequence comprising the amino acid sequence of SEQ ID NO: 6;
(iii) RNA encoding an amino acid sequence comprising the amino acid sequence of SEQ ID NO: 10;
(iv) RNA encoding an amino acid sequence comprising the amino acid sequence of SEQ ID NO: 14;
(v) RNA encoding an amino acid sequence comprising the amino acid sequence of SEQ ID NO: 18; and
(vi) RNA encoding an amino acid sequence comprising the amino acid sequence of SEQ ID NO: 22.

In one embodiment, the composition or medical preparation comprises:
(i) RNA comprising the nucleotide sequence of SEQ ID NO: 4;
(ii) RNA comprising the nucleotide sequence of SEQ ID NO: 8;
(iii) RNA comprising the nucleotide sequence of SEQ ID NO: 12;
(iv) RNA comprising the nucleotide sequence of SEQ ID NO: 16;
(v) RNA comprising the nucleotide sequence of SEQ ID NO: 20; and
(vi) RNA comprising the nucleotide sequence of SEQ ID NO: 24.

In one embodiment, the composition or medical preparation is a pharmaceutical composition.

In one embodiment, the pharmaceutical composition further comprises one or more pharmaceutically acceptable carriers, diluents and/or excipients.

In one embodiment, the medical preparation is a kit. In one embodiment, the RNAs are in separate vials.

In one embodiment, the composition or medical preparation further comprises instructions for use of the composition or medical preparation for treating or preventing lung cancer.

In one embodiment, the composition or medical preparation is for pharmaceutical use. In one embodiment, the pharmaceutical use comprises a therapeutic or prophylactic treatment of a disease or disorder. In one embodiment, the therapeutic or prophylactic treatment of a disease or disorder comprises treating or preventing lung cancer. In one embodiment, the composition or medical preparation is for administration to a human.

In a further aspect, the invention relates to a method of treating lung cancer in a subject comprising administering at least one RNA to the subject, wherein the at least one RNA encodes the following amino acid sequences:
(i) an amino acid sequence comprising claudin 6 (CLDN6), an immunogenic variant thereof, or an immunogenic fragment of the CLDN6 or the immunogenic variant thereof;
(ii) an amino acid sequence comprising Kita-kyushu lung cancer antigen 1 (KK-LC-1), an immunogenic variant thereof, or an immunogenic fragment of the KK-LC-1 or the immunogenic variant thereof;
(iii) an amino acid sequence comprising Melanoma antigen A3 (MAGE-A3), an immunogenic variant thereof, or an immunogenic fragment of the MAGE-A3 or the immunogenic variant thereof;
(iv) an amino acid sequence comprising Melanoma antigen 4 (MAGE-A4), an immunogenic variant thereof, or an immunogenic fragment of the MAGE-A4 or the immunogenic variant thereof; and
(v) an amino acid sequence comprising Preferentially Expressed Antigen In Melanoma (PRAME), an immunogenic variant thereof, or an immunogenic fragment of the PRAME or the immunogenic variant thereof.

In one embodiment, the at least one RNA further encodes one or both of the following amino acid sequences:
(vi) an amino acid sequence comprising Melanoma antigen C1 (MAGE-C1), an immunogenic variant thereof, or an immunogenic fragment of the MAGE-C1 or the immunogenic variant thereof; and
(vii) an amino acid sequence comprising New York esophageal squamous cell carcinoma-1 (NY-ESO-1), an immunogenic variant thereof, or an immunogenic fragment of the NY-ESO-1 or the immunogenic variant thereof.

In one embodiment, the at least one RNA further encodes:
(vi) an amino acid sequence comprising Melanoma antigen C1 (MAGE-C1), an immunogenic variant thereof, or an immunogenic fragment of the MAGE-C1 or the immunogenic variant thereof.

In one embodiment, the at least one RNA encodes:
(i) an amino acid sequence comprising claudin 6 (CLDN6), an immunogenic variant thereof, or an immunogenic fragment of the CLDN6 or the immunogenic variant thereof;
(ii) an amino acid sequence comprising Kita-kyushu lung cancer antigen 1 (KK-LC-1), an immunogenic variant thereof, or an immunogenic fragment of the KK-LC-1 or the immunogenic variant thereof;
(iii) an amino acid sequence comprising Melanoma antigen A3 (MAGE-A3), an immunogenic variant thereof, or an immunogenic fragment of the MAGE-A3 or the immunogenic variant thereof;
(iv) an amino acid sequence comprising Melanoma antigen 4 (MAGE-A4), an immunogenic variant thereof, or an immunogenic fragment of the MAGE-A4 or the immunogenic variant thereof;
(v) an amino acid sequence comprising Preferentially Expressed Antigen In Melanoma (PRAME), an immunogenic variant thereof, or an immunogenic fragment of the PRAME or the immunogenic variant thereof; and
(vi) an amino acid sequence comprising Melanoma antigen C1 (MAGE-C1), an immunogenic variant thereof, or an immunogenic fragment of the MAGE-C1 or the immunogenic variant thereof.

In one embodiment, each of the amino acid sequences under (i), (ii), (iii), (iv), (v), (vi), or (vii) is encoded by a separate RNA.

In one embodiment,
(i) the RNA encoding the amino acid sequence under (i) comprises the nucleotide sequence of SEQ ID NO: 3 or 4, or a nucleotide sequence having at least 99%, 98%, 97%, 96%, 95%, 90%, 85%, or 80% identity to the nucleotide sequence of SEQ ID NO: 3 or 4; and/or
(ii) the amino acid sequence under (i) comprises the amino acid sequence of SEQ ID NO: 1 or 2, or an amino acid sequence having at least 99%, 98%, 97%, 96%, 95%, 90%, 85%, or 80% identity to the amino acid sequence of SEQ ID NO: 1 or 2.

In one embodiment,
(i) the RNA encoding the amino acid sequence under (ii) comprises the nucleotide sequence of SEQ ID NO: 7 or 8, or a nucleotide sequence having at least 99%, 98%, 97%, 96%, 95%, 90%, 85%, or 80% identity to the nucleotide sequence of SEQ ID NO: 7 or 8; and/or
(ii) the amino acid sequence under (ii) comprises the amino acid sequence of SEQ ID NO: 5 or 6, or an amino acid sequence having at least 99%, 98%, 97%, 96%, 95%, 90%, 85%, or 80% identity to the amino acid sequence of SEQ ID NO: 5 or 6.

In one embodiment,
(i) the RNA encoding the amino acid sequence under (iii) comprises the nucleotide sequence of SEQ ID NO: 11 or 12, or a nucleotide sequence having at least 99%, 98%, 97%, 96%, 95%, 90%, 85%, or 80% identity to the nucleotide sequence of SEQ ID NO: 11 or 12; and/or
(ii) the amino acid sequence under (iii) comprises the amino acid sequence of SEQ ID NO: 9 or 10, or an amino acid sequence having at least 99%, 98%, 97%, 96%, 95%, 90%, 85%, or 80% identity to the amino acid sequence of SEQ ID NO: 9 or 10.

In one embodiment,
(i) the RNA encoding the amino acid sequence under (iv) comprises the nucleotide sequence of SEQ ID NO: 15 or 16, or a nucleotide sequence having at least 99%, 98%, 97%, 96%, 95%, 90%, 85%, or 80% identity to the nucleotide sequence of SEQ ID NO: 15 or 16; and/or
(ii) the amino acid sequence under (iv) comprises the amino acid sequence of SEQ ID NO: 13 or 14, or an amino acid sequence having at least 99%, 98%, 97%, 96%, 95%, 90%, 85%, or 80% identity to the amino acid sequence of SEQ ID NO: 13 or 14.

In one embodiment,
(i) the RNA encoding the amino acid sequence under (v) comprises the nucleotide sequence of SEQ ID NO: 19 or 20, or a nucleotide sequence having at least 99%, 98%, 97%, 96%, 95%, 90%, 85%, or 80% identity to the nucleotide sequence of SEQ ID NO: 19 or 20; and/or
(ii) the amino acid sequence under (v) comprises the amino acid sequence of SEQ ID NO: 17 or 18, or an amino acid sequence having at least 99%, 98%, 97%, 96%, 95%, 90%, 85%, or 80% identity to the amino acid sequence of SEQ ID NO: 17 or 18.

In one embodiment,
(i) the RNA encoding the amino acid sequence under (vi) comprises the nucleotide sequence of SEQ ID NO: 23 or 24, or a nucleotide sequence having at least 99%, 98%, 97%, 96%, 95%, 90%, 85%, or 80% identity to the nucleotide sequence of SEQ ID NO: 23 or 24; and/or
(ii) the amino acid sequence under (vi) comprises the amino acid sequence of SEQ ID NO: 21 or 22, or an amino acid sequence having at least 99%, 98%, 97%, 96%, 95%, 90%, 85%, or 80% identity to the amino acid sequence of SEQ ID NO: 21 or 22.

In one embodiment,
(i) the RNA encoding the amino acid sequence under (vii) comprises the nucleotide sequence of SEQ ID NO: 27 or 28, or a nucleotide sequence having at least 99%, 98%, 97%, 96%, 95%, 90%, 85%, or 80% identity to the nucleotide sequence of SEQ ID NO: 27 or 28; and/or
(ii) the amino acid sequence under (vii) comprises the amino acid sequence of SEQ ID NO: 25 or 26, or an amino acid sequence having at least 99%, 98%, 97%, 96%, 95%, 90%, 85%, or 80% identity to the amino acid sequence of SEQ ID NO: 25 or 26.

In one embodiment, at least one amino acid sequence under (i), (ii), (iii), (iv), (v), (vi), or (vii) comprises an amino acid sequence which breaks immunological tolerance and/or at least one RNA is co-administered with RNA encoding an amino acid sequence which breaks immunological tolerance. In one embodiment, each amino acid sequence under (i), (ii), (iii), (iv), (v), (vi), or (vii) comprises an amino acid sequence which breaks immunological tolerance and/or each RNA is co-administered with RNA encoding an amino acid sequence which breaks immunological tolerance. In one embodiment, the amino acid sequence which breaks immunological tolerance comprises helper epitopes, preferably tetanus toxoid-derived helper epitopes. In one embodiment,
(i) the RNA encoding the amino acid sequence which breaks immunological tolerance comprises the nucleotide sequence of SEQ ID NO: 34, or a nucleotide sequence having at least 99%, 98%, 97%, 96%, 95%, 90%, 85%, or 80% identity to the nucleotide sequence of SEQ ID NO: 34; and/or
(ii) the amino acid sequence which breaks immunological tolerance comprises the amino acid sequence of SEQ ID NO: 33, or an amino acid sequence having at least 99%, 98%, 97%, 96%, 95%, 90%, 85%, or 80% identity to the amino acid sequence of SEQ ID NO: 33.

In one embodiment, at least one of the amino acid sequences under (i), (ii), (iii), (iv), (v), (vi), or (vii) is encoded by a coding sequence which is codon-optimized and/or the G/C content of which is increased compared to wild type coding sequence, wherein the codon-optimization and/or the increase in the G/C content preferably does not change the sequence of the encoded amino acid sequence. In one embodiment, each of the amino acid sequences under (i), (ii), (iii), (iv), (v), (vi), or (vii) is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

In one embodiment, at least one RNA is a modified RNA, in particular a stabilized mRNA. In one embodiment, at least one RNA comprises a modified nucleoside in place of at least one uridine. In one embodiment, at least one RNA comprises a modified nucleoside in place of each uridine. In one embodiment, each RNA comprises a modified nucleoside in place of at least one uridine. In one embodiment, each RNA comprises a modified nucleoside in place of each uridine. In one embodiment, the modified nucleoside is independently selected from pseudouridine (ψ), N1-methyl-pseudouridine (m1ψ), and 5-methyl-uridine (m5U).

In one embodiment, at least one RNA comprises the 5′ cap m₂^{7,2′-O}Gppsp(5′)G. In one embodiment, each RNA comprises the 5′ cap m₂^{7,2′-O}Gppsp(5′)G.

In one embodiment, at least one RNA comprises a 5′ UTR comprising the nucleotide sequence of SEQ ID NO: 35, or a nucleotide sequence having at least 99%, 98%, 97%, 96%, 95%, 90%, 85%, or 80% identity to the nucleotide sequence of SEQ ID NO: 35. In one embodiment, each RNA comprises a 5′ UTR comprising the nucleotide sequence of SEQ ID NO: 35, or a nucleotide sequence having at least 99%, 98%, 97%, 96%, 95%, 90%, 85%, or 80% identity to the nucleotide sequence of SEQ ID NO: 35.

In one embodiment, at least one amino acid sequence under (i), (ii), (iii), (iv), (v), (vi), or (vii) comprises an amino acid sequence enhancing antigen processing and/or presentation. In one embodiment, each amino acid sequence under (iii), (iv), (v), (vi), or (vii) comprises an amino acid sequence enhancing antigen processing and/or presentation. In one embodiment, each amino acid sequence under (i), (ii), (iii), (iv), (v), (vi), or (vii) comprises an amino acid sequence enhancing antigen processing and/or presentation. In one embodiment, the amino acid sequence enhancing antigen processing and/or presentation comprises an amino acid sequence corresponding to the transmembrane and cytoplasmic domain of a MHC molecule, preferably a MHC class I molecule. In one embodiment,
(i) the RNA encoding the amino acid sequence enhancing antigen processing and/or presentation comprises the nucleotide sequence of SEQ ID NO: 32, or a nucleotide sequence having at least 99%, 98%, 97%, 96%, 95%, 90%, 85%, or 80% identity to the nucleotide sequence of SEQ ID NO: 32; and/or
(ii) the amino acid sequence enhancing antigen processing and/or presentation comprises the amino acid sequence of SEQ ID NO: 31, or an amino acid sequence having at least 99%, 98%, 97%, 96%, 95%, 90%, 85%, or 80% identity to the amino acid sequence of SEQ ID NO: 31.

In one embodiment, the amino acid sequence enhancing antigen processing and/or presentation further comprises an amino acid sequence coding for a secretory signal peptide.

In one embodiment,
(i) the RNA encoding the secretory signal peptide comprises the nucleotide sequence of SEQ ID NO: 30, or a nucleotide sequence having at least 99%, 98%, 97%, 96%, 95%, 90%, 85%, or 80% identity to the nucleotide sequence of SEQ ID NO: 30; and/or
(ii) the secretory signal peptide comprises the amino acid sequence of SEQ ID NO: 29, or an amino acid sequence having at least 99%, 98%, 97%, 96%, 95%, 90%, 85%, or 80% identity to the amino acid sequence of SEQ ID NO: 29.

In one embodiment, at least one RNA comprises a 3′ UTR comprising the nucleotide sequence of SEQ ID NO: 36, or a nucleotide sequence having at least 99%, 98%, 97%, 96%, 95%, 90%, 85%, or 80% identity to the nucleotide sequence of SEQ ID NO: 36. In one embodiment, each RNA comprises a 3′ UTR comprising the nucleotide sequence of SEQ ID NO: 36, or a nucleotide sequence having at least 99%, 98%, 97%, 96%, 95%, 90%, 85%, or 80% identity to the nucleotide sequence of SEQ ID NO: 36.

In one embodiment, at least one RNA comprises a poly-A sequence. In one embodiment, each RNA comprises a poly-A sequence. In one embodiment, the poly-A sequence comprises at least 100 nucleotides. In one embodiment, the poly-A sequence comprises or consists of the nucleotide sequence of SEQ ID NO: 37.

In one embodiment, the RNA is administered by injection. In one embodiment, the RNA is administered by intravenous administration.

In one embodiment, the RNA is formulated as lipoplex particles. In one embodiment, the RNA lipoplex particles are obtainable by mixing the RNA with liposomes. In one embodiment, at least one RNA encoding an amino acid sequence under (i), (ii), (iii), (iv), (v), (vi), and/or (vii) is co-formulated as lipoplex particles with RNA encoding an amino acid sequence which breaks immunological tolerance. In one embodiment, each RNA encoding an amino acid sequence under (i), (ii), (iii), (iv), (v), (vi), and/or (vii) is co-formulated as lipoplex particles with RNA encoding an amino acid sequence which breaks immunological tolerance. In one embodiment, the RNA encoding an amino acid sequence under (i), (ii), (iii), (iv), (v), (vi), and/or (vii) is co-formulated as lipoplex particles with the RNA encoding an amino acid sequence which breaks immunological tolerance at a ratio of about 4:1 to about 16:1, about 6:1 to about 14:1, about 8:1 to about 12:1, or about 10:1.

In one embodiment, the method comprises administering:
(i) RNA encoding an amino acid sequence comprising the amino acid sequence of SEQ ID NO: 2;
(ii) RNA encoding an amino acid sequence comprising the amino acid sequence of SEQ ID NO: 6;
(iii) RNA encoding an amino acid sequence comprising the amino acid sequence of SEQ ID NO: 10;
(iv) RNA encoding an amino acid sequence comprising the amino acid sequence of SEQID NO: 14;
(v) RNA encoding an amino acid sequence comprising the amino acid sequence of SEQ ID NO: 18; and
(vi) RNA encoding an amino acid sequence comprising the amino acid sequence of SEQ ID NO: 22.

In one embodiment, the method comprises administering:
(i) RNA comprising the nucleotide sequence of SEQ ID NO: 4;
(ii) RNA comprising the nucleotide sequence of SEQ ID NO: 8;
(iii) RNA comprising the nucleotide sequence of SEQ ID NO: 12;
(iv) RNA comprising the nucleotide sequence of SEQ ID NO: 16;
(v) RNA comprising the nucleotide sequence of SEQ ID NO: 20; and
(vi) RNA comprising the nucleotide sequence of SEQ ID NO: 24.

In one embodiment, the subject is a human.

In one aspect, provided herein is RNA described herein, e.g.,
(i) RNA encoding an amino acid sequence comprising claudin 6 (CLDN6), an immunogenic variant thereof, or an immunogenic fragment of the CLDN6 or the immunogenic variant thereof;
(ii) RNA encoding an amino acid sequence comprising Kita-kyushu lung cancer antigen 1 (KK-LC-1), an immunogenic variant thereof, or an immunogenic fragment of the KK-LC-1 or the immunogenic variant thereof;
(iii) RNA encoding an amino acid sequence comprising Melanoma antigen A3 (MAGE-A3), an immunogenic variant thereof, or an immunogenic fragment of the MAGE-A3 or the immunogenic variant thereof;
(iv) RNA encoding an amino acid sequence comprising Melanoma antigen 4 (MAGE-A4), an immunogenic variant thereof, or an immunogenic fragment of the MAGE-A4 or the immunogenic variant thereof; and
(v) RNA encoding an amino acid sequence comprising Preferentially Expressed Antigen In Melanoma (PRAME), an immunogenic variant thereof, or an immunogenic fragment of the PRAME or the immunogenic variant thereof;
   and optionally one or more of:
(vi) RNA encoding an amino acid sequence comprising Melanoma antigen C1 (MAGE-C1), an immunogenic variant thereof, or an immunogenic fragment of the MAGE-C1 or the immunogenic variant thereof; and/or
(vii) RNA encoding an amino acid sequence comprising New York esophageal squamous cell carcinoma-1 (NY-ESO-1), an immunogenic variant thereof, or an immunogenic fragment of the NY-ESO-1 or the immunogenic variant thereof, for use in a method described herein.

Embodiments of the RNA for the use are as described herein, e.g., in respect of the composition or medical preparation or the method of the invention.

### BRIEF DESCRIPTION OF THE DRAWINGS

FIG. 1: RNA expression intensities of target genes in 881 NSCLC tumors and 37 normal tissue sites.

Expression values were calculated from RNA sequencing data of lung adenocarcinoma (LUAD), lung squamous cell carcinoma (LUSC) and normal tissue sites in reads per kilobase million (rpkm).

FIG. 2: Tumor percentage expressing the targets and cumulative coverage across 881 NSCLC tumors.

RNA sequencing expression data and cutoff for positive tumors were included to compare individual target expressing tumor percentage and cumulative coverage achieved by target combination. The top numbers represented present, absent and target-expressing values. The targets were ranked from left to right by the highest added value to increase the cumulative coverage.

FIG. 3: Tumor fractions expressing at least two, three or more targets dependent on four different target sets across 881 NSCLC tumors.

The 5 core target set includes KK-LC-1, MAGEA3, PRAME, MAGEA4, and CLDN6 as a minimal set of targets that covers about 60% of tumors with at least two out of the 5 targets. The two 6 target sets includes either MAGEC1 or NY-ESO-1. The 7 target set includes all given targets.

FIG. 4: RNA expression of targets in 164 NSCLC and other lung tumors, and 43 normal tissue sites.

Expression values were calculated from quantitative real-time PCR data of lung adenocarcinoma (LUAD), lung squamous cell carcinoma (LUSC), other lung tumors, and normal tissue sites. Normalized expression values are given in arbitrary units (a.u.).

FIG. 5: Tumor percentage expressing the targets and cumulative coverage across 164 NSCLC and other lung tumors.

qRT-PCR expression data and target-specific cutoffs for positive tumors were included to compare individual target expressing tumor percentage and cumulative coverage achieved by target combination. The top numbers represented present, absent and target-expressing values. The targets were ranked from left to right by the highest added value to increase the cumulative coverage.

FIG. 6: Tumor fractions expressing at least two, three or more targets dependent on four different target sets across 164 NSCLC and other lung tumors.

The 5 core target set includes KK-LC-1, MAGEA3, PRAME, MAGEA4, and CLDN6 as a minimal set of targets that covers about 60% of tumors with at least two out of the 5 targets. The two 6 target sets includes either MAGEC1 or NY-ESO-1. The 7 target set includes all given targets.

FIG. 7: Induction of antigen-specific T cells in the spleen by MAGEA3-, KK-LC-1-, CLDN6-, NY-ESO-1-, MAGEA4-, PRAME- and MAGEC1-coding RNA.

IFN-γ ELISPOT analysis of T-cell effectors from the spleen of mice immunized with lipoplex-formulated RNA coding for MAGEA3, KK-LC-1, CLDN6, NY-ESO-1, MAGEA4, PRAME and MAGEC1. Splenocytes obtained five days after the final immunization were re-stimulated with either a peptide pool spanning the respective human protein or with an irrelevant control peptide. In case of MAGEC1 RNA, splenocytes stimulation was performed using electroporated cultivated mouse BMDCs, electroporated with either antigen-coding RNA for MAGEC1 or irrelevant RNA as negative control. Dots represent individual animals; horizontal bars indicate the mean±SD of the three animals.

FIG. 8: Vaccine-induced CD8+ and CD4⁺ T-cell responses against KKLC1, CLDN6 (A) and PRAME (B). Ex vivo T-cell responses of patient WO5YAH (A) and AW8VMT (B) pre (V1) and post (FU) 8 vaccinations were measured after pulsing PBMCs with individual TAA PepMix.

Negative control, PBMCs/cells only: PBMCs incubated with medium; positive control, PBMCs incubated with anti-CD3 antibody.

FIG. 9: Overview of the process for analyzing gene expression by RT-qPCR.

FIG. 10: De novo antigen-specific CD8*T cell induction by BNT116 in human HLA-transgenic, A2/DR1 mice

C57BL/6 A2/DR1 mice were vaccinated three times IV with 2 μg MAGE-A3 RNA-LPX (RBL003.3 [research-grade], n=5) (A), or PRAME, CLDN6, KK-LC-1, MAGE-A4, or MAGE-C1 RNA-LPX (n=3 per group) (B) (RBL012.2, RBL005.3, RBL007.2, RBLO27.2, or RBLO35.2 [CTM], respectively), on Days 1, 8 and 15. The induction of antigen-specific T cells was analyzed on Day 20 by IFN-γ production of splenocytes after ex vivo restimulation with BNT116 peptide mixes, or P2P16P17 peptide mix, spanning the helper epitopes P2P16, by ELISpot. Controls were restimulated with irrelevant human cytomegalovirus (hCMV) pp65₄₉₅₋₅₀₄ peptide. Individual data points represent means of triplicates per mouse. Horizontal lines and error bars indicate the mean of each group±SEM. Restimulation of splenocytes from one mouse in the PRAME RNA-LPX immunized group with PRAME PepMix resulted in IFN-γ spot numbers too numerous to count. A spot number of 1,700 was assumed in order to perform statistical analysis (B). Statistical significance between groups restimulated with cognate or irrelevant peptide mixes was determined by one-way repeated measures ANOVA and Dunnett's multiple comparisons test. Note: Spot count sensitivity differed between the data sets in (A) and (B), and absolute values cannot be compared. *p 0.05, **p<0.01, ****p<0.0001.

ANOVA=analysis of variance; CTM=clinical trial material; ELISpot=enzyme-linked immune absorbent spot; hCMV=human cytomegalovirus; IFN=interferon; IV=intravenous; RNA-LPX=ribonucleic acid lipoplex.

Source: Study No. R-21-0164 (A), R-21-0358 (B).

FIG. 11: De novo induction of antigen-specific T cells in human HLA-transgenic A2/DR1 mice by BNT116 administered within a single injection.

C57BL/6 A2/DR1 mice (n=6 per group) were vaccinated three times IV with a mixture of all six BNT116 RNAs (PRAME [RBL012.2], CLDN6 [RBL005.3], KK-LC-1 [RBL007.2], MAGE-3 [RBL003.3], MAGE-A4 [RBLO27.2] and MAGE-C1 [RBLO35.2]), either formulated first and then mixed (process 1), or mixed first and then formulated (process 2), on Days 1, 8 and 15. Mice receiving BNT116 according to process 1 were dosed with 10.8 μg per mouse, mice receiving BNT116 according to process 2 were dosed with 9.2 μg per mouse. The induction of antigen-specific T cells was analyzed on Day 20 by IFN-γ production of splenocytes after ex vivo restimulation with BNT116 peptide mixes, or P2P16P17 peptide mix, spanning the helper epitopes P2P16, by ELISpot. Control wells were restimulated with irrelevant human cytomegalovirus (hCMV) pp65495-504 peptide. Individual data points represent means of triplicates per mouse. Horizontal lines and error bars indicate the mean of each group±SEM. Outliers were removed according to Grubbs' outlier test (alpha=0.05; outlier removed in PRAME, process 2; KK-LC-1, process 1 and 2; MAGE-A3, process 2; MAGE-A4, process 2; Control, process 1). Statistical significance was determined by unpaired, two-tailed t test. **p≤0.01. Only significant differences are marked.

### Description of the Sequences

The following table provides a listing of certain sequences referenced herein.

DESCRIPTION OF THE SEQUENCES

| | | |
|---|---|---|
| SEQ | | |
| ID | | |
| NO: | Description | SEQUENCE |

CLDN6

| | | |
|---|---|---|
| 1 | CLDN6 | MASAGMQILGVVLTLLGWVNGLVSCALPMWKVTAFIGNSIVVAQVV |
| | (amino acid) | WEGLWMSCVVQSTGQMQCKVYDSLLALPQDLQAARALCVIALLVAL |
| | | FGLLVYLAGAKCTTCVEEKDSKARLVLTSGIVFVISGVLTLIPVCW |
| | | TAHAIIRDFYNPLVAEAQKRELGASLYLGWAASGLLLLGGGLLCCT |
| | | CPSGGSQGPSHYMARYSTSAPAISRGPSEYPTKNYV |
| 2 | CLDN6 fusion | MASAGMQILGVVLTLLGWVNGLVSCALPMWKVTAFIGNSIVVAQVV |
| | (amino acid) | WEGLWMSCVVQSTGQMQCKVYDSLLALPQDLQAARALCVIALLVAL |
| | | FGLLVYLAGAKCTTCVEEKDSKARLVLTSGIVFVISGVLTLIPVCW |
| | | TAHAIIRDFYNPLVAEAQKRELGASLYLGWAASGLLLLGGGLLCCT |
| | | CPSGGSQGPSHYMARYSTSAPAISRGPSEYPTKNYVGGSGGGGSGG |
| | | KKQYIKANSKFIGITELKKLGGGKRGGGKKMTNSVDDALINSTKIY |
| | | SYFPSVISKVNQGAQGKKL |
| 3 | CLDN6 (CDS) | AUGGCCUCUGCCGGAAUGCAGAUCCUGGGCGUGGUGCUGACCCUGC |
| | | UGGGCUGGGUGAAUGGCCUGGUGAGCUGUGCCCUGCCCAUGUGGAA |
| | | GGUGACAGCCUUCAUUGGCAACAGCAUUGUGGUGGCCCAGGUGGUG |
| | | UGGGAGGGCCUGUGGAUGAGCUGUGUGGUGCAGAGCACAGGCCAGA |
| | | UGCAGUGCAAGGUGUAUGACAGCCUGCUGGCCCUGCCUCAGGACCU |
| | | CCAGGCCGCCAGAGCCCUGUGUGUGAUUGCCCUGCUGGUGGCCCUG |
| | | UUUGGCCUGCUGGUGUACCUGGCUGGAGCCAAGUGCACCACCUGUG |
| | | UGGAGGAGAAGGACAGCAAGGCCAGACUGGUGCUGACCUCUGGCAU |
| | | UGUGUUUGUGAUCUCUGGCGUGCUGACCCUGAUCCCUGUGUGCUGG |
| | | ACAGCCCAUGCCAUCAUCAGAGACUUCUACAACCCUCUGGUGGCCG |
| | | AGGCCCAGAAAAGAGAGCUGGGAGCCAGCCUGUACCUGGGCUGGGC |
| | | CGCCUCUGGCCUUCUUCUGCUGGGAGGAGGACUGCUGUGCUGCACC |
| | | UGCCCCUCUGGCGGCAGCCAGGGCCCCAGCCACUACAUGGCCAGAU |
| | | ACAGCACCUCUGCCCCUGCCAUCAGCAGAGGCCCUUCUGAGUACCC |
| | | CACCAAGAACUAUGUG |
| 4 | CLDN6 (RNA) | GGGCGAACUAGUAUUCUUCUGGUCCCCACAGACUCAGAGAGAACCC |
| | | GCCACCAUGGCCUCUGCCGGAAUGCAGAUCCUGGGCGUGGUGCUGA |
| | | CCCUGCUGGGCUGGGUGAAUGGCCUGGUGAGCUGUGCCCUGCCCAU |
| | | GUGGAAGGUGACAGCCUUCAUUGGCAACAGCAUUGUGGUGGCCCAG |
| | | GUGGUGUGGGAGGGCCUGUGGAUGAGCUGUGUGGUGCAGAGCACAG |
| | | GCCAGAUGCAGUGCAAGGUGUAUGACAGCCUGCUGGCCCUGCCUCA |
| | | GGACCUCCAGGCCGCCAGAGCCCUGUGUGUGAUUGCCCUGCUGGUG |
| | | GCCCUGUUUGGCCUGCUGGUGUACCUGGCUGGAGCCAAGUGCACCA |
| | | CCUGUGUGGAGGAGAAGGACAGCAAGGCCAGACUGGUGCUGACCUC |
| | | UGGCAUUGUGUUUGUGAUCUCUGGCGUGCUGACCCUGAUCCCUGUG |
| | | UGCUGGACAGCCCAUGCCAUCAUCAGAGACUUCUACAACCCUCUGG |
| | | UGGCCGAGGCCCAGAAAAGAGAGCUGGGAGCCAGCCUGUACCUGGG |
| | | CUGGGCCGCCUCUGGCCUUCUUCUGCUGGGAGGAGGACUGCUGUGC |
| | | UGCACCUGCCCCUCUGGCGGCAGCCAGGGCCCCAGCCACUACAUGG |
| | | CCAGAUACAGCACCUCUGCCCCUGCCAUCAGCAGAGGCCCUUCUGA |
| | | GUACCCCACCAAGAACUAUGUGGGAGGAUCCGGUGGUGGCGGCAGC |
| | | GGCGGCAAGAAGCAGUACAUCAAGGCCAACAGCAAGUUCAUCGGCA |
| | | UCACCGAGCUGAAGAAGCUGGGAGGGGGCAAACGGGGAGGCGGCAA |
| | | AAAGAUGACCAACAGCGUGGACGACGCCCUGAUCAACAGCACCAAG |
| | | AUCUACAGCUACUUCCCCAGCGUGAUCAGCAAAGUGAACCAGGGCG |
| | | CUCAGGGCAAGAAACUGUGACUCGAGCUGGUACUGCAUGCACGCAA |
| | | UGCUAGCUGCCCCUUUCCCGUCCUGGGUACCCCGAGUCUCCCCCGA |
| | | CCUCGGGUCCCAGGUAUGCUCCCACCUCCACCUGCCCCACUCACCA |
| | | CCUCUGCUAGUUCCAGACACCUCCCAAGCACGCAGCAAUGCAGCUC |
| | | AAAACGCUUAGCCUAGCCACACCCCCACGGGAAACAGCAGUGAUUA |
| | | ACCUUUAGCAAUAAACGAAAGUUUAACUAAGCUAUACUAACCCCAG |
| | | GGUUGGUCAAUUUCGUGCCAGCCACACCGAGACCUGGUCCAGAGUC |
| | | GCUAGCCGCGUCGCUAAAAAAAAAAAAAAAAAAAAAAAAAAAAAAG |
| | | CAUAUGACUAAAAAAAAAAAAAAAAAAAAAAAAAAAAAAAAAAAAA |
| | | AAAAAAAAAAAAAAAAAAAAAAAAAAAAAAAAA |

KK-LC-1

| | | |
|---|---|---|
| 5 | KK-LC-1 | MNFYLLLASSILCALIVFWKYRRFQRNTGEMSSNSTALALVRPSSS |
| | (amino acid) | GLINSNTDNNLAVYDLSRDILNNFPHSIARQKRILVNLSMVENKLV |
| | | ELEHTLLSKGFRGASPHRKST |
| 6 | KK-LC-1 | MNFYLLLASSILCALIVFWKYRRFQRNTGEMSSNSTALALVRPSSS |
| | fusion | GLINSNTDNNLAVYDLSRDILNNFPHSIARQKRILVNLSMVENKLV |
| | (amino acid) | ELEHTLLSKGFRGASPHRKSTGGSGGGGSGGKKQYIKANSKFIGIT |
| | | ELKKLGGGKRGGGKKMTNSVDDALINSTKIYSYFPSVISKVNQGAQ |
| | | GKKL |
| 7 | KK-LC-1 (CDS) | AUGAACUUUUACCUGCUGCUUGCCAGCAGCAUUCUGUGCGCCCUGA |
| | | UUGUGUUUUGGAAAUACAGAAGAUUUCAGAGAAACACAGGAGAAAU |
| | | GUCUUCCAAUUCAACAGCUCUGGCUCUGGUGAGACCUUCUUCUUCU |
| | | GGACUGAUCAAUUCCAACACAGACAACAAUCUGGCUGUGUACGAUC |
| | | UGUCCAGAGACAUUCUGAACAAUUUUCCUCACUCAAUUGCAAGACA |
| | | GAAAAGAAUUCUGGUGAAUCUGUCAAUGGUGGAAAACAAACUGGUG |
| | | GAACUGGAACACACACUUCUGAGCAAAGGAUUCAGAGGAGCUUCUC |
| | | CUCACAGAAAAUCCACA |
| 8 | KK-LC-1 (RNA) | GGGCGAACUAGUAUUCUUCUGGUCCCCACAGACUCAGAGAGAACCC |
| | | GCCACCAUGAACUUUUACCUGCUGCUUGCCAGCAGCAUUCUGUGCG |
| | | CCCUGAUUGUGUUUUGGAAAUACAGAAGAUUUCAGAGAAACACAGG |
| | | AGAAAUGUCUUCCAAUUCAACAGCUCUGGCUCUGGUGAGACCUUCU |
| | | UCUUCUGGACUGAUCAAUUCCAACACAGACAACAAUCUGGCUGUGU |
| | | ACGAUCUGUCCAGAGACAUUCUGAACAAUUUUCCUCACUCAAUUGC |
| | | AAGACAGAAAAGAAUUCUGGUGAAUCUGUCAAUGGUGGAAAACAAA |
| | | CUGGUGGAACUGGAACACACACUUCUGAGCAAAGGAUUCAGAGGAG |
| | | CUUCUCCUCACAGAAAAUCCACAGGAGGAUCCGGUGGUGGCGGCAG |
| | | CGGCGGCAAGAAGCAGUACAUCAAGGCCAACAGCAAGUUCAUCGGC |
| | | AUCACCGAGCUGAAGAAGCUGGGAGGGGGCAAACGGGGAGGCGGCA |
| | | AAAAGAUGACCAACAGCGUGGACGACGCCCUGAUCAACAGCACCAA |
| | | GAUCUACAGCUACUUCCCCAGCGUGAUCAGCAAAGUGAACCAGGGC |
| | | GCUCAGGGCAAGAAACUGUGACUCGAGCUGGUACUGCAUGCACGCA |
| | | AUGCUAGCUGCCCCUUUCCCGUCCUGGGUACCCCGAGUCUCCCCCG |
| | | ACCUCGGGUCCCAGGUAUGCUCCCACCUCCACCUGCCCCACUCACC |
| | | ACCUCUGCUAGUUCCAGACACCUCCCAAGCACGCAGCAAUGCAGCU |
| | | CAAAACGCUUAGCCUAGCCACACCCCCACGGGAAACAGCAGUGAUU |
| | | AACCUUUAGCAAUAAACGAAAGUUUAACUAAGCUAUACUAACCCCA |
| | | GGGUUGGUCAAUUUCGUGCCAGCCACACCGAGACCUGGUCCAGAGU |
| | | CGCUAGCCGCGUCGCUAAAAAAAAAAAAAAAAAAAAAAAAAAAAAA |
| | | GCAUAUGACUAAAAAAAAAAAAAAAAAAAAAAAAAAAAAAAAAAAA |
| | | AAAAAAAAAAAAAAAAAAAAAAAAAAAAAAAAAA |

MAGEA3

| | | |
|---|---|---|
| 9 | MAGEA3 | MPLEQRSQHCKPEEGLEARGEALGLVGAQAPATEEQEAASSSSTLV |
| | (amino acid) | EVTLGEVPAAESPDPPQSPQGASSLPTTMNYPLWSQSYEDSSNQEE |
| | | EGPSTFPDLESEFQAALSRKVAELVHFLLLKYRAREPVTKAEMLGS |
| | | VVGNWQYFFPVIFSKASSSLQLVFGIELMEVDPIGHLYIFATCLGL |
| | | SYDGLLGDNQIMPKAGLLIIVLAIIAREGDCAPEEKIWEELSVLEV |
| | | FEGREDSILGDPKKLLTQHFVQENYLEYRQVPGSDPACYEFLWGPR |
| | | ALVETSYVKVLHHMVKISGGPHISYPPLHEWVLREGEE |
| 10 | MAGEA3 | MRVMAPRTLILLLSGALALTETWAGSGGSGGGGSGGMPLEQRSQHC |
| | fusion | KPEEGLEARGEALGLVGAQAPATEEQEAASSSSTLVEVTLGEVPAA |
| | (amino acid) | ESPDPPQSPQGASSLPTTMNYPLWSQSYEDSSNQEEEGPSTFPDLE |
| | | SEFQAALSRKVAELVHFLLLKYRAREPVTKAEMLGSVVGNWQYFFP |
| | | VIFSKASSSLQLVFGIELMEVDPIGHLYIFATCLGLSYDGLLGDNQ |
| | | IMPKAGLLIIVLAIIAREGDCAPEEKIWEELSVLEVFEGREDSILG |
| | | DPKKLLTQHFVQENYLEYRQVPGSDPACYEFLWGPRALVETSYVKV |
| | | LHHMVKISGGPHISYPPLHEWVLREGEEGGSGGGGSGGKKQYIKAN |
| | | SKFIGITELKKLGGGKRGGGKKMTNSVDDALINSTKIYSYFPSVIS |
| | | KVNQGAQGKKLGSSGGGGSPGGGSSIVGIVAGLAVLAVVVIGAVVA |
| | | TVMCRRKSSGGKGGSYSQAASSDSAQGSDVSLTA |
| 11 | MAGEA3 | AUGCCCCUUGAACAGCGCUCACAGCACUGCAAACCUGAGGAGGGCC |
| | (CDS) | UUGAAGCAAGGGGCGAAGCUCUGGGGUUGGUCGGUGCACAAGCACC |
| | | CGCCACUGAGGAACAGGAAGCCGCGUCUAGCUCAUCAACCCUGGUU |
| | | GAAGUGACACUGGGCGAAGUGCCUGCUGCGGAGAGUCCAGACCCUC |
| | | CCCAGUCCCCUCAAGGCGCUUCUAGCCUGCCUACCACGAUGAACUA |
| | | CCCACUGUGGUCACAGAGCUAUGAGGACAGUUCCAAUCAAGAAGAA |
| | | GAAGGCCCGUCUACCUUCCCCGAUCUUGAGUCCGAGUUUCAGGCCG |
| | | CUCUGUCCCGGAAGGUGGCAGAGCUCGUGCACUUUCUCCUGUUGAA |
| | | GUAUCGAGCCCGGGAGCCUGUCACUAAGGCCGAAAUGCUGGGCUCU |
| | | GUAGUGGGGAAUUGGCAGUAUUUCUUCCCCGUGAUCUUCAGCAAAG |
| | | CCUCCAGCAGCCUGCAAUUGGUGUUCGGUAUUGAACUGAUGGAAGU |
| | | AGAUCCGAUUGGGCAUCUGUACAUCUUUGCGACAUGUCUGGGACUG |
| | | UCCUAUGACGGACUGCUCGGGGAUAACCAGAUUAUGCCGAAAGCCG |
| | | GUCUGCUGAUCAUAGUUCUCGCCAUCAUUGCCAGAGAGGGAGAUUG |
| | | UGCUCCAGAGGAGAAGAUCUGGGAGGAAUUGUCUGUGCUGGAGGUC |
| | | UUUGAGGGUAGGGAGGACAGCAUUCUCGGCGAUCCCAAGAAACUCC |
| | | UGACCCAGCACUUUGUCCAGGAGAACUACCUCGAAUACAGACAGGU |
| | | UCCAGGCAGUGACCCUGCUUGCUACGAGUUCCUUUGGGGACCCCGU |
| | | GCAUUGGUAGAGACAAGCUAUGUCAAAGUGCUGCACCAUAUGGUGA |
| | | AGAUAUCUGGAGGACCACACAUCAGUUACCCACCCCUUCAUGAGUG |
| | | GGUUCUGCGCGAAGGGGAGGAG |
| 12 | (RNA) | GGGCGAACUAGUAUUCUUCUGGUCCCCACAGACUCAGAGAGAACCC |
| | MAGEA3 | GCCACCAUGAGAGUGAUGGCCCCCAGAACCCUGAUCCUGCUGCUGU |
| | | CUGGCGCCCUGGCCCUGACAGAGACAUGGGCCGGAAGCGGCGGCUC |
| | | UGGAGGAGGCGGCUCCGGAGGCAUGCCCCUUGAACAGCGCUCACAG |
| | | CACUGCAAACCUGAGGAGGGCCUUGAAGCAAGGGGCGAAGCUCUGG |
| | | GGUUGGUCGGUGCACAAGCACCCGCCACUGAGGAACAGGAAGCCGC |
| | | GUCUAGCUCAUCAACCCUGGUUGAAGUGACACUGGGCGAAGUGCCU |
| | | GCUGCGGAGAGUCCAGACCCUCCCCAGUCCCCUCAAGGCGCUUCUA |
| | | GCCUGCCUACCACGAUGAACUACCCACUGUGGUCACAGAGCUAUGA |
| | | GGACAGUUCCAAUCAAGAAGAAGAAGGCCCGUCUACCUUCCCCGAU |
| | | CUUGAGUCCGAGUUUCAGGCCGCUCUGUCCCGGAAGGUGGCAGAGC |
| | | UCGUGCACUUUCUCCUGUUGAAGUAUCGAGCCCGGGAGCCUGUCAC |
| | | UAAGGCCGAAAUGCUGGGCUCUGUAGUGGGGAAUUGGCAGUAUUUC |
| | | UUCCCCGUGAUCUUCAGCAAAGCCUCCAGCAGCCUGCAAUUGGUGU |
| | | UCGGUAUUGAACUGAUGGAAGUAGAUCCGAUUGGGCAUCUGUACAU |
| | | CUUUGCGACAUGUCUGGGACUGUCCUAUGACGGACUGCUCGGGGAU |
| | | AACCAGAUUAUGCCGAAAGCCGGUCUGCUGAUCAUAGUUCUCGCCA |
| | | UCAUUGCCAGAGAGGGAGAUUGUGCUCCAGAGGAGAAGAUCUGGGA |
| | | GGAAUUGUCUGUGCUGGAGGUCUUUGAGGGUAGGGAGGACAGCAUU |
| | | CUCGGCGAUCCCAAGAAACUCCUGACCCAGCACUUUGUCCAGGAGA |
| | | ACAGUUCCUUUGGGGACCCCGUGCAUUGGUAGAGACAAGCUAUGUC |
| | | AAAGUGCUGCACCAUAUGGUGAAGAUAUCUGGAGGACCACACAUCA |
| | | GUUACCUCGAAUACAGACAGGUUCCAGGCAGUGACCCUGCUUGCUA |
| | | CGUACCCACCCCUUCAUGAGUGGGUUCUGCGCGAAGGGGAGGAGGG |
| | | AGGAUCCGGUGGUGGCGGCAGCGGCGGCAAGAAGCAGUACAUCAAG |
| | | GCCAACAGCAAGUUCAUCGGCAUCACCGAGCUGAAGAAGCUGGGAG |
| | | GGGGCAAACGGGGAGGCGGCAAAAAGAUGACCAACAGCGUGGACGA |
| | | CGCCCUGAUCAACAGCACCAAGAUCUACAGCUACUUCCCCAGCGUG |
| | | AUCAGCAAAGUGAACCAGGGCGCUCAGGGCAAGAAACUGGGCUCUA |
| | | GCGGAGGGGGAGGCUCUCCUGGCGGGGGAUCUAGCAUCGUGGGAAU |
| | | UGUGGCAGGACUGGCAGUGCUGGCCGUGGUGGUGAUCGGAGCCGUG |
| | | GUGGCUACCGUGAUGUGCAGACGGAAGUCCAGCGGAGGCAAGGGCG |
| | | GCAGCUACAGCCAGGCCGCCAGCUCUGAUAGCGCCCAGGGCAGCGA |
| | | CGUGUCACUGACAGCCUAGUAACUCGAGCUGGUACUGCAUGCACGC |
| | | AAUGCUAGCUGCCCCUUUCCCGUCCUGGGUACCCCGAGUCUCCCCC |
| | | GACCUCGGGUCCCAGGUAUGCUCCCACCUCCACCUGCCCCACUCAC |
| | | CACCUCUGCUAGUUCCAGACACCUCCCAAGCACGCAGCAAUGCAGC |
| | | UCAAAACGCUUAGCCUAGCCACACCCCCACGGGAAACAGCAGUGAU |
| | | UAACCUUUAGCAAUAAACGAAAGUUUAACUAAGCUAUACUAACCCC |
| | | AGGGUUGGUCAAUUUCGUGCCAGCCACACCGAGACCUGGUCCAGAG |
| | | UCGCUAGCCGCGUCGCUAAAAAAAAAAAAAAAAAAAAAAAAAAAAA |
| | | AGCAUAUGACUAAAAAAAAAAAAAAAAAAAAAAAAAAAAAAAAAAA |
| | | AAAAAAAAAAAAAAAAAAAAAAAAAAAAAAAAAAA |

MAGEA4

| | | |
|---|---|---|
| 13 | MAGEA4 | MLPLSVGLWVPIAQLLPALLPAALTRVIMSSEQKSQHCKPEEGVEA |
| | (amino acid) | QEEALGLVGAQAPTTEEQEAAVSSSSPLVPGTLEEVPAAESAGPPQ |
| | | SPQGASALPTTISFTCWRQPNEGSSSQEEEGPSTSPDAESLFREAL |
| | | SNKVDELAHFLLRKYRAKELVTKAEMLERVIKNYKRCFPVIFGKAS |
| | | ESLKMIFGIDVKEVDPASNTYTLVTCLGLSYDGLLGNNQIFPKTGL |
| | | LIIVLGTIAMEGDSASEEEIWEELGVMGVYDGREHTVYGEPRKLLT |
| | | QDWVQENYLEYRQVPGSNPARYEFLWGPRALAETSYVKVLEHVVRV |
| | | NARVRIAYPSLREAALLEEEEGV |
| 14 | MAGEA4 | MLPLSVGLWVPIAQLLPALLPAALTRVIMSSEQKSQHCKPEEGVEA |
| | fusion | QEEALGLVGAQAPTTEEQEAAVSSSSPLVPGTLEEVPAAESAGPPQ |
| | (amino acid) | SPQGASALPTTISFTCWRQPNEGSSSQEEEGPSTSPDAESLFREAL |
| | | SNKVDELAHFLLRKYRAKELVTKAEMLERVIKNYKRCFPVIFGKAS |
| | | ESLKMIFGIDVKEVDPASNTYTLVTCLGLSYDGLLGNNQIFPKTGL |
| | | LIIVLGTIAMEGDSASEEEIWEELGVMGVYDGREHTVYGEPRKLLT |
| | | QDWVQENYLEYRQVPGSNPARYEFLWGPRALAETSYVKVLEHVVRV |
| | | NARVRIAYPSLREAALLEEEEGVGGSGGGGSGGKKQYIKANSKFIG |
| | | ITELKKLGGGKRGGGKKMTNSVDDALINSTKIYSYFPSVISKVNQG |
| | | AQGKKLGSSGGGGSPGGGSSIVGIVAGLAVLAVVVIGAVVATVMCR |
| | | RKSSGGKGGSYSQAASSDSAQGSDVSLTA |
| 15 | MAGEA4 | AUGCUGCCUCUGUCUGUGGGCCUGUGGGUGCCAAUUGCCCAGCUGC |
| | (CDS) | UGCCUGCCCUGCUGCCUGCUGCCCUGACCAGAGUGAUCAUGUCUUC |
| | | UGAGCAGAAGUCUCAGCACUGCAAGCCUGAGGAGGGAGUGGAGGCC |
| | | CAGGAGGAGGCCCUGGGCCUGGUGGGAGCCCAGGCCCCAACAACAG |
| | | AGGAGCAGGAGGCUGCUGUGAGCAGCAGCAGCCCUCUGGUGCCUGG |
| | | CACACUGGAGGAGGUGCCUGCUGCUGAGUCUGCUGGACCUCCUCAG |
| | | UCUCCUCAGGGAGCCUCUGCCCUGCCUACAACAAUCAGCUUCACAU |
| | | GCUGGAGACAGCCCAAUGAGGGCAGCAGCAGCCAGGAGGAGGAGGG |
| | | CCCAAGCACCUCUCCUGAUGCCGAGAGCCUGUUCAGAGAGGCCCUG |
| | | AGCAACAAGGUGGAUGAGCUGGCCCACUUUCUGCUGAGAAAGUACA |
| | | GAGCCAAGGAGCUGGUGACAAAGGCUGAAAUGCUGGAAAGAGUGAU |
| | | CAAGAAUUACAAGAGAUGCUUUCCUGUGAUCUUUGGCAAAGCCUCU |
| | | GAAUCUCUGAAGAUGAUCUUUGGCAUUGAUGUGAAGGAAGUGGACC |
| | | CUGCCAGCAACACCUACACCCUGGUGACCUGCCUGGGCCUGAGCUA |
| | | UGAUGGCCUGCUGGGCAACAAUCAGAUCUUUCCCAAGACAGGCCUG |
| | | CUGAUCAUUGUGCUGGGCACAAUUGCCAUGGAGGGAGAUUCUGCCU |
| | | CUGAGGAGGAGAUCUGGGAGGAGCUGGGAGUGAUGGGAGUGUAUGA |
| | | UGGCAGAGAACACACAGUGUAUGGAGAACCCAGAAAACUGCUGACC |
| | | CAGGAUUGGGUGCAGGAAAAUUACCUGGAGUACAGACAGGUGCCUG |
| | | GCAGCAAUCCUGCCAGAUAUGAGUUCCUGUGGGGACCAAGAGCUCU |
| | | GGCUGAAACAUCUUAUGUGAAAGUGCUGGAGCAUGUGGUGAGAGUG |
| | | AAUGCCAGAGUGAGAAUUGCCUACCCUUCUCUGAGAGAGGCUGCUC |
| | | UGCUGGAGGAGGAGGAGGGAGUG |
| 16 | MAGEA4 | GGGCGAACUAGUAUUCUUCUGGUCCCCACAGACUCAGAGAGAACCC |
| | (RNA) | GCCACCAUGCUGCCUCUGUCUGUGGGCCUGUGGGUGCCAAUUGCCC |
| | | AGCUGCUGCCUGCCCUGCUGCCUGCUGCCCUGACCAGAGUGAUCAU |
| | | GUCUUCUGAGCAGAAGUCUCAGCACUGCAAGCCUGAGGAGGGAGUG |
| | | GAGGCCCAGGAGGAGGCCCUGGGCCUGGUGGGAGCCCAGGCCCCAA |
| | | CAACAGAGGAGCAGGAGGCUGCUGUGAGCAGCAGCAGCCCUCUGGU |
| | | GCCUGGCACACUGGAGGAGGUGCCUGCUGCUGAGUCUGCUGGACCU |
| | | CCUCAGUCUCCUCAGGGAGCCUCUGCCCUGCCUACAACAAUCAGCU |
| | | UCACAUGCUGGAGACAGCCCAAUGAGGGCAGCAGCAGCCAGGAGGA |
| | | GGAGGGCCCAAGCACCUCUCCUGAUGCCGAGAGCCUGUUCAGAGAG |
| | | GCCCUGAGCAACAAGGUGGAUGAGCUGGCCCACUUUCUGCUGAGAA |
| | | AGUACAGAGCCAAGGAGCUGGUGACAAAGGCUGAAAUGCUGGAAAG |
| | | AGUGAUCAAGAAUUACAAGAGAUGCUUUCCUGUGAUCUUUGGCAAA |
| | | GCCUCUGAAUCUCUGAAGAUGAUCUUUGGCAUUGAUGUGAAGGAAG |
| | | UGGACCCUGCCAGCAACACCUACACCCUGGUGACCUGCCUGGGCCU |
| | | GAGCUAUGAUGGCCUGCUGGGCAACAAUCAGAUCUUUCCCAAGACA |
| | | GGCCUGCUGAUCAUUGUGCUGGGCACAAUUGCCAUGGAGGGAGAUU |
| | | CUGCCUCUGAGGAGGAGAUCUGGGAGGAGCUGGGAGUGAUGGGAGU |
| | | GUAUGAUGGCAGAGAACACACAGUGUAUGGAGAACCCAGAAAACUG |
| | | CUGACCCAGGAUUGGGUGCAGGAAAAUUACCUGGAGUACAGACAGG |
| | | UGCCUGGCAGCAAUCCUGCCAGAUAUGAGUUCCUGUGGGGACCAAG |
| | | AGCUCUGGCUGAAACAUCUUAUGUGAAAGUGCUGGAGCAUGUGGUG |
| | | AGAGUGAAUGCCAGAGUGAGAAUUGCCUACCCUUCUCUGAGAGAGG |
| | | CUGCUCUGCUGGAGGAGGAGGAGGGAGUGGGAGGAUCCGGUGGUGG |
| | | CGGCAGCGGCGGCAAGAAGCAGUACAUCAAGGCCAACAGCAAGUUC |
| | | AUCGGCAUCACCGAGCUGAAGAAGCUGGGAGGGGGCAAACGGGGAG |
| | | GCGGCAAAAAGAUGACCAACAGCGUGGACGACGCCCUGAUCAACAG |
| | | CACCAAGAUCUACAGCUACUUCCCCAGCGUGAUCAGCAAAGUGAAC |
| | | CAGGGCGCUCAGGGCAAGAAACUGGGCUCUAGCGGAGGGGGAGGCU |
| | | CUCCUGGCGGGGGAUCUAGCAUCGUGGGAAUUGUGGCAGGACUGGC |
| | | AGUGCUGGCCGUGGUGGUGAUCGGAGCCGUGGUGGCUACCGUGAUG |
| | | UGCAGACGGAAGUCCAGCGGAGGCAAGGGCGGCAGCUACAGCCAGG |
| | | CCGCCAGCUCUGAUAGCGCCCAGGGCAGCGACGUGUCACUGACAGC |
| | | CUAGUAACUCGAGCUGGUACUGCAUGCACGCAAUGCUAGCUGCCCC |
| | | UUUCCCGUCCUGGGUACCCCGAGUCUCCCCCGACCUCGGGUCCCAG |
| | | GUAUGCUCCCACCUCCACCUGCCCCACUCACCACCUCUGCUAGUUC |
| | | CAGACACCUCCCAAGCACGCAGCAAUGCAGCUCAAAACGCUUAGCC |
| | | UAGCCACACCCCCACGGGAAACAGCAGUGAUUAACCUUUAGCAAUA |
| | | AACGAAAGUUUAACUAAGCUAUACUAACCCCAGGGUUGGUCAAUUU |
| | | CGUGCCAGCCACACCGAGACCUGGUCCAGAGUCGCUAGCCGCGUCG |
| | | CUAAAAAAAAAAAAAAAAAAAAAAAAAAAAAAGCAUAUGACUAAAA |
| | | AAAAAAAAAAAAAAAAAAAAAAAAAAAAAAAAAAAAAAAAAAAAAA |
| | | AAAAAAAAAAAAAAAAAAAA |

PRAME

| | | |
|---|---|---|
| 17 | PRAME | MERRRLWGSIQSRYISMSVWTSPRRLVELAGQSLLKDEALAIAALE |
| | (amino acid) | LLPRELFPPLFMAAFDGRHSQTLKAMVQAWPFTCLPLGVLMKGQHL |
| | | HLETFKAVLDGLDVLLAQEVRPRRWKLQVLDLRKNSHQDFWTVWSG |
| | | NRASLYSFPEPEAAQPMTKKRKVDGLSTEAEQPFIPVEVLVDLFLK |
| | | EGACDELFSYLIEKVKRKKNVLRLCCKKLKIFAMPMQDIKMILKMV |
| | | QLDSIEDLEVTCTWKLPTLAKFSPYLGQMINLRRLLLSHIHASSYI |
| | | SPEKEEQYIAQFTSQFLSLQCLQALYVDSLFFLRGRLDQLLRHVMN |
| | | PLETLSITNCRLSEGDVMHLSQSPSVSQLSVLSLSGVMLTDVSPEP |
| | | LQALLERASATLQDLVFDECGITDDQLLALLPSLSHCSQLTTLSFY |
| | | GNSISISALQSLLQHLIGLSNLTHVLYPVPLESYEDIHGTLHLERL |
| | | AYLHARLRELLCELGRPSMVWLSANPCPHCGDRTFYDPEPILCPCF |
| | | MPN |
| 18 | PRAME fusion | MRVMAPRTLILLLSGALALTETWAGSGGSGGGGSGGMERRRLWGSI |
| | (amino acid) | QSRYISMSVWTSPRRLVELAGQSLLKDEALAIAALELLPRELFPPL |
| | | FMAAFDGRHSQTLKAMVQAWPFTCLPLGVLMKGQHLHLETFKAVLD |
| | | GLDVLLAQEVRPRRWKLQVLDLRKNSHQDFWTVWSGNRASLYSFPE |
| | | PEAAQPMTKKRKVDGLSTEAEQPFIPVEVLVDLFLKEGACDELFSY |
| | | LIEKVKRKKNVLRLCCKKLKIFAMPMQDIKMILKMVQLDSIEDLEV |
| | | TCTWKLPTLAKFSPYLGQMINLRRLLLSHIHASSYISPEKEEQYIA |
| | | QFTSQFLSLQCLQALYVDSLFFLRGRLDQLLRHVMNPLETLSITNC |
| | | RLSEGDVMHLSQSPSVSQLSVLSLSGVMLTDVSPEPLQALLERASA |
| | | TLQDLVFDECGITDDQLLALLPSLSHCSQLTTLSFYGNSISISALQ |
| | | SLLQHLIGLSNLTHVLYPVPLESYEDIHGTLHLERLAYLHARLREL |
| | | LCELGRPSMVWLSANPCPHCGDRTFYDPEPILCPCFMPNGGSGGGG |
| | | SGGKKQYIKANSKFIGITELKKLGGGKRGGGKKMTNSVDDALINST |
| | | KIYSYFPSVISKVNQGAQGKKLGSSGGGGSPGGGSSIVGIVAGLAV |
| | | LAVVVIGAVVATVMCRRKSSGGKGGSYSQAASSDSAQGSDVSLTA |
| 19 | PRAME (CDS) | AUGGAACGAAGGCGUUUGUGGGGUUCCAUUCAGAGCCGAUACAUC |
| | | AGCAUGAGUGUGUGGACAAGCCCACGGAGACUUGUGGAGCUGGCAG |
| | | GGCAGAGCCUGCUGAAGGAUGAGGCCCUGGCCAUUGCCGCCCUGGA |
| | | GUUGCUGCCCAGGGAGCUGUUCCCGCCACUGUUCAUGGCAGCCUUU |
| | | GACGGGAGACACAGCCAGACCCUGAAGGCAAUGGUGCAGGCCUGGC |
| | | CCUUCACCUGCCUCCCUCUGGGAGUGCUGAUGAAGGGACAACAUCU |
| | | UCACCUGGAGACCUUCAAAGCUGUGCUUGAUGGACUUGAUGUGCUC |
| | | CUUGCCCAGGAGGUUCGCCCCAGGAGGUGGAAACUUCAAGUGCUGG |
| | | AUUUACGGAAGAACUCUCAUCAGGACUUCUGGACUGUAUGGUCUGG |
| | | AAACAGGGCCAGUCUGUACUCAUUUCCAGAGCCAGAAGCAGCUCAG |
| | | CCCAUGACAAAGAAGCGAAAAGUAGAUGGUUUGAGCACAGAGGCAG |
| | | AGCAGCCCUUCAUUCCAGUAGAGGUGCUCGUAGACCUGUUCCUCAA |
| | | GGAAGGUGCCUGUGAUGAAUUGUUCUCCUACCUCAUUGAGAAAGUG |
| | | AAGCGAAAGAAAAAUGUACUACGCCUGUGCUGUAAGAAGCUGAAGA |
| | | UUUUUGCAAUGCCCAUGCAGGAUAUCAAGAUGAUCCUGAAAAUGGU |
| | | GCAGCUGGACUCUAUUGAAGAUUUGGAAGUGACUUGUACCUGGAAG |
| | | CUACCCACCUUGGCGAAAUUUUCUCCUUACCUGGGCCAGAUGAUUA |
| | | AUCUGCGUAGACUCCUCCUCUCCCACAUCCAUGCAUCUUCCUACAU |
| | | UUCCCCGGAGAAGGAGGAACAGUAUAUCGCCCAGUUCACCUCUCAG |
| | | UUCCUCAGUCUGCAGUGCCUCCAGGCUCUCUAUGUGGACUCUUUAU |
| | | UUUUCCUUAGAGGCCGCCUGGAUCAGUUGCUCAGGCACGUGAUGAA |
| | | CCCCUUGGAAACCCUCUCAAUAACUAACUGCCGGCUUUCGGAAGGG |
| | | GAUGUGAUGCAUCUGUCCCAGAGUCCCAGCGUCAGUCAGCUAAGUG |
| | | UCCUGAGUCUAAGUGGGGUCAUGCUGACCGAUGUAAGUCCCGAGCC |
| | | CCUCCAAGCUCUGCUGGAGAGAGCCUCUGCCACCCUCCAGGACCUG |
| | | GUCUUUGAUGAGUGUGGGAUCACGGAUGAUCAGCUCCUUGCCCUCC |
| | | UGCCUUCCCUGAGCCACUGCUCCCAGCUUACAACCUUAAGCUUCUA |
| | | CGGGAAUUCCAUCUCCAUAUCUGCCUUGCAGAGUCUCCUGCAGCAC |
| | | CUCAUCGGGCUGAGCAAUCUGACCCACGUGCUGUAUCCUGUCCCCC |
| | | UGGAGAGUUAUGAGGACAUCCAUGGUACCCUCCACCUGGAGAGGCU |
| | | UGCCUAUCUGCAUGCCAGGCUCAGGGAGUUGCUGUGUGAGUUGGGG |
| | | CGGCCCAGCAUGGUCUGGCUUAGUGCCAACCCCUGUCCUCACUGUG |
| | | GGGACAGAACCUUCUAUGACCCGGAGCCCAUCCUGUGCCCCUGUUU |
| | | CAUGCCUAAC |
| 20 | PRAME (RNA) | GGGCGAACUAGUAUUCUUCUGGUCCCCACAGACUCAGAGAGAACCC |
| | | GCCACCAUGAGAGUGAUGGCCCCCAGAACCCUGAUCCUGCUGCUGU |
| | | CUGGCGCCCUGGCCCUGACAGAGACAUGGGCCGGAAGCGGCGGCUC |
| | | UGGAGGAGGCGGCUCCGGAGGCAUGGAACGAAGGCGUUUGUGGGGU |
| | | UCCAUUCAGAGCCGAUACAUCAGCAUGAGUGUGUGGACAAGCCCAC |
| | | GGAGACUUGUGGAGCUGGCAGGGCAGAGCCUGCUGAAGGAUGAGGC |
| | | CCUGGCCAUUGCCGCCCUGGAGUUGCUGCCCAGGGAGCUGUUCCCG |
| | | CCACUGUUCAUGGCAGCCUUUGACGGGAGACACAGCCAGACCCUGA |
| | | AGGCAAUGGUGCAGGCCUGGCCCUUCACCUGCCUCCCUCUGGGAGU |
| | | GCUGAUGAAGGGACAACAUCUUCACCUGGAGACCUUCAAAGCUGUG |
| | | CUUGAUGGACUUGAUGUGCUCCUUGCCCAGGAGGUUCGCCCCAGGA |
| | | GGUGGAAACUUCAAGUGCUGGAUUUACGGAAGAACUCUCAUCAGGA |
| | | CUUCUGGACUGUAUGGUCUGGAAACAGGGCCAGUCUGUACUCAUUU |
| | | CCAGAGCCAGAAGCAGCUCAGCCCAUGACAAAGAAGCGAAAAGUAG |
| | | AUGGUUUGAGCACAGAGGCAGAGCAGCCCUUCAUUCCAGUAGAGGU |
| | | GCUCGUAGACCUGUUCCUCAAGGAAGGUGCCUGUGAUGAAUUGUUC |
| | | UCCUACCUCAUUGAGAAAGUGAAGCGAAAGAAAAAUGUACUACGCC |
| | | UGUGCUGUAAGAAGCUGAAGAUUUUUGCAAUGCCCAUGCAGGAUAU |
| | | CAAGAUGAUCCUGAAAAUGGUGCAGCUGGACUCUAUUGAAGAUUUG |
| | | GAAGUGACUUGUACCUGGAAGCUACCCACCUUGGCGAAAUUUUCUC |
| | | CUUACCUGGGCCAGAUGAUUAAUCUGCGUAGACUCCUCCUCUCCCA |
| | | CAUCCAUGCAUCUUCCUACAUUUCCCCGGAGAAGGAGGAACAGUAU |
| | | AUCGCCCAGUUCACCUCUCAGUUCCUCAGUCUGCAGUGCCUCCAGG |
| | | CUCUCUAUGUGGACUCUUUAUUUUUCCUUAGAGGCCGCCUGGAUCA |
| | | GUUGCUCAGGCACGUGAUGAACCCCUUGGAAACCCUCUCAAUAACU |
| | | AACUGCCGGCUUUCGGAAGGGGAUGUGAUGCAUCUGUCCCAGAGUC |
| | | CCAGCGUCAGUCAGCUAAGUGUCCUGAGUCUAAGUGGGGUCAUGCU |
| | | GACCGAUGUAAGUCCCGAGCCCCUCCAAGCUCUGCUGGAGAGAGCC |
| | | UCUGCCACCCUCCAGGACCUGGUCUUUGAUGAGUGUGGGAUCACGG |
| | | AUGAUCAGCUCCUUGCCCUCCUGCCUUCCCUGAGCCACUGCUCCCA |
| | | GCUUACAACCUUAAGCUUCUACGGGAAUUCCAUCUCCAUAUCUGCC |
| | | UUGCAGAGUCUCCUGCAGCACCUCAUCGGGCUGAGCAAUCUGACCC |
| | | ACGUGCUGUAUCCUGUCCCCCUGGAGAGUUAUGAGGACAUCCAUGG |
| | | UACCCUCCACCUGGAGAGGCUUGCCUAUCUGCAUGCCAGGCUCAGG |
| | | GAGUUGCUGUGUGAGUUGGGGCGGCCCAGCAUGGUCUGGCUUAGUG |
| | | CCAACCCCUGUCCUCACUGUGGGGACAGAACCUUCUAUGACCCGGA |
| | | GCCCAUCCUGUGCCCCUGUUUCAUGCCUAACGGAGGAUCCGGUGGU |
| | | GGCGGCAGCGGCGGCAAGAAGCAGUACAUCAAGGCCAACAGCAAGU |
| | | UCAUCGGCAUCACCGAGCUGAAGAAGCUGGGAGGGGGCAAACGGGG |
| | | AGGCGGCAAAAAGAUGACCAACAGCGUGGACGACGCCCUGAUCAAC |
| | | AGCACCAAGAUCUACAGCUACUUCCCCAGCGUGAUCAGCAAAGUGA |
| | | ACCAGGGCGCUCAGGGCAAGAAACUGGGCUCUAGCGGAGGGGGAGG |
| | | CUCUCCUGGCGGGGGAUCUAGCAUCGUGGGAAUUGUGGCAGGACUG |
| | | GCAGUGCUGGCCGUGGUGGUGAUCGGAGCCGUGGUGGCUACCGUGA |
| | | UGUGCAGACGGAAGUCCAGCGGAGGCAAGGGCGGCAGCUACAGCCA |
| | | GGCCGCCAGCUCUGAUAGCGCCCAGGGCAGCGACGUGUCACUGACA |
| | | GCCUAGUAACUCGAGCUGGUACUGCAUGCACGCAAUGCUAGCUGCC |
| | | CCUUUCCCGUCCUGGGUACCCCGAGUCUCCCCCGACCUCGGGUCCC |
| | | AGGUAUGCUCCCACCUCCACCUGCCCCACUCACCACCUCUGCUAGU |
| | | UCCAGACACCUCCCAAGCACGCAGCAAUGCAGCUCAAAACGCUUAG |
| | | CCUAGCCACACCCCCACGGGAAACAGCAGUGAUUAACCUUUAGCAA |
| | | UAAACGAAAGUUUAACUAAGCUAUACUAACCCCAGGGUUGGUCAAU |
| | | UUCGUGCCAGCCACACCGAGACCUGGUCCAGAGUCGCUAGCCGCGU |
| | | CGCUAAAAAAAAAAAAAAAAAAAAAAAAAAAAAAGCAUAUGACUAA |
| | | AAAAAAAAAAAAAAAAAAAAAAAAAAAAAAAAAAAAAAAAAAAAAA |
| | | AAAAAAAAAAAAAAAAAAAAAA |

MAGEC1

| | | |
|---|---|---|
| 21 | MAGEC1 | MLTNVISRYTGYFPVIFRKAREFIEILFGISLREVDPDDSYVFVNT |
| | (amino acid) | LDLTSEGCLSDEQGMSQNRLLILILSIIFIKGTYASEEVIWDVLSG |
| | | IGVRAGREHFAFGEPRELLTKVWVQEHYLEYREVPNSSPPRYEFLW |
| | | GPRAHSEVIKRKVVEFLAMLKNTVPITFPSSYKDALKDVEERAQAI |
| | | IDTTDDSTATESASSSVMSPSFSSE |
| 22 | MAGEC1 | MRVMAPRTLILLLSGALALTETWAGSGGSGGGGSGGMLTNVISRYT |
| | fusion | GYFPVIFRKAREFIEILFGISLREVDPDDSYVFVNTLDLTSEGCLS |
| | (amino acid) | DEQGMSQNRLLILILSIIFIKGTYASEEVIWDVLSGIGVRAGREHF |
| | | AFGEPRELLTKVWVQEHYLEYREVPNSSPPRYEFLWGPRAHSEVIK |
| | | RKVVEFLAMLKNTVPITFPSSYKDALKDVEERAQAIIDTTDDSTAT |
| | | ESASSSVMSPSFSSEGGSGGGGSGGKKQYIKANSKFIGITELKKLG |
| | | GGKRGGGKKMTNSVDDALINSTKIYSYFPSVISKVNQGAQGKKLGS |
| | | SGGGGSPGGGSSIVGIVAGLAVLAVVVIGAVVATVMCRRKSSGGKG |
| | | GSYSQAASSDSAQGSDVSLTA |
| 23 | MAGEC1 | AUGCUGACGAAUGUCAUCAGCAGGUACACGGGCUACUUUCCUGUGA |
| | (CDS) | UCUUCAGGAAAGCCCGUGAGUUCAUAGAGAUACUUUUUGGCAUUUC |
| | | CCUGAGAGAAGUGGACCCUGAUGACUCCUAUGUCUUUGUAAACACA |
| | | UUAGACCUCACCUCUGAGGGGUGUCUGAGUGAUGAGCAGGGCAUGU |
| | | CCCAGAACCGCCUCCUGAUUCUUAUUCUGAGUAUCAUCUUCAUAAA |
| | | GGGCACCUAUGCCUCUGAGGAGGUCAUCUGGGAUGUGCUGAGUGGA |
| | | AUAGGGGUGCGUGCUGGGAGGGAGCACUUUGCCUUUGGGGAGCCCA |
| | | GGGAGCUCCUCACUAAAGUUUGGGUGCAGGAACAUUACCUAGAGUA |
| | | CCGGGAGGUGCCCAAUUCUUCUCCUCCUCGUUACGAAUUCCUGUGG |
| | | GGUCCAAGAGCUCAUUCAGAAGUCAUUAAGAGGAAAGUAGUAGAGU |
| | | UUUUGGCCAUGCUAAAGAAUACCGUCCCUAUUACCUUUCCAUCCUC |
| | | UUACAAGGAUGCUUUGAAAGAUGUGGAGGAGAGAGCCCAGGCCAUA |
| | | AUUGACACCACAGAUGAUUCGACUGCCACAGAAAGUGCAAGCUCCA |
| | | GUGUCAUGUCCCCCAGCUUUUCUUCUGAG |
| 24 | MAGEC1 | GGGCGAACUAGUAUUCUUCUGGUCCCCACAGACUCAGAGAGAACCC |
| | (RNA) | GCCACCAUGAGAGUGAUGGCCCCCAGAACCCUGAUCCUGCUGCUGU |
| | | CUGGCGCCCUGGCCCUGACAGAGACAUGGGCCGGAAGCGGCGGCUC |
| | | UGGAGGAGGCGGCUCCGGAGGCAUGCUGACGAAUGUCAUCAGCAGG |
| | | UACACGGGCUACUUUCCUGUGAUCUUCAGGAAAGCCCGUGAGUUCA |
| | | UAGAGAUACUUUUUGGCAUUUCCCUGAGAGAAGUGGACCCUGAUGA |
| | | CUCCUAUGUCUUUGUAAACACAUUAGACCUCACCUCUGAGGGGUGU |
| | | CUGAGUGAUGAGCAGGGCAUGUCCCAGAACCGCCUCCUGAUUCUUA |
| | | UUCUGAGUAUCAUCUUCAUAAAGGGCACCUAUGCCUCUGAGGAGGU |
| | | CAUCUGGGAUGUGCUGAGUGGAAUAGGGGUGCGUGCUGGGAGGGAG |
| | | CACUUUGCCUUUGGGGAGCCCAGGGAGCUCCUCACUAAAGUUUGGG |
| | | UGCAGGAACAUUACCUAGAGUACCGGGAGGUGCCCAAUUCUUCUCC |
| | | UCCUCGUUACGAAUUCCUGUGGGGUCCAAGAGCUCAUUCAGAAGUC |
| | | AUUAAGAGGAAAGUAGUAGAGUUUUUGGCCAUGCUAAAGAAUACCG |
| | | UCCCUAUUACCUUUCCAUCCUCUUACAAGGAUGCUUUGAAAGAUGU |
| | | GGAGGAGAGAGCCCAGGCCAUAAUUGACACCACAGAUGAUUCGACU |
| | | GCCACAGAAAGUGCAAGCUCCAGUGUCAUGUCCCCCAGCUUUUCUU |
| | | CUGAGGGAGGAUCCGGUGGUGGCGGCAGCGGCGGCAAGAAGCAGUA |
| | | CAUCAAGGCCAACAGCAAGUUCAUCGGCAUCACCGAGCUGAAGAAG |
| | | CUGGGAGGGGGCAAACGGGGAGGCGGCAAAAAGAUGACCAACAGCG |
| | | UGGACGACGCCCUGAUCAACAGCACCAAGAUCUACAGCUACUUCCC |
| | | CAGCGUGAUCAGCAAAGUGAACCAGGGCGCUCAGGGCAAGAAACUG |
| | | GGCUCUAGCGGAGGGGGAGGCUCUCCUGGCGGGGGAUCUAGCAUCG |
| | | UGGGAAUUGUGGCAGGACUGGCAGUGCUGGCCGUGGUGGUGAUCGG |
| | | AGCCGUGGUGGCUACCGUGAUGUGCAGACGGAAGUCCAGCGGAGGC |
| | | AAGGGCGGCAGCUACAGCCAGGCCGCCAGCUCUGAUAGCGCCCAGG |
| | | GCAGCGACGUGUCACUGACAGCCUAGUAACUCGAGCUGGUACUGCA |
| | | UGCACGCAAUGCUAGCUGCCCCUUUCCCGUCCUGGGUACCCCGAGU |
| | | CUCCCCCGACCUCGGGUCCCAGGUAUGCUCCCACCUCCACCUGCCC |
| | | CACUCACCACCUCUGCUAGUUCCAGACACCUCCCAAGCACGCAGCA |
| | | AUGCAGCUCAAAACGCUUAGCCUAGCCACACCCCCACGGGAAACAG |
| | | CAGUGAUUAACCUUUAGCAAUAAACGAAAGUUUAACUAAGCUAUAC |
| | | UAACCCCAGGGUUGGUCAAUUUCGUGCCAGCCACACCGAGACCUGG |
| | | UCCAGAGUCGCUAGCCGCGUCGCUAAAAAAAAAAAAAAAAAAAAAA |
| | | AAAAAAAAGCAUAUGACUAAAAAAAAAAAAAAAAAAAAAAAAAAAA |
| | | AAAAAAAAAAAAAAAAAAAAAAAAAAAAAAAAAAAAAAAAAA |

NY-ESO-1

| | | |
|---|---|---|
| 25 | NY-ESO-1 | MQAEGRGTGGSTGDADGPGGPGIPDGPGGNAGGPGEAGATGGRGPR |
| | (amino acid) | GAGAARASGPGGGAPRGPHGGAASGLNGCCRCGARGPESRLLEFYL |
| | | AMPFATPMEAELARRSLAQDAPPLPVPGVLLKEFTVSGNILTIRLT |
| | | AADHRQLQLSISSCLQQLSLLMWITQCFLPVFLAQPPSGQRR |
| 26 | NY-ESO-1 | MRVMAPRTLILLLSGALALTETWAGSGGSGGGGSGGMQAEGRGTGG |
| | fusion | STGDADGPGGPGIPDGPGGNAGGPGEAGATGGRGPRGAGAARASGP |
| | (amino acid) | GGGAPRGPHGGAASGLNGCCRCGARGPESRLLEFYLAMPFATPMEA |
| | | ELARRSLAQDAPPLPVPGVLLKEFTVSGNILTIRLTAADHRQLQLS |
| | | ISSCLQQLSLLMWITQCFLPVFLAQPPSGQRRGGSGGGGSGGKKQY |
| | | IKANSKFIGITELKKLGGGKRGGGKKMTNSVDDALINSTKIYSYFP |
| | | SVISKVNQGAQGKKLGSSGGGGSPGGGSSIVGIVAGLAVLAVVVIG |
| | | AVVATVMCRRKSSGGKGGSYSQAASSDSAQGSDVSLTA |
| 27 | NY-ESO-1 | AUGCAGGCCGAGGGCAGAGGAACAGGCGGCAGCACAGGCGACGCAG |
| | (CDS) | AUGGACCAGGCGGCCCUGGAAUCCCUGAUGGCCCAGGCGGCAAUGC |
| | | UGGGGGACCAGGAGAAGCUGGCGCCACAGGCGGGAGAGGACCUAGA |
| | | GGAGCUGGAGCCGCUAGAGCUUCUGGACCUGGGGGAGGCGCCCCUA |
| | | GAGGACCACAUGGAGGCGCUGCCAGCGGCCUGAAUGGCUGCUGCAG |
| | | AUGCGGCGCCAGAGGCCCUGAGAGCCGGCUGCUGGAAUUCUACCUG |
| | | GCCAUGCCCUUCGCCACCCCCAUGGAAGCCGAGCUGGCCAGAAGAU |
| | | CCCUGGCUCAGGACGCUCCUCCUCUGCCUGUGCCCGGCGUGCUGCU |
| | | GAAAGAAUUCACCGUGUCCGGCAACAUCCUGACCAUCAGACUGACA |
| | | GCCGCCGAUCACAGACAGCUCCAGCUGAGCAUCAGCUCUUGCCUGC |
| | | AGCAGCUGAGCCUGCUGAUGUGGAUCACCCAGUGCUUUCUGCCCGU |
| | | GUUCCUGGCCCAGCCACCCAGCGGACAGAGAAGG |
| 28 | NY-ESO-1 | GGGCGAACUAGUAUUCUUCUGGUCCCCACAGACUCAGAGAGAACCC |
| | (RNA) | GCCACCAUGAGAGUGAUGGCCCCCAGAACCCUGAUCCUGCUGCUGU |
| | | CUGGCGCCCUGGCCCUGACAGAGACAUGGGCCGGAAGCGGCGGCUC |
| | | UGGAGGAGGCGGCUCCGGAGGCAUGCAGGCCGAGGGCAGAGGAACA |
| | | GGCGGCAGCACAGGCGACGCAGAUGGACCAGGCGGCCCUGGAAUCC |
| | | CUGAUGGCCCAGGCGGCAAUGCUGGGGGACCAGGAGAAGCUGGCGC |
| | | CACAGGCGGGAGAGGACCUAGAGGAGCUGGAGCCGCUAGAGCUUCU |
| | | GGACCUGGGGGAGGCGCCCCUAGAGGACCACAUGGAGGCGCUGCCA |
| | | GCGGCCUGAAUGGCUGCUGCAGAUGCGGCGCCAGAGGCCCUGAGAG |
| | | CCGGCUGCUGGAAUUCUACCUGGCCAUGCCCUUCGCCACCCCCAUG |
| | | GAAGCCGAGCUGGCCAGAAGAUCCCUGGCUCAGGACGCUCCUCCUC |
| | | UGCCUGUGCCCGGCGUGCUGCUGAAAGAAUUCACCGUGUCCGGCAA |
| | | CAUCCUGACCAUCAGACUGACAGCCGCCGAUCACAGACAGCUCCAG |
| | | CUGAGCAUCAGCUCUUGCCUGCAGCAGCUGAGCCUGCUGAUGUGGA |
| | | UCACCCAGUGCUUUCUGCCCGUGUUCCUGGCCCAGCCACCCAGCGG |
| | | ACAGAGAAGGGGAGGAUCCGGUGGUGGCGGCAGCGGCGGCAAGAAG |
| | | CAGUACAUCAAGGCCAACAGCAAGUUCAUCGGCAUCACCGAGCUGA |
| | | AGAAGCUGGGAGGGGGCAAACGGGGAGGCGGCAAAAAGAUGACCAA |
| | | CAGCGUGGACGACGCCCUGAUCAACAGCACCAAGAUCUACAGCUAC |
| | | UUCCCCAGCGUGAUCAGCAAAGUGAACCAGGGCGCUCAGGGCAAGA |
| | | AACUGGGCUCUAGCGGAGGGGGAGGCUCUCCUGGCGGGGGAUCUAG |
| | | CAUCGUGGGAAUUGUGGCAGGACUGGCAGUGCUGGCCGUGGUGGUG |
| | | AUCGGAGCCGUGGUGGCUACCGUGAUGUGCAGACGGAAGUCCAGCG |
| | | GAGGCAAGGGCGGCAGCUACAGCCAGGCCGCCAGCUCUGAUAGCGC |
| | | CCAGGGCAGCGACGUGUCACUGACAGCCUAGUAACUCGAGCUGGUA |
| | | CUGCAUGCACGCAAUGCUAGCUGCCCCUUUCCCGUCCUGGGUACCC |
| | | CGAGUCUCCCCCGACCUCGGGUCCCAGGUAUGCUCCCACCUCCACC |
| | | UGCCCCACUCACCACCUCUGCUAGUUCCAGACACCUCCCAAGCACG |
| | | CAGCAAUGCAGCUCAAAACGCUUAGCCUAGCCACACCCCCACGGGA |
| | | AACAGCAGUGAUUAACCUUUAGCAAUAAACGAAAGUUUAACUAAGC |
| | | UAUACUAACCCCAGGGUUGGUCAAUUUCGUGCCAGCCACACCGAGA |
| | | CCUGGUCCAGAGUCGCUAGCCGCGUCGCUAAAAAAAAAAAAAAAAA |
| | | AAAAAAAAAAAAAGCAUAUGACUAAAAAAAAAAAAAAAAAAAAAAA |
| | | AAAAAAAAAAAAAAAAAAAAAAAAAAAAAAAAAAAAAAAAAAAAAA |
| | | A |

Sec

| | | |
|---|---|---|
| 29 | sec | MRVMAPRTLILLLSGALALTETWAGS |
| | (amino acid) | |
| 30 | sec (CDS) | AUGAGAGUGAUGGCCCCCAGAACCCUGAUCCUGCUGCUGUCUGGCG |
| | | CCCUGGCCCUGACAGAGACAUGGGCCGGAAGC |

MITD

| | | |
|---|---|---|
| 31 | MITD | IVGIVAGLAVLAVVVIGAVVATVMCRRKSSGGKGGSYSQAASSDSA |
| | (amino acid) | QGSDVSLTA |
| 32 | MITD (CDS) | AUCGUGGGAAUUGUGGCAGGACUGGCAGUGCUGGCCGUGGUGGUGA |
| | | UCGGAGCCGUGGUGGCUACCGUGAUGUGCAGACGGAAGUCCAGCGG |
| | | AGGCAAGGGCGGCAGCUACAGCCAGGCCGCCAGCUCUGAUAGCGCC |
| | | CAGGGCAGCGACGUGUCACUGACAGCC |

P2P16 epitope

| | | |
|---|---|---|
| 33 | P2P16 | KKQYIKANSKFIGITELKKLGGGKRGGGKKMTNSVDDALINSTKIY |
| | (amino acid) | SYFPSVISKVNQGAQGKKL |
| 34 | P2P16 (CDS) | AAGAAGCAGUACAUCAAGGCCAACAGCAAGUUCAUCGGCAUCACCG |
| | | AGCUGAAGAAGCUGGGAGGGGGCAAACGGGGAGGCGGCAAAAAGAU |
| | | GACCAACAGCGUGGACGACGCCCUGAUCAACAGCACCAAGAUCUAC |
| | | AGCUACUUCCCCAGCGUGAUCAGCAAAGUGAACCAGGGCGCUCAGG |
| | | GCAAGAAACUG |

5′-UTR (hAg-Kozak)

| | | |
|---|---|---|
| 35 | 5′-UTR | AACUAGUAUUCUUCUGGUCCCCACAGACUCAGAGAGAACCCGCCAC |
| | | C |

3′-UTR (FI element)

| | | |
|---|---|---|
| 36 | 3′-UTR | CUGGUACUGCAUGCACGCAAUGCUAGCUGCCCCUUUCCCGUCCUGG |
| | | GUACCCCGAGUCUCCCCCGACCUCGGGUCCCAGGUAUGCUCCCACC |
| | | UCCACCUGCCCCACUCACCACCUCUGCUAGUUCCAGACACCUCCCA |
| | | AGCACGCAGCAAUGCAGCUCAAAACGCUUAGCCUAGCCACACCCCC |
| | | ACGGGAAACAGCAGUGAUUAACCUUUAGCAAUAAACGAAAGUUUAA |
| | | CUAAGCUAUACUAACCCCAGGGUUGGUCAAUUUCGUGCCAGCCACA |
| | | CC |

A30L70

| | | |
|---|---|---|
| 37 | A30L70 | AAAAAAAAAAAAAAAAAAAAAAAAAAAAAAGCAUAUGACUAAAAAA |
| | | AAAAAAAAAAAAAAAAAAAAAAAAAAAAAAAAAAAAAAAAAAAAAA |
| | | AAAAAAAAAAAAAAAAAA |

### DETAILED DESCRIPTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H.

KoNbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

The term “about” means approximately or nearly, and in the context of a numerical value or range set forth herein in one embodiment means ±20%, ±10%, ±5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context.

Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of”.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

### Definitions

In the following, definitions will be provided which apply to all aspects of the present disclosure. The following terms have the following meanings unless otherwise indicated. Any undefined terms have their art recognized meanings.

Terms such as “reduce”, “decrease”, “inhibit” or “impair” as used herein relate to an overall reduction or the ability to cause an overall reduction, preferably of at least 5%, at least 10%, at least 20%, at least 50%, at least 75% or even more, in the level. These terms include a complete or essentially complete inhibition, i.e., a reduction to zero or essentially to zero. Terms such as “increase”, “enhance” or “exceed” preferably relate to an increase or enhancement by at least 10%, at least 20%, at least 30%, at least 40%, at least 50%, at least 80%, at least 100%, at least 200%, at least 500%, or even more.

“Physiological pH” as used herein refers to a pH of about 7.5.

The term “ionic strength” refers to the mathematical relationship between the number of different kinds of ionic species in a particular solution and their respective charges. Thus, ionic strength I is represented mathematically by the formula

$$I = \frac{1}{2} \cdot {\sum }_{i} {z}_{i}^{2} \cdot {c}_{i}$$

in which c is the molar concentration of a particular ionic species and z the absolute value of its charge. The sum Σ is taken over all the different kinds of ions (i) in solution.

According to the disclosure, the term “ionic strength” in one embodiment relates to the presence of monovalent ions. Regarding the presence of divalent ions, in particular divalent cations, their concentration or effective concentration (presence of free ions) due to the presence of chelating agents is in one embodiment sufficiently low so as to prevent degradation of the RNA. In one embodiment, the concentration or effective concentration of divalent ions is below the catalytic level for hydrolysis of the phosphodiester bonds between RNA nucleotides. In one embodiment, the concentration of free divalent ions is 20 μM or less.

In one embodiment, there are no or essentially no free divalent ions.

The term “freezing” relates to the solidification of a liquid, usually with the removal of heat. The term “lyophilizing” or “lyophilization” refers to the freeze-drying of a substance by freezing it and then reducing the surrounding pressure to allow the frozen medium in the substance to sublimate directly from the solid phase to the gas phase.

The term “spray-drying” refers to spray-drying a substance by mixing (heated) gas with a fluid that is atomized (sprayed) within a vessel (spray dryer), where the solvent from the formed droplets evaporates, leading to a dry powder.

The term “cryoprotectant” relates to a substance that is added to a formulation in order to protect the active ingredients during the freezing stages.

The term “lyoprotectant” relates to a substance that is added to a formulation in order to protect the active ingredients during the drying stages.

The term “reconstitute” relates to adding a solvent such as water to a dried product to return it to a liquid state such as its original liquid state.

“Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

The term “recombinant” in the context of the present disclosure means “made through genetic engineering”. In one embodiment, a “recombinant object” in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

In the context of the present disclosure, the term “particle” relates to a structured entity formed by molecules or molecule complexes. In one embodiment, the term “particle” relates to a micro- or nano-sized structure, such as a micro- or nano-sized compact structure.

In the context of the present disclosure, the term “RNA lipoplex particle” relates to a particle that contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, a RNA lipoplex particle is a nanoparticle.

In particulate formulation, it is possible that each RNA species (e.g. RNA encoding the different vaccine antigens) is separately formulated as an individual particulate formulation. In that case, each individual particulate formulation will comprise one RNA species. The individual particulate formulations may be present as separate entities, e.g. in separate containers. Such formulations are obtainable by providing each RNA species separately (typically each in the form of an RNA-containing solution) together with a particle-forming agent, thereby allowing the formation of particles. Respective particles will contain exclusively the specific RNA species that is being provided when the particles are formed (individual particulate formulations). In one embodiment, a composition such as a pharmaceutical composition comprises more than one individual particle formulation. Respective pharmaceutical compositions are referred to as mixed particulate formulations. Mixed particulate formulations according to the invention are obtainable by forming, separately, individual particulate formulations, as described above, followed by a step of mixing of the individual particulate formulations. By the step of mixing, a formulation comprising a mixed population of RNA-containing particles is obtainable (for illustration: e.g. a first population of particles may contain RNA encoding a vaccine antigen, and a second formulation of particles may contain RNA encoding a different vaccine antigen).

Individual particulate populations may be together in one container, comprising a mixed population of individual particulate formulations. Alternatively, it is possible that different RNA species of the pharmaceutical composition (e.g. RNA encoding a vaccine antigen and RNA encoding a different vaccine antigen) are formulated together as a combined particulate formulation. Such formulations are obtainable by providing a combined formulation (typically combined solution) of different RNA species together with a particle-forming agent, thereby allowing the formation of particles. As opposed to a mixed particulate formulation, a combined particulate formulation will typically comprise particles which comprise more than one RNA species. In a combined particulate composition different RNA species are typically present together in a single particle.

As used in the present disclosure, “nanoparticle” refers to a particle comprising RNA and at least one cationic lipid and having an average diameter suitable for intravenous administration.

The term “average diameter” refers to the mean hydrodynamic diameter of particles as measured by dynamic light scattering (DLS) with data analysis using the so-called cumulant algorithm, which provides as results the so-called Z_{average} with the dimension of a length, and the polydispersity index (PI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

The term “polydispersity index” is used herein as a measure of the size distribution of an ensemble of particles, e.g., nanoparticles. The polydispersity index is calculated based on dynamic light scattering measurements by the so-called cumulant analysis.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion.

Using the ethanol injection technique, such colloidal liposome dispersion is, in one embodiment, formed as follows: an ethanol solution comprising lipids, such as cationic lipids like DOTMA and additional lipids, is injected into an aqueous solution under stirring. In one embodiment, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

As used herein, an “instructional material” or “instructions” includes a publication, a recording, a diagram, or any other medium of expression which can be used to communicate the usefulness of the compositions and methods of the invention. The instructional material of the kit of the invention may, for example, be affixed to a container which contains the compositions of the invention or be shipped together with a container which contains the compositions. Alternatively, the instructional material may be shipped separately from the container with the intention that the instructional material and the compositions be used cooperatively by the recipient.

As used herein, the term “vaccine” refers to a composition that induces an immune response upon inoculation into a subject. In some embodiments, the induced immune response provides therapeutic immunity.

Lung cancer, also known as lung carcinoma, is a malignant lung tumor characterized by uncontrolled cell growth in tissues of the lung. This growth can spread beyond the lung by the process of metastasis into nearby tissue or other parts of the body. Lung cancer is the third most frequent malignancy in women and the second most frequent malignancy in men and is the most common cause of cancer-related death in men and second most common in women after breast cancer. Lung cancers are carcinomas—malignancies that arise from epithelial cells. Lung carcinomas are categorized by the size and appearance of the malignant cells seen by a histopathologist under a microscope. For therapeutic purposes, two broad classes are distinguished: non-small-cell lung carcinoma (NSCLC) and small-cell lung carcinoma (SCLC). The three main subtypes of NSCLC are adenocarcinoma, squamous-cell carcinoma, and large-cell carcinoma. Rare subtypes include pulmonary enteric adenocarcinoma.

Nearly 40% of lung cancers are adenocarcinoma, which usually comes from peripheral lung tissue. Squamous-cell carcinoma causes about 30% of lung cancers. They typically occur close to large airways. A hollow cavity and associated cell death are commonly found at the center of the tumor. Nearly 9% of lung cancers are large-cell carcinoma. These are so named because the cancer cells are large, with excess cytoplasm, large nuclei, and conspicuous nucleoli.

The term “co-administered” or “co-administration” or the like as used herein refers to administration of two or more agents concurrently, simultaneously, or essentially at the same time, either as part of a single formulation or as multiple formulations that are administered by the same or different routes. “Essentially at the same time” as used herein means within about 1 minute, 5 minutes, 10 minutes, 15 minutes, 30 minutes, 1 hour, 2 hours, or 6 hours period of each other.

The disclosure describes nucleic acid sequences and amino acid sequences having a certain degree of identity to a given nucleic acid sequence or amino acid sequence, respectively (a reference sequence).

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical”, “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, −2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. One important property includes an immunogenic property, in particular when administered to a subject. In some embodiments, a nucleic acid sequence or amino acid sequence having a particular degree of identity to a given nucleic acid sequence or amino acid sequence is functionally equivalent to the given sequence.

### RNA

In the present disclosure, the term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered RNAs are considered analogs of naturally-occurring RNA.

In certain embodiments of the present disclosure, the RNA is messenger RNA (mRNA) that relates to a RNA transcript which encodes a peptide or protein. As established in the art, mRNA generally contains a 5′-untranslated region (5′-UTR), a peptide coding region and a 3′-untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

In one embodiment, RNA is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In one embodiment, the RNA may have modified nucleosides. In some embodiments, the RNA comprises a modified nucleoside in place of at least one (e.g., every) uridine.

The term “uracil,” as used herein, describes one of the nucleobases that can occur in the nucleic acid of RNA. The structure of uracil is:

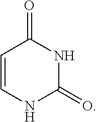

The term “uridine,” as used herein, describes one of the nucleosides that can occur in RNA.

The structure of uridine is:

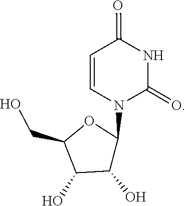 [SMILES: O=c1ccn([C@@H]2O[C@H](CO)[C@@H](O)[C@H]2O)c(=O)[nH]1]

UTP (uridine 5′-triphosphate) has the following structure:

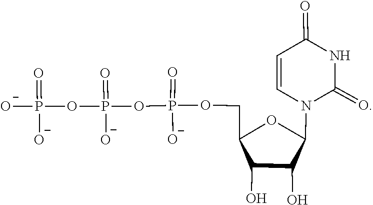

Pseudo-UTP (pseudouridine 5′-triphosphate) has the following structure:

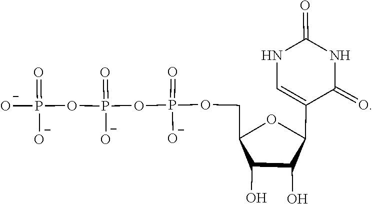

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1L4), which has the structure:

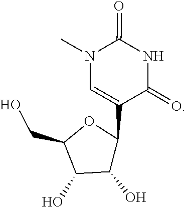 [SMILES: Cn1cc([C@@H]2O[C@H](CO)[C@@H](O)[C@H]2O)c(=O)[nH]c1=O]

N1-methyl-pseudo-UTP has the following structure:

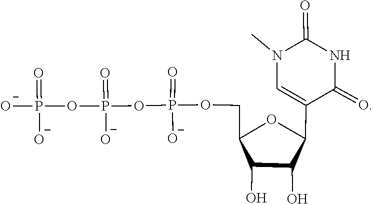

Another exemplary modified nucleoside is 5-methyl-uridine (m5U), which has the structure:

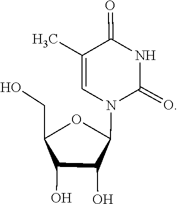

In some embodiments, one or more uridine in the RNA described herein is replaced by a modified nucleoside. In some embodiments, the modified nucleoside is a modified uridine.

In some embodiments, the modified uridine replacing uridine is pseudouridine (ψ), N1-methyl-pseudouridine (m1ψ), or 5-methyl-uridine (m5U).

In some embodiments, the modified nucleoside replacing one or more, e.g., all, uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s2U), 5-aminomethyl-2-thio-uridine (nm⁵s2U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s2U), 5-propynyl-uridine, 1-propynyl-pseudouridine, 5-taurinomethyl-uridine (ρm⁵U), 1-taurinomethyl-pseudouridine, 5-taurinomethyl-2-thio-uridine(ρm⁵s²U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³L), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Urn), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In some embodiments, at least one RNA comprises a modified nucleoside in place of at least one uridine. In some embodiments, at least one RNA comprises a modified nucleoside in place of each uridine. In some embodiments, each RNA comprises a modified nucleoside in place of at least one uridine. In some embodiments, each RNA comprises a modified nucleoside in place of each uridine.

In some embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methyl-uridine (m5U). In some embodiments, at least one RNA may comprise more than one type of modified nucleoside, and the modified nucleosides are independently selected from pseudouridine (ψ), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ)), N1-methyl-pseudouridine (m1ψ), and 5-methyl-uridine (m5U).

In one embodiment, the RNA comprises other modified nucleosides or comprises further modified nucleosides, e.g., modified cytidine. For example, in one embodiment, in the RNA 5-methylcytidine is substituted partially or completely, preferably completely, for cytidine. In one embodiment, the RNA comprises 5-methylcytidine and one or more selected from pseudouridine (ψ), N1-methyl-pseudouridine (m1ψ), and 5-methyl-uridine (m5U). In one embodiment, the RNA comprises 5-methylcytidine and N1-methyl-pseudouridine (m1ψ). In some embodiments, the RNA comprises 5-methylcytidine in place of each cytidine and N1-methyl-pseudouridine (m1ψ) in place of each uridine.

In some embodiments, the RNA according to the present disclosure comprises a 5′-cap. In one embodiment, the RNA of the present disclosure does not have uncapped 5′-triphosphates. In one embodiment, the RNA may be modified by a 5′-cap analog. The term “5′-cap” refers to a structure found on the 5′-end of an mRNA molecule and generally consists of a guanosine nucleotide connected to the mRNA via a 5′- to 5′-triphosphate linkage. In one embodiment, this guanosine is methylated at the 7-position. Providing an RNA with a 5′-cap or 5′-cap analog may be achieved by in vitro transcription, in which the 5′-cap is co-transcriptionally expressed into the RNA strand, or may be attached to RNA post-transcriptionally using capping enzymes.

In some embodiments, the building block cap for RNA is m₂^{7,3′O}Gppp(m₁^{2′-O})ApG (also sometimes referred to as m₂^{73′O}G(5′)ppp(5′)m^{2′-O}ApG), which has the following structure:

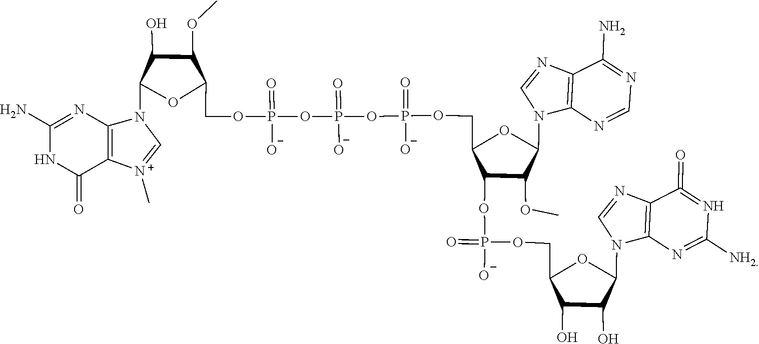

Below is an exemplary Cap1 RNA, which comprises RNA and m₂^{7,3′O}G(5′)ppp(5′)m^{2′-O}ApG:

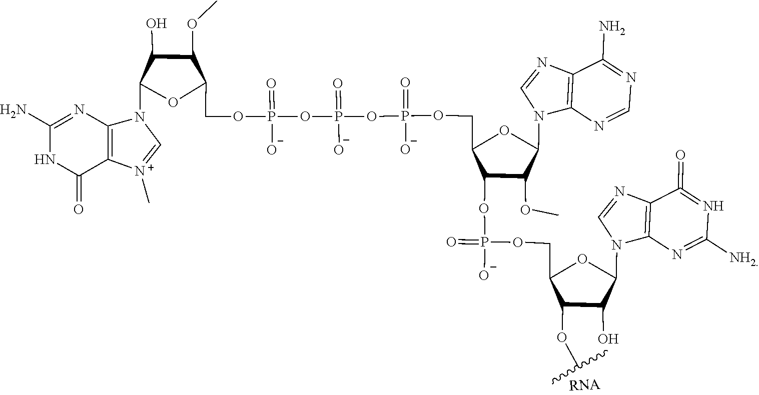

Below is another exemplary Cap1 RNA (no cap analog):

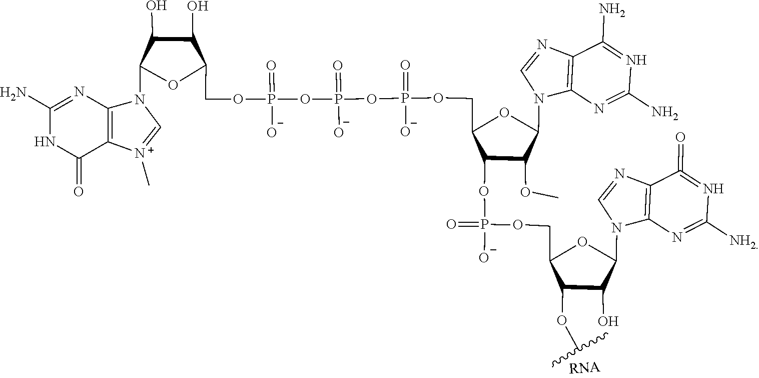

In some embodiments, the RNA is modified with “Cap0” structures using, in one embodiment, the cap analog anti-reverse cap (ARCA Cap (m₂^{7,3′O}G(5′)ppp(5′)G)) with the structure:

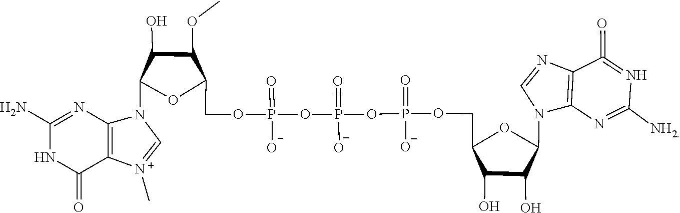

Below is an exemplary Cap0 RNA comprising RNA and m₂^{7,3′O}G(5′)ppp(5′)G:

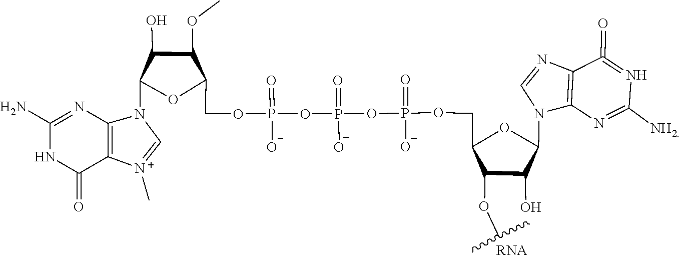

In some embodiments, the “Cap0” structures are generated using the cap analog Beta-S-ARCA (m₂^{7,2′O}G(5′)ppSp(5′)G) with the structure:

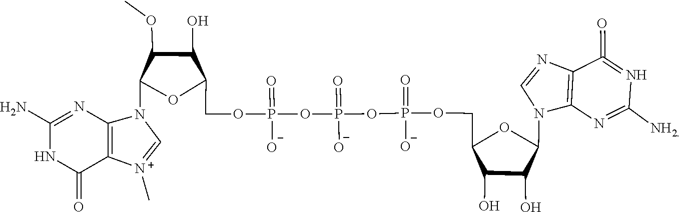

Below is an exemplary Cap0 RNA comprising Beta-S-ARCA (m₂^{7,2′O}G(5′)ppSp(5′)G) and RNA:

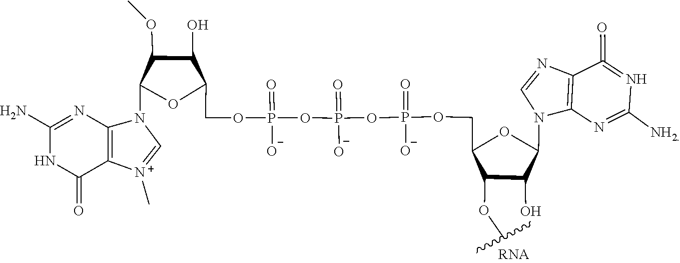

A particularly preferred Cap comprises the 5′-cap m₂^{7,2′O}G(5′)ppSp(5′)G. In some embodiments, at least one RNA described herein comprises the 5′-cap m₂^{7,2′O}G(5′)ppSp(5′)G.

In some embodiments, each RNA described herein comprises the 5′-cap m₂^{7,2′O}G(5′)ppSp(5′)G. The “D1” diastereomer of Beta-S-ARCA or “Beta-S-ARCA(D1)” is the diastereomer of Beta-S-ARCA which elutes first on an HPLC column compared to the D2 diastereomer of Beta-S-ARCA (Beta-S-ARCA(D2)) and thus exhibits a shorter retention time (cf., WO 2011/015347, herein incorporated by reference). A particularly preferred cap is Beta-S-ARCA(D1) (m₂^{7,2′-O}GppSpG). In some embodiments, RNA according to the present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′-end, upstream of the start codon of a protein-encoding region. A 5′-UTR is downstream of the 5′-cap (if present), e.g., directly adjacent to the 5′-cap. A 3′-UTR, if present, is located at the 3′-end, downstream of the termination codon of a protein-encoding region, but the term “3′-UTR” does preferably not include the poly-A sequence. Thus, the 3′-UTR is upstream of the poly-A sequence (if present), e.g., directly adjacent to the poly-A sequence.

A particularly preferred 5′-UTR comprises the nucleotide sequence of SEQ ID NO: 35. A particularly preferred 3′-UTR comprises the nucleotide sequence of SEQ ID NO: 36.

In some embodiments, at least one RNA comprises a 5′-UTR comprising the nucleotide sequence of SEQ ID NO: 35, or a nucleotide sequence having at least 99%, 98%, 97%, 96%, 95%, 90%, 85%, or 80% identity to the nucleotide sequence of SEQ ID NO: 35. In some embodiments, each RNA comprises a 5′-UTR comprising the nucleotide sequence of SEQ ID NO: 35, or a nucleotide sequence having at least 99%, 98%, 97%, 96%, 95%, 90%, 85%, or 80% identity to the nucleotide sequence of SEQ ID NO: 35.

In some embodiments, at least one RNA comprises a 3′-UTR comprising the nucleotide sequence of SEQ ID NO: 36, or a nucleotide sequence having at least 99%, 98%, 97%, 96%, 95%, 90%, 85%, or 80% identity to the nucleotide sequence of SEQ ID NO: 36. In some embodiments, each RNA comprises a 3′-UTR comprising the nucleotide sequence of SEQ ID NO: 36, or a nucleotide sequence having at least 99%, 98%, 97%, 96%, 95%, 90%, 85%, or 80% identity to the nucleotide sequence of SEQ ID NO: 36.

In some embodiments, the RNA according to the present disclosure comprises a 3′-poly(A) sequence.

As used herein, the term “poly-A tail” or “poly-A sequence” refers to an uninterrupted or interrupted sequence of adenylate residues which is typically located at the 3′-end of an RNA molecule. Poly-A tails or poly-A sequences are known to those of skill in the art and may follow the 3′-UTR in the RNAs described herein. An uninterrupted poly-A tail is characterized by consecutive adenylate residues. In nature, an uninterrupted poly-A tail is typical. RNAs disclosed herein can have a poly-A tail attached to the free 3-end of the RNA by a template-independent RNA polymerase after transcription or a poly-A tail encoded by DNA and transcribed by a template-dependent RNA polymerase.

It has been demonstrated that a poly-A tail of about 120 A nucleotides has a beneficial influence on the levels of RNA in transfected eukaryotic cells, as well as on the levels of protein that is translated from an open reading frame that is present upstream (5′) of the poly-A tail (Holtkamp et al., 2006, Blood, vol. 108, pp. 4009-4017).

The poly-A tail may be of any length. In some embodiments, a poly-Atail comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly-A tail, typically at least 75%, at least 80%, at least 85%, at least 90%, at least 95%, at least 96%, at least 97%, at least 98%, or at least 99% by number of nucleotides in the poly-A tail are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly-A tail, i.e., 100% by number of nucleotides in the poly-A tail, are A nucleotides. The term “A nucleotide” or “A” refers to adenylate.

In some embodiments, a poly-A tail is attached during RNA transcription, e.g., during preparation of in vitro transcribed RNA, based on a DNA template comprising repeated dT nucleotides (deoxythymidylate) in the strand complementary to the coding strand. The DNA sequence encoding a poly-A tail (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT). Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present invention. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly-A tail contained in an RNA molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly-A tail at its 3′-end, i.e., the poly-A tail is not masked or followed at its 3′-end by a nucleotide other than A.

In some embodiments, a poly-A tail comprises the sequence of SEQ ID NO: 37.

In some embodiments, at least one RNA comprises a poly-A tail. In some embodiments, each RNA comprises a poly-A tail. In some embodiments, the poly-A tail may comprise at least 20, at least 30, at least 40, at least 80, or at least 100 and up to 500, up to 400, up to 300, up to 200, or up to 150 nucleotides. In some embodiments, the poly-A tail may essentially consist of at least 20, at least 30, at least 40, at least 80, or at least 100 and up to 500, up to 400, up to 300, up to 200, or up to 150 nucleotides. In some embodiments, the poly-A tail may consist of at least 20, at least 30, at least 40, at least 80, or at least 100 and up to 500, up to 400, up to 300, up to 200, or up to 150 nucleotides. In some embodiments, the poly-A tail may comprise the poly-A tail shown in SEQ ID NO: 37. In some embodiments, the poly-A tail comprises at least 100 nucleotides. In some embodiments, the poly-A tail comprises about 150 nucleotides. In some embodiments, the poly-A tail comprises about 120 nucleotides.

In some embodiments, at least one RNA comprises a poly-A tail comprising the nucleotide sequence of SEQ ID NO: 37, or a nucleotide sequence having at least 99%, 98%, 97%, 96%, 95%, 90%, 85%, or 80% identity to the nucleotide sequence of SEQ ID NO: 37. In some embodiments, each RNA comprises a poly-A tail comprising the nucleotide sequence of SEQ ID NO: 37, or a nucleotide sequence having at least 99%, 98%, 97%, 96%, 95%, 90%, 85%, or 80% identity to the nucleotide sequence of SEQ ID NO: 37.

In the context of the present disclosure, the term “transcription” relates to a process, wherein the genetic code in a DNA sequence is transcribed into RNA. Subsequently, the RNA may be translated into peptide or protein.

According to the present invention, the term “transcription” comprises “in vitro transcription”, wherein the term “in vitro transcription” relates to a process wherein RNA, in particular mRNA, is in vitro synthesized in a cell-free system, preferably using appropriate cell extracts.

Preferably, cloning vectors are applied for the generation of transcripts. These cloning vectors are generally designated as transcription vectors and are according to the present invention encompassed by the term “vector”. According to the present invention, the RNA used in the present invention preferably is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

With respect to RNA, the term “expression” or “translation” relates to the process in the ribosomes of a cell by which a strand of mRNA directs the assembly of a sequence of amino acids to make a peptide or protein.

In one embodiment, after administration of the RNA described herein, e.g., formulated as RNA lipoplex particles, at least a portion of the RNA is delivered to a target cell. In one embodiment, at least a portion of the RNA is delivered to the cytosol of the target cell. In one embodiment, the RNA is translated by the target cell to produce the peptide or protein it enodes. In one embodiment, the target cell is a spleen cell. In one embodiment, the target cell is an antigen presenting cell such as a professional antigen presenting cell in the spleen. In one embodiment, the target cell is a dendritic cell or macrophage. RNA lipoplex particles described herein may be used for delivering RNA to such target cell. Accordingly, the present disclosure also relates to a method for delivering RNA to a target cell in a subject comprising the administration of the RNA lipoplex particles described herein to the subject. In one embodiment, the RNA is delivered to the cytosol of the target cell. In one embodiment, the RNA is translated by the target cell to produce the peptide or protein encoded by the RNA.

According to the disclosure, the term “RNA encodes” means that the RNA, if present in the appropriate environment, such as within cells of a target tissue, can direct the assembly of amino acids to produce the peptide or protein it encodes during the process of translation. In one embodiment, RNA is able to interact with the cellular translation machinery allowing translation of the peptide or protein. A cell may produce the encoded peptide or protein intracellularly (e.g., in the cytoplasm and/or in the nucleus), may secrete the encoded peptide or protein, or may produce it on the surface.

According to the disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” refers to large peptides, in particular peptides having at least about 151 amino acids, but the terms “peptide” and “protein” are used herein usually as synonyms.

The term “antigen” relates to an agent comprising an epitope against which an immune response can be generated. The term “antigen” includes, in particular, proteins and peptides. In one embodiment, an antigen is presented by cells of the immune system such as antigen presenting cells like dendritic cells or macrophages. An antigen or a processing product thereof such as a T-cell epitope is in one embodiment bound by a T- or B-cell receptor, or by an immunoglobulin molecule such as an antibody. Accordingly, an antigen or a processing product thereof may react specifically with antibodies or T lymphocytes (T cells). In one embodiment, an antigen is a disease-associated antigen, such as a tumor antigen and an epitope is derived from such antigen.

The term “disease-associated antigen” is used in its broadest sense to refer to any antigen associated with a disease. A disease-associated antigen is a molecule which contains epitopes that will stimulate a host's immune system to make a cellular antigen-specific immune response and/or a humoral antibody response against the disease. The disease-associated antigen or an epitope thereof may therefore be used for therapeutic purposes. Disease-associated antigens may be associated with cancer, typically tumors.

The term “tumor antigen” refers to a constituent of cancer cells which may be derived from the cytoplasm, the cell surface and the cell nucleus. In particular, it refers to those antigens which are produced intracellularly or as surface antigens on tumor cells.

A tumor antigen disclosed herein may be CLDN6 (SEQID NO: 1), KK-LC-1 (SEQ ID NO: 5), MAGE-A3 (SEQ ID NO: 9), MAGE-A4 (SEQ ID NO: 13), PRAME (SEQ ID NO: 17), MAGE-C1 (SEQ ID NO: 21) or NY-ESO-1 (SEQ ID NO: 25).

The term “epitope” refers to a part or fragment a molecule such as an antigen that is recognized by the immune system. For example, the epitope may be recognized by T cells, B cells or antibodies. An epitope of an antigen may include a continuous or discontinuous portion of the antigen and may be between about 5 and about 100 amino acids in length. In one embodiment, an epitope is between about 10 and about 25 amino acids in length. The term “epitope” includes T-cell epitopes.

The term “T-cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T-cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

In certain embodiments of the present disclosure, the RNA encodes at least one epitope. In certain embodiments, the epitope is derived from a tumor antigen as described herein.

In some embodiment, the amino acid sequence comprising a tumor antigen, an immunogenic variant thereof, or an immunogenic fragment of the tumor antigen or the immunogenic variant thereof described herein is encoded by a coding sequence which is codon-optimized and/or the G/C content of which is increased compared to wild type coding sequence. This also includes embodiments, wherein one or more sequence regions of the coding sequence are codon-optimized and/or increased in the G/C content compared to the corresponding sequence regions of the wild type coding sequence. In one embodiment, the codon-optimization and/or the increase in the G/C content preferably does not change the sequence of the encoded amino acid sequence.

The term “codon-optimized” refers to the alteration of codons in the coding region of a nucleic acid molecule to reflect the typical codon usage of a host organism without preferably altering the amino acid sequence encoded by the nucleic acid molecule. Within the context of the present invention, coding regions are preferably codon-optimized for optimal expression in a subject to be treated using the RNA molecules described herein. Codon-optimization is based on the finding that the translation efficiency is also determined by a different frequency in the occurrence of tRNAs in cells. Thus, the sequence of RNA may be modified such that codons for which frequently occurring tRNAs are available are inserted in place of “rare codons”.

In some embodiments of the invention, the guanosine/cytosine (G/C) content of the coding region of the RNA described herein is increased compared to the G/C content of the corresponding coding sequence of the wild type RNA, wherein the amino acid sequence encoded by the RNA is preferably not modified compared to the amino acid sequence encoded by the wild type RNA. This modification of the RNA sequence is based on the fact that the sequence of any RNA region to be translated is important for efficient translation of that mRNA. Sequences having an increased G (guanosine)/C (cytosine) content are more stable than sequences having an increased A (adenosine)/U (uracil) content. In respect to the fact that several codons code for one and the same amino acid (so-called degeneration of the genetic code), the most favourable codons for the stability can be determined (so-called alternative codon usage). Depending on the amino acid to be encoded by the RNA, there are various possibilities for modification of the RNA sequence, compared to its wild type sequence. In particular, codons which contain A and/or U nucleotides can be modified by substituting these codons by other codons, which code for the same amino acids but contain no A and/or U or contain a lower content of A and/or U nucleotides.

In various embodiments, the G/C content of the coding region of the RNA described herein is increased by at least 10%, at least 20%, at least 30%, at least 40%, at least 50%, at least 55%, or even more compared to the G/C content of the coding region of the wild type RNA.

### Administered RNAs

In some embodiments, compositions or medical preparations described herein comprise RNA encoding a claudin 6 (CLDN6) vaccine antigen, RNA encoding a Kita-kyushu lung cancer antigen 1 (KK-LC-1) vaccine antigen, RNA encoding a Melanoma antigen A3 (MAGE-A3) vaccine antigen, RNA encoding a Melanoma antigen 4 (MAGE-A4) vaccine antigen, RNA encoding a Preferentially Expressed Antigen In Melanoma (PRAME) vaccine antigen, and one or both of RNA encoding a Melanoma antigen C1 (MAGE-C1) vaccine antigen and RNA encoding a New York esophageal squamous cell carcinoma-1 (NY-ESO-1) vaccine antigen. In some embodiments, compositions or medical preparations described herein comprise RNA encoding a claudin 6 (CLDN6) vaccine antigen, RNA encoding a Kita-kyushu lung cancer antigen 1 (KK-LC-1) vaccine antigen, RNA encoding a Melanoma antigen A3 (MAGE-A3) vaccine antigen, RNA encoding a Melanoma antigen 4 (MAGE-A4) vaccine antigen, RNA encoding a Preferentially Expressed Antigen In Melanoma (PRAME) vaccine antigen, and RNA encoding a Melanoma antigen C1 (MAGE-C1) vaccine antigen. Likewise, methods described herein comprise administration of RNA encoding a claudin 6 (CLDN6) vaccine antigen, RNA encoding a Kita-kyushu lung cancer antigen 1 (KK-LC-1) vaccine antigen, RNA encoding a Melanoma antigen A3 (MAGE-A3) vaccine antigen, RNA encoding a Melanoma antigen 4 (MAGE-A4) vaccine antigen, RNA encoding a Preferentially Expressed Antigen In Melanoma (PRAME) vaccine antigen, and one or both of RNA encoding a Melanoma antigen C1 (MAGE-C1) vaccine antigen and RNA encoding a New York esophageal squamous cell carcinoma-1 (NY-ESO-1) vaccine antigen. In some embodiments, methods described herein comprise administration of RNA encoding a claudin 6 (CLDN6) vaccine antigen, RNA encoding a Kita-kyushu lung cancer antigen 1 (KK-LC-1) vaccine antigen, RNA encoding a Melanoma antigen A3 (MAGE-A3) vaccine antigen, RNA encoding a Melanoma antigen 4 (MAGE-A4) vaccine antigen, RNA encoding a Preferentially Expressed Antigen In Melanoma (PRAME) vaccine antigen, and RNA encoding a Melanoma antigen C1 (MAGE-C1) vaccine antigen.

### Molecular Structure and Function of CLDN6 Vaccine Antigen

The human claudin 6 gene (CLDN6) is localized on chromosome 16 and contains two isoforms which encode a protein of 220 amino acids. CLDN6 is highly conserved among species, and belongs to the group of claudins which consists of at least 27 members. In general, claudins, including CLDN6, are important for epithelial barrier regulation and belong to the group of tight junction molecules. CLDN6 contains four transmembrane domains, two extracellular loops, intracellular N- and C-termini, and a PDZ-binding domain, and has been shown to play a role in maintaining permeability barriers and trans-epithelial resistance in epidermal cells. Additionally, CLDN6 appears to be required for normal blastocyst formation. In one embodiment, CLDN6 has the amino acid sequence according to SEQ ID NO: 1.

A claudin 6 (CLDN6) vaccine antigen comprises an amino acid sequence comprising CLDN6, an immunogenic variant thereof, or an immunogenic fragment of the CLDN6 or the immunogenic variant thereof, and may have an amino acid sequence comprising the amino acid sequence of SEQ ID NO: 1 or 2, or an amino acid sequence having at least 99%, 98%, 97%, 96%, 95%, 90%, 85%, or 80% identity to the amino acid sequence of SEQ ID NO: 1 or 2. RNA encoding a CLDN6 vaccine antigen (i) may comprise the nucleotide sequence of SEQ ID NO: 3 or 4, or a nucleotide sequence having at least 99%, 98%, 97%, 96%, 95%, 90%, 85%, or 80% identity to the nucleotide sequence of SEQ ID NO: 3 or 4; and/or (ii) may encode an amino acid sequence comprising the amino acid sequence of SEQ ID NO: 1 or 2, or an amino acid sequence having at least 99%, 98%, 97%, 96%, 95%, 90%, 85%, or 80% identity to the amino acid sequence of SEQ ID NO: 1 or 2.

### Molecular Structure and Function of KK-LC-1 Vaccine Antigen

Kita-kyushu lung cancer antigen 1 (KK-LC-1), also cancer/testis antigen 83, CT83, CXorf61, is a protein and tumor antigen from the group of cancer/testis antigens. KK-LC-1 has a length of 113 amino acids. KK-LC-1 is rarely found as a tumor antigen in healthy cells (except in immune privileged spermatocytes), but is often expressed in various tumors, e.g. non-small cell lung cancer. In one embodiment, KK-LC-1 has the amino acid sequence according to SEQ ID NO: 5.

A Kita-kyushu lung cancer antigen 1 (KK-LC-1) vaccine antigen comprises an amino acid sequence comprising KK-LC-1, an immunogenic variant thereof, or an immunogenic fragment of the KK-LC-1 or the immunogenic variant thereof, and may have an amino acid sequence comprising the amino acid sequence of SEQ ID NO: 5 or 6, or an amino acid sequence having at least 99%, 98%, 97%, 96%, 95%, 90%, 85%, or 80% identity to the amino acid sequence of SEQ ID NO: 5 or 6. RNA encoding a KK-LC-1 vaccine antigen (i) may comprise the nucleotide sequence of SEQ ID NO: 7 or 8, or a nucleotide sequence having at least 99%, 98%, 97%, 96%, 95%, 90%, 85%, or 80% identity to the nucleotide sequence of SEQ ID NO: 7 or 8; and/or (ii) may encode an amino acid sequence comprising the amino acid sequence of SEQ ID NO: 5 or 6, or an amino acid sequence having at least 99%, 98%, 97%, 96%, 95%, 90%, 85%, or 80% identity to the amino acid sequence of SEQ ID NO: 5 or 6.

### Molecular Structure and Function of MAGE-A3 Vaccine Antigen

The human Melanoma antigen A3 (MAGE-A3) gene is a member of the melanoma-associated antigen gene family. The members of this family encode proteins with 50 to 80% sequence identity to each other. The MAGEA genes are clustered at chromosomal location Xq28. They have been implicated in some hereditary disorders, such as dyskeratosis congenita. The normal function of MAGE-A3 in healthy cells is unknown. In one embodiment, MAGE-A3 has the amino acid sequence according to SEQ ID NO: 9.

A Melanoma antigen A3 (MAGE-A3) vaccine antigen comprises an amino acid sequence comprising MAGE-A3, an immunogenic variant thereof, or an immunogenic fragment of the MAGE-A3 or the immunogenic variant thereof, and may have an amino acid sequence comprising the amino acid sequence of SEQ ID NO: 9 or 10, or an amino acid sequence having at least 99%, 98%, 97%, 96%, 95%, 90%, 85%, or 80% identity to the amino acid sequence of SEQID NO: 9 or 10. RNA encoding a MAGE-A3 vaccine antigen (i) may comprise the nucleotide sequence of SEQ ID NO: 11 or 12, or a nucleotide sequence having at least 99%, 98%, 97%, 96%, 95%, 90%, 85%, or 80% identity to the nucleotide sequence of SEQ ID NO: 11 or 12; and/or (ii) may encode an amino acid sequence comprising the amino acid sequence of SEQ ID NO: 9 or 10, or an amino acid sequence having at least 99%, 98%, 97%, 96%, 95%, 90%, 85%, or 80% identity to the amino acid sequence of SEQ ID NO: 9 or 10.

### Molecular Structure and Function of MAGE-A4 Vaccine Antigen

The human Melanoma antigen 4 (MAGE-A4) gene is a member of the MAGEA gene family. The members of this family encode proteins with 50 to 80% sequence identity to each other. The MAGEA genes are clustered at chromosomal location Xq28. They have been implicated in some hereditary disorders, such as dyskeratosis congenita. In one embodiment, MAGE-A4 has the amino acid sequence according to SEQ ID NO: 13.

A Melanoma antigen 4 (MAGE-A4) vaccine antigen comprises an amino acid sequence comprising MAGE-A4, an immunogenic variant thereof, or an immunogenic fragment of the MAGE-A4 or the immunogenic variant thereof, and may have an amino acid sequence comprising the amino acid sequence of SEQ ID NO: 13 or 14, or an amino acid sequence having at least 99%, 98%, 97%, 96%, 95%, 90%, 85%, or 80% identity to the amino acid sequence of SEQ ID NO: 13 or 14. RNA encoding a MAGE-A4 vaccine antigen (i) may comprise the nucleotide sequence of SEQ ID NO: 15 or 16, or a nucleotide sequence having at least 99%, 98%, 97%, 96%, 95%, 90%, 85%, or 80% identity to the nucleotide sequence of SEQ ID NO: 15 or 16; and/or (ii) may encode an amino acid sequence comprising the amino acid sequence of SEQ ID NO: 13 or 14, or an amino acid sequence having at least 99%, 98%, 97%, 96%, 95%, 90%, 85%, or 80% identity to the amino acid sequence of SEQ ID NO: 13 or 14.

### Molecular Structure and Function of PRAME Vaccine Antigen

The human preferentially expressed in melanoma (PRAME) gene is localized on chromosome 22 and contains eight isoforms out of which seven encode for an identical protein of 509 amino acids, while the eighth isoform lacks the first 16 amino acids. Localization studies using FLAG- or GFP-tagged PRAME suggest a nuclear localization of the protein. Furthermore, PRAME plays a critical role in apoptosis and cell proliferation. Further functional studies revealed that PRAME inhibits retinoic acid receptor signaling and thereby elicits its role in apoptosis and differentiation. PRAME belongs to a multigene family consisting of 32 PRAME-like genes and pseudogenes. The closest protein-coding relatives of PRAME exhibit 53% homology to the protein (using the blastp command of the blast software package). A detailed RT-qPCR-based analysis revealed a high expression of PRAME in testis, epididymis and uterus. In one embodiment, PRAME has the amino acid sequence according to SEQ ID NO: 17.

A Preferentially Expressed Antigen In Melanoma (PRAME) vaccine antigen comprises an amino acid sequence comprising PRAME, an immunogenic variant thereof, or an immunogenic fragment of the PRAME or the immunogenic variant thereof, and may have an amino acid sequence comprising the amino acid sequence of SEQ ID NO: 17 or 18, or an amino acid sequence having at least 99%, 98%, 97%, 96%, 95%, 90%, 85%, or 80% identity to the amino acid sequence of SEQ ID NO: 17 or 18. RNA encoding a PRAME vaccine antigen (i) may comprise the nucleotide sequence of SEQ ID NO: 19 or 20, or a nucleotide sequence having at least 99%, 98%, 97%, 96%, 95%, 90%, 85%, or 80% identity to the nucleotide sequence of SEQ ID NO: 19 or 20; and/or (ii) may encode an amino acid sequence comprising the amino acid sequence of SEQ ID NO: 17 or 18, or an amino acid sequence having at least 99%, 98%, 97%, 96%, 95%, 90%, 85%, or 80% identity to the amino acid sequence of SEQ ID NO: 17 or 18.

### Molecular Structure and Function of MAGE-C1 Vaccine Antigen

Melanoma antigen C1 (MAGE-C1), also cancer/testis antigen 7 (CT7), is a human tumor antigen from the group of cancer/testis antigens. MAGE-C1 has a length of 1,142 amino acids and a mass of 123,643 Da. It is phosphorylated on up to four serines, S63, S207, S382 and S1063. MAGE-C1 has anti-apoptotic properties and binds to NY-ESO-1. It does not occur in healthy cells (except in immune-privileged spermatocytes), but is often expressed in tumors, e.g. multiple myelomas. There it is formed by malignant plasma cells. In one embodiment, MAGE-C1 has the amino acid sequence according to SEQ ID NO: 21.

A Melanoma antigen C1 (MAGE-C1) vaccine antigen comprises an amino acid sequence comprising MAGE-C1, an immunogenic variant thereof, or an immunogenic fragment of the MAGE-C1 or the immunogenic variant thereof, and may have an amino acid sequence comprising the amino acid sequence of SEQ ID NO: 21 or 22, or an amino acid sequence having at least 99%, 98%, 97%, 96%, 95%, 90%, 85%, or 80% identity to the amino acid sequence of SEQ ID NO: 21 or 22. RNA encoding a MAGE-C1 vaccine antigen (i) may comprise the nucleotide sequence of SEQ ID NO: 23 or 24, or a nucleotide sequence having at least 99%, 98%, 97%, 96%, 95%, 90%, 85%, or 80% identity to the nucleotide sequence of SEQ ID NO: 23 or 24; and/or (ii) may encode an amino acid sequence comprising the amino acid sequence of SEQ ID NO: 21 or 22, or an amino acid sequence having at least 99%, 98%, 97%, 96%, 95%, 90%, 85%, or 80% identity to the amino acid sequence of SEQ ID NO: 21 or 22.

### Molecular Structure and Function of NY-ESO-1 Vaccine Antigen

New York esophageal squamous cell carcinoma-1 (NY-ESO-1), also cancer/testis antigen 1, LAGE2 or LAGE2B, is a protein that in humans is encoded by the CTAG1B gene. CTAG1B is located on the long arm of chromosome X (Xq28). The gene encodes a 180-amino acid polypeptide, expressed from 18 weeks during embryonic development until birth in human fetal testis. It is also strongly expressed in spermatogonia and in primary spermatocytes of adult testis, but not in post-meiotic cells or testicular somatic cells. NY-ESO-1 belongs to the family of Cancer Testis Antigens (CTA) that are expressed in a variety of malignant tumours at the mRNA and protein levels, but also restricted to testicular germ cells in normal adult tissues. In one embodiment, NY-ESO-1 has the amino acid sequence according to SEQ ID NO: 25.

A New York esophageal squamous cell carcinoma-1 (NY-ESO-1) vaccine antigen comprises an amino acid sequence comprising NY-ESO-1, an immunogenic variant thereof, or an immunogenic fragment of the NY-ESO-1 or the immunogenic variant thereof, and may have an amino acid sequence comprising the amino acid sequence of SEQ ID NO: 25 or 26, or an amino acid sequence having at least 99%, 98%, 97%, 96%, 95%, 90%, 85%, or 80% identity to the amino acid sequence of SEQ ID NO: 25 or 26. RNA encoding a NY-ESO-1 vaccine antigen (i) may comprise the nucleotide sequence of SEQ ID NO: 27 or 28, or a nucleotide sequence having at least 99%, 98%, 97%, 96%, 95%, 90%, 85%, or 80% identity to the nucleotide sequence of SEQ ID NO: 27 or 28; and/or (ii) may encode an amino acid sequence comprising the amino acid sequence of SEQ ID NO: 25 or 26, or an amino acid sequence having at least 99%, 98%, 97%, 96%, 95%, 90%, 85%, or 80% identity to the amino acid sequence of SEQ ID NO: 25 or 26.

Amino acid sequences derived from tetanus toxoid of Clostridium tetani may be employed to overcome self-tolerance mechanisms in order to efficiently mount an immune response to self-antigens by providing T-cell help during priming.

It is known that tetanus toxoid heavy chain includes epitopes that can bind promiscuously to MHC class II alleles and induce CD4⁺ memory T cells in almost all tetanus vaccinated individuals. In addition, the combination of tetanus toxoid (TT) helper epitopes with tumor-associated antigens is known to improve the immune stimulation compared to application of tumor-associated antigen alone by providing CD4⁺-mediated T-cell help during priming. To reduce the risk of stimulating CD8⁺ T cells with the tetanus sequences which might compete with the intended induction of tumor antigen-specific T-cell response, not the whole fragment C of tetanus toxoid is used as it is known to contain CD8⁺ T-cell epitopes. Two peptide sequences containing promiscuously binding helper epitopes were selected alternatively to ensure binding to as many MHC class II alleles as possible. Based on the data of the ex vivo studies the well-known epitopes p2 (QYIKANSKFIGITEL; TT₈₃₀₋₈₄₄) and p16 (MTNSVDDALINSTKIYSYFPSVISKVNQGAQG; TT₅₇₈₋₆₀₉) were selected. The p2 epitope was already used for peptide vaccination in clinical trials to boost anti-melanoma activity.

Present non-clinical data (unpublished) showed that RNA vaccines encoding both a tumor antigen plus promiscuously binding tetanus toxoid sequences lead to enhanced CD8⁺ T-cell responses directed against the tumor antigen and improved break of tolerance. Immunomonitoring data from patients vaccinated with vaccines including those sequences fused in frame with the tumor antigen-specific sequences reveal that the tetanus sequences chosen are able to induce tetanus-specific T-cell responses in almost all patients.

According to certain embodiments, an amino acid sequence which breaks immunological tolerance is fused, either directly or through a linker, e.g., a linker having the amino acid sequence GGSGGGGSGG, to the antigenic peptide or protein, i.e., CLDN6 (SEQ ID NO: 1), KK-LC-1 (SEQ ID NO: 5), MAGE-A3 (SEQ ID NO: 9), MAGE-A4 (SEQ ID NO: 13), PRAME (SEQ ID NO: 17), MAGE-C1 (SEQ ID NO: 21) or NY-ESO-1 (SEQ ID NO: 25), a variant thereof, or a fragment thereof.

Such amino acid sequences which break immunological tolerance are preferably located at the C-terminus of the antigenic peptide or protein (and optionally at the N-terminus of the amino acid sequence enhancing antigen processing and/or presentation, wherein the amino acid sequence which breaks immunological tolerance and the amino acid sequence enhancing antigen processing and/or presentation may be fused either directly or through a linker, e.g., a linker having the amino acid sequence GSSGGGGSPGGGSS), without being limited thereto. Amino acid sequences which break immunological tolerance as defined herein preferably improve T cell responses. In one embodiment, the amino acid sequence which breaks immunological tolerance as defined herein includes, without being limited thereto, sequences derived from tetanus toxoid-derived helper sequences p2 and p16 (P2P16), in particular a sequence comprising the amino acid sequence of SEQ ID NO: 33 or a functional variant thereof.

In one embodiment, an amino acid sequence which breaks immunological tolerance comprises the amino acid sequence of SEQ ID NO: 33, an amino acid sequence having at least 99%, 98%, 97%, 96%, 95%, 90%, 85%, or 80% identity to the amino acid sequence of SEQ ID NO: 33, or a functional fragment of the amino acid sequence of SEQ ID NO: 33, or the amino acid sequence having at least 99%, 98%, 97%, 96%, 95%, 90%, 85%, or 80% identity to the amino acid sequence of SEQ ID NO: 33. In one embodiment, an amino acid sequence which breaks immunological tolerance comprises the amino acid sequence of SEQ ID NO: 33.

Instead of using antigen RNAs fused with tetanus toxoid helper epitope, the tumor-antigen RNAs may be co-administered with a separate RNA coding for TT helper epitope during vaccination. Here, the TT helper epitope coding RNA may be added to each of the antigen-coding RNAs before preparation. In this way, mixed lipoplex nanoparticles are formed comprising both, antigen and helper epitope coding RNA in order to deliver both compounds to a given APC.

Accordingly, in some embodiments, compositions described herein may comprise RNA encoding Tetanus Toxoid-derived Helper Sequences p2 and p16 (P2P16). Likewise, methods described herein may comprise administration of RNA encoding Tetanus Toxoid-derived Helper Sequences p2 and p16 (P2P16).

Thus, a further aspect relates to a composition such as a pharmaceutical composition comprising particles such as lipoplex particles comprising:
(i) RNA encoding a vaccine antigen, and
(ii) RNA encoding: an amino acid sequence which breaks immunological tolerance.

Such composition is useful in a method of inducing an immune response against the vaccine antigen and thus, against a disease-associated antigen.

A further aspect relates to a method of inducing an immune response comprising administering particles such as lipoplex particles comprising:
(i) RNA encoding a vaccine antigen, and
(ii) RNA encoding: an amino acid sequence which breaks immunological tolerance.

In one embodiment, the amino acid sequence which breaks immunological tolerance comprises helper epitopes, preferably tetanus toxoid-derived helper epitopes.

In one embodiment, the RNA encoding a vaccine antigen is co-formulated as particles such as lipoplex particles with the RNA encoding an amino acid sequence which breaks immunological tolerance at a ratio of about 4:1 to about 16:1, about 6:1 to about 14:1, about 8:1 to about 12:1, or about 10:1.

According to certain embodiments, a signal peptide is fused, either directly or through a linker, e.g., a linker having the amino acid sequence GGSGGGGSGG, to the antigenic peptide or protein, e.g., MAGE-A3 (SEQ ID NO: 9), PRAME (SEQ ID NO: 17), MAGE-C1 (SEQ ID NO: 21) or NY-ESO-1 (SEQ ID NO: 25), a variant thereof, or a fragment thereof.

Such signal peptides are sequences, which typically exhibit a length of about 15 to 30 amino acids and are preferably located at the N-terminus of the antigenic peptide or protein, without being limited thereto. Signal peptides as defined herein preferably allow the transport of the antigenic peptide or protein as encoded by the RNA into a defined cellular compartment, preferably the cell surface, the endoplasmic reticulum (ER) or the endosomal-lysosomal compartment. In one embodiment, the signal peptide sequence as defined herein includes, without being limited thereto, the signal peptide sequence derived from the sequence encoding the human MHC class I complex (HLA-B51, haplotype A2, B27/B51, Cw2/Cw3), and preferably corresponds to the 78 bp fragment coding for the secretory signal peptide, which guides translocation of the nascent polypeptide chain into the endoplasmatic reticulum, and includes, in particular a sequence comprising the amino acid sequence of SEQ ID NO: 29 or a functional variant thereof.

In one embodiment, a signal sequence comprises the amino acid sequence of SEQ ID NO: 29, an amino acid sequence having at least 99%, 98%, 97%, 96%, 95%, 90%, 85%, or 80% identity to the amino acid sequence of SEQ ID NO: 29, or a functional fragment of the amino acid sequence of SEQ ID NO: 29, or the amino acid sequence having at least 99%, 98%, 97%, 96%, 95%, 90%, 85%, or 80% identity to the amino acid sequence of SEQ ID NO: 29. In one embodiment, a signal sequence comprises the amino acid sequence of SEQ ID NO: 29.

Such signal peptides are preferably used in order to promote secretion of the encoded antigenic peptide or protein. More preferably, a signal peptide as defined herein is fused to an encoded antigenic peptide or protein as defined herein.

Accordingly, in particularly preferred embodiments, the RNA described herein comprises at least one coding region encoding an antigenic peptide or protein and a signal peptide, said signal peptide preferably being fused to the antigenic peptide or protein, more preferably to the N-terminus of the antigenic peptide or protein as described herein.

According to certain embodiments, an amino acid sequence enhancing antigen processing and/or presentation is fused, either directly or through a linker to the antigenic peptide or protein, e.g., MAGE-A3 (SEQ ID NO: 9), MAGE-A4 (SEQ ID NO: 13), PRAME (SEQ ID NO: 17), MAGE-C1 (SEQ ID NO: 21) or NY-ESO-1 (SEQ ID NO: 25), a variant thereof, or a fragment thereof.

Such amino acid sequences which enhance antigen processing and/or presentation are preferably located at the C-terminus of the antigenic peptide or protein (and optionally at the C-terminus of the amino acid sequence breaking immunological tolerance, wherein the amino acid sequence which breaks immunological tolerance and the amino acid sequence enhancing antigen processing and/or presentation may be fused either directly or through a linker, e.g., a linker having the amino acid sequence GSSGGGGSPGGGSS), without being limited thereto. Amino acid sequences enhancing antigen processing and/or presentation as defined herein preferably improve antigen processing and presentation. In one embodiment, the amino acid sequence enhancing antigen processing and/or presentation as defined herein includes, without being limited thereto, sequences derived from the human MHC class I complex (HLA-B51, haplotype A2, B27/B51, Cw2/Cw3), in particular a sequence comprising the amino acid sequence of SEQ ID NO: 31 or a functional variant thereof.

In one embodiment, an amino acid sequence enhancing antigen processing and/or presentation comprises the amino acid sequence of SEQ ID NO: 31, an amino acid sequence having at least 99%, 98%, 97%, 96%, 95%, 90%, 85%, or 80% identity to the amino acid sequence of SEQ ID NO: 31, or a functional fragment of the amino acid sequence of SEQ ID NO: 31, or the amino acid sequence having at least 99%, 98%, 97%, 96%, 95%, 90%, 85%, or 80% identity to the amino acid sequence of SEQ ID NO: 31. In one embodiment, an amino acid sequence enhancing antigen processing and/or presentation comprises the amino acid sequence of SEQ ID NO: 31.

Such amino acid sequences enhancing antigen processing and/or presentation are preferably used in order to promote antigen processing and/or presentation of the encoded antigenic peptide or protein. More preferably, an amino acid sequence enhancing antigen processing and/or presentation as defined herein is fused to an encoded antigenic peptide or protein as defined herein.

Accordingly, in particularly preferred embodiments, the RNA described herein comprises at least one coding region encoding an antigenic peptide or protein and an amino acid sequence enhancing antigen processing and/or presentation, said amino acid sequence enhancing antigen processing and/or presentation preferably being fused to the antigenic peptide or protein, more preferably to the C-terminus of the antigenic peptide or protein as described herein.

In the following, embodiments of the vaccine RNAs are described, wherein certain terms used when describing elements thereof have the following meanings:
hAg-Kozak: 5′-UTR sequence of the human alpha-globin mRNA with an optimized ‘Kozak sequence’ to increase translational efficiency.
sec/MITD: Fusion-protein tags derived from the sequence encoding the human MHC class I complex (HLA-B51, haplotype A2, B27/B51, Cw2/Cw3), which have been shown to improve antigen processing and presentation. Sec corresponds to the 78 bp fragment coding for the secretory signal peptide, which guides translocation of the nascent polypeptide chain into the endoplasmatic reticulum. MITD corresponds to the transmembrane and cytoplasmic domain of the MHC class I molecule, also called MHC class I trafficking domain.
Antigen: Sequences encoding the respective tumor antigen.
Glycine-serine linker (GS): Sequences coding for short linker peptides predominantly consisting of the amino acids glycine (G) and serine (S), as commonly used for fusion proteins.
P2P16: Sequence coding for tetanus toxoid-derived helper epitopes to break immunological tolerance.
FI element: The 3′-UTR is a combination of two sequence elements derived from the “amino terminal enhancer of split” (AES) mRNA (called F) and the mitochondrial encoded 12S ribosomal RNA (called 1). These were identified by an ex vivo selection process for sequences that confer RNA stability and augment total protein expression.
A30L70: A poly(A)-tail measuring 110 nucleotides in length, consisting of a stretch of 30 adenosine residues, followed by a 10 nucleotide linker sequence and another 70 adenosine residues designed to enhance RNA stability and translational efficiency in dendritic cells.

In one embodiment, in particular in the case of CLDN6 (SEQ ID NO: 1) or KK-LC-1 (SEQ ID NO: 5), vaccine RNA described herein has the structure:
beta-S-ARCA(D1)-hAg-Kozak-Antigen-GS(2)-P2P16-FI-A30L70

In one embodiment, vaccine antigen described herein has the structure:
Antigen-GS(2)-P2P16

In one embodiment, in particular in the case of MAGE-A4 (SEQ ID NO: 13), vaccine RNA described herein has the structure:
beta-S-ARCA(D1)-hAg-Kozak-Antigen-GS(2)-P2P16-GS(3)-MITD-FI-A30L70

In one embodiment, vaccine antigen described herein has the structure:
Antigen-GS(2)-P2P16-GS(3)-MITD

In one embodiment, in particular in the case of MAGE-A3 (SEQ ID NO: 9), PRAME (SEQ ID NO: 17), MAGE-C1 (SEQ ID NO: 21) or NY-ESO-1 (SEQ ID NO: 25), vaccine RNA described herein has the structure:
beta-S-ARCA(D1)-hAg-Kozak-sec-GS(1)-Antigen-GS(2)-P2P16-GS(3)-MITD-FI-A30L70

In one embodiment, vaccine antigen described herein has the structure:
sec-GS(1)-Antigen-GS(2)-P2P16-GS(3)-MITD

In one embodiment, hAg-Kozak comprises the nucleotide sequence of SEQ ID NO: 35. In different embodiments, Antigen comprises an amino acid sequence selected from the group consisting of an amino acid sequence of CLDN6 (SEQ ID NO: 1), an amino acid sequence of KK-LC-1 (SEQ ID NO: 5), an amino acid sequence of MAGE-A3 (SEQ ID NO: 9), an amino acid sequence of MAGE-A4 (SEQ ID NO: 13), an amino acid sequence of PRAME (SEQ ID NO: 17), an amino acid sequence of MAGE-C1 (SEQ ID NO: 21) and an amino acid sequence of NY-ESO-1 (SEQ ID NO: 25). In one embodiment, see comprises the amino acid sequence of SEQ ID NO: 29. In the case of CLDN6, KK-LC-1 and MAGE-A4, an endogenous signal peptide is present, and thus, no further signal peptide needs to be added to SEQ ID NO: 1, 5 and 13. In one embodiment, P2P16 comprises the the amino acid sequence of SEQ ID NO: 33. In one embodiment, MITD comprises the the amino acid sequence of SEQ ID NO: 31. In one embodiment, GS(1) comprises the amino acid sequence GGSGGGGSGG. In one embodiment, GS(2) comprises the amino acid sequence GGSGGGGSGG. In one embodiment, GS(3) comprises the amino acid sequence GSSGGGGSPGGGSS. In one embodiment, Fl comprises the nucleotide sequence of SEQ ID NO: 36. In one embodiment, A30L70 comprises the nucleotide sequence of SEQ ID NO: 37.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable e.g. by translation of a truncated open reading frame that lacks the 3′-end of the open reading frame. A fragment shortened at the N-terminus (C-terminal fragment) is obtainable e.g. by translation of a truncated open reading frame that lacks the 5′-end of the open reading frame, as long as the truncated open reading frame comprises a start codon that serves to initiate translation. A fragment of an amino acid sequence comprises e.g. at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

By “variant” herein is meant an amino acid sequence that differs from a parent amino acid sequence by virtue of at least one amino acid modification. The parent amino acid sequence may be a naturally occurring or wild type (WT) amino acid sequence, or may be a modified version of a wild type amino acid sequence. Preferably, the variant amino acid sequence has at least one amino acid modification compared to the parent amino acid sequence, e.g., from 1 to about 20 amino acid modifications, and preferably from 1 to about 10 or from 1 to about 5 amino acid modifications compared to the parent.

By “wild type” or “WT” or “native” herein is meant an amino acid sequence that is found in nature, including allelic variations. A wild type amino acid sequence, peptide or protein has an amino acid sequence that has not been intentionally modified.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all mutants, splice variants, posttranslationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place.

Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

Preferably the degree of similarity, preferably identity between a given amino acid sequence and an amino acid sequence which is a variant of said given amino acid sequence will be at least about 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 100, at least about 120, at least about 140, at least about 160, at least about 180, or about 200 amino acids, preferably continuous amino acids. In preferred embodiments, the degree of similarity or identity is given for the entire length of the reference amino acid sequence.

“Sequence similarity” indicates the percentage of amino acids that either are identical or that represent conservative amino acid substitutions. “Sequence identity” between two amino acid sequences indicates the percentage of amino acids that are identical between the sequences.

In one embodiment, a fragment or variant of an amino acid sequence (peptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to antigens or antigenic sequences, one particular function is one or more immunogenic activities displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In one embodiment, the modifications in the amino acid sequence of the parent molecule or sequence do not significantly affect or alter the characteristics of the molecule or sequence. In different embodiments, the function of the functional fragment or functional variant may be reduced but still significantly present, e.g., immunogenicity of the functional variant may be at least 50%, at least 60%, at least 70%, at least 80%, or at least 90% of the parent molecule or sequence. However, in other embodiments, immunogenicity of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

A peptide and protein antigen described herein (CLDN6 protein, KK-LC-1 protein, MAGE-A3 protein, MAGE-A4 protein, PRAME protein, MAGE-C1 protein and NY-ESO-1 protein) when provided to a subject by administration of RNA encoding the antigen, i.e., a vaccine antigen, preferably results in stimulation, priming and/or expansion of T cells in the subject. Said stimulated, primed and/or expanded T cells are preferably directed against the target antigen, in particular the target antigen expressed by diseased cells, tissues and/or organs, i.e., the disease-associated antigen. Thus, a vaccine antigen may comprise the disease-associated antigen, or a fragment or variant thereof. In one embodiment, such fragment or variant is immunologically equivalent to the disease-associated antigen. In the context of the present disclosure, the term “fragment of an antigen” or “variant of an antigen” means an agent which results in stimulation, priming and/or expansion of T cells which stimulated, primed and/or expanded T cells target the disease-associated antigen, in particular when expressed on the surface of diseased cells, tissues and/or organs. Thus, the vaccine antigen administered according to the disclosure may correspond to or may comprise the disease-associated antigen, may correspond to or may comprise a fragment of the disease-associated antigen or may correspond to or may comprise an antigen which is homologous to the disease-associated antigen or a fragment thereof. If the vaccine antigen administered according to the disclosure comprises a fragment of the disease-associated antigen or an amino acid sequence which is homologous to a fragment of the disease-associated antigen said fragment or amino acid sequence may comprise an epitope of the disease-associated antigen or a sequence which is homologous to an epitope of the disease-associated antigen, wherein the T cells bind to said epitope. Thus, according to the disclosure, an antigen may comprise an immunogenic fragment of the disease-associated antigen or an amino acid sequence being homologous to an immunogenic fragment of the disease-associated antigen. An “immunogenic fragment of an antigen” according to the disclosure preferably relates to a fragment of an antigen which is capable of stimulating, priming and/or expanding T cells. It is preferred that the vaccine antigen (similar to the disease-associated antigen) provides the relevant epitope for binding by T cells. It is also preferred that the vaccine antigen (similar to the disease-associated antigen) is expressed on the surface of a cell such as an antigen-presenting cell so as to provide the relevant epitope for binding by the T cells. The vaccine antigen according to the invention may be a recombinant antigen.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, the term “immunologically equivalent” is preferably used with respect to the immunological effects or properties of antigens or antigen variants. For example, an amino acid sequence is immunologically equivalent to a reference amino acid sequence, if said amino acid sequence when exposed to T cells binding to the reference amino acid sequence or cells expressing the reference amino acid sequence induces an immune reaction having a specificity of reacting with the reference amino acid sequence, in particular stimulation, priming and/or expansion of T cells. Thus, a molecule which is immunologically equivalent to an antigen exhibits the same or essentially the same properties and/or exerts the same or essentially the same effects regarding the stimulation, priming and/or expansion of T cells as the antigen to which the T cells are targeted.

“Activation” or “stimulation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “priming” refers to a process wherein a T cell has its first contact with its specific antigen and causes differentiation into effector T cells.

The term “clonal expansion” or “expansion” refers to a process wherein a specific entity is multiplied. In the context of the present disclosure, the term is preferably used in the context of an immunological response in which lymphocytes are stimulated by an antigen, proliferate, and the specific lymphocyte recognizing said antigen is amplified. Preferably, clonal expansion leads to differentiation of the lymphocytes.

### Lipoplex Particles

RNA encoding a vaccine antigen may be administered formulated as particles, e.g., protein and/or lipid particles. In certain embodiments of the present disclosure, the RNA described herein may be present in RNA lipoplex particles. The RNA lipoplex particles and compositions comprising RNA lipoplex particles described herein are useful for delivery of RNA to a target tissue after parenteral administration, in particular after intravenous administration. The RNA lipoplex particles may be prepared using liposomes that may be obtained by injecting a solution of the lipids in ethanol into water or a suitable aqueous phase. In one embodiment, the aqueous phase has an acidic pH. In one embodiment, the aqueous phase comprises acetic acid, e.g., in an amount of about 5 mM. In one embodiment, the liposomes and RNA lipoplex particles comprise at least one cationic lipid and at least one additional lipid. In one embodiment, the at least one cationic lipid comprises 1,2-di-O-octadecenyl-3-trimethylammonium propane (DOTMA) and/or 1,2-dioleoyl-3-trimethylammonium-propane (DOTAP). In one embodiment, the at least one additional lipid comprises 1,2-di-(9Z-octadecenoyl)-sn-glycero-3-phosphoethanolamine (DOPE), cholesterol (Chol) and/or 1,2-dioleoyl-sn-glycero-3-phosphocholine (DOPC). In one embodiment, the at least one cationic lipid comprises 1,2-di-O-octadecenyl-3-trimethylammonium propane (DOTMA) and the at least one additional lipid comprises 1,2-di-(9Z-octadecenoyl)-sn-glycero-3-phosphoethanolamine (DOPE). In one embodiment, the liposomes and RNA lipoplex particles comprise 1,2-di-O-octadecenyl-3-trimethylammonium propane (DOTMA) and 1,2-di-(9Z-octadecenoyl)-sn-glycero-3-phosphoethanolamine (DOPE). Liposomes may be used for preparing RNA lipoplex particles by mixing the liposomes with RNA.

Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

### RNA Lipoplex Particle Diameter

RNA lipoplex particles described herein have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

In one embodiment, RNA lipoplex particles described herein exhibit a polydispersity index less than about 0.5, less than about 0.4, or less than about 0.3. By way of example, the RNA lipoplex particles can exhibit a polydispersity index in a range of about 0.1 to about 0.3.

### Lipid

In one embodiment, the lipid solutions, liposomes and RNA lipoplex particles described herein include a cationic lipid. As used herein, a “cationic lipid” refers to a lipid having a net positive charge. Cationic lipids bind negatively charged RNA by electrostatic interaction to the lipid matrix. Generally, cationic lipids possess a lipophilic moiety, such as a sterol, an acyl or diacyl chain, and the head group of the lipid typically carries the positive charge. Examples of cationic lipids include, but are not limited to 1,2-di-O-octadecenyl-3-trimethylammonium propane (DOTMA), dimethyldioctadecylammonium (DDAB); 1,2-dioleoyl-3-trimethylammonium propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2 (spermine carboxamide)ethyl]-N,N-dimethyl-1-propanamium trifluoroacetate (DOSPA). Preferred are DOTMA, DOTAP, DODAC, and DOSPA. In specific embodiments, the cationic lipid is DOTMA and/or DOTAP.

An additional lipid may be incorporated to adjust the overall positive to negative charge ratio and physical stability of the RNA lipoplex particles. In certain embodiments, the additional lipid is a neutral lipid. As used herein, a “neutral lipid” refers to a lipid having a net charge of zero. Examples of neutral lipids include, but are not limited to, 1,2-di-(9Z-octadecenoyl)-sn-glycero-3-phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, cholesterol, and cerebroside. In specific embodiments, the additional lipid is DOPE, cholesterol and/or DOPC.

In certain embodiments, the RNA lipoplex particles include both a cationic lipid and an additional lipid. In an exemplary embodiment, the cationic lipid is DOTMA and the additional lipid is DOPE. Without wishing to be bound by theory, the amount of the at least one cationic lipid compared to the amount of the at least one additional lipid may affect important RNA lipoplex particle characteristics, such as charge, particle size, stability, tissue selectivity, and bioactivity of the RNA. Accordingly, in some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

### Charge Ratio

The electric charge of the RNA lipoplex particles of the present disclosure is the sum of the electric charges present in the at least one cationic lipid and the electric charges present in the RNA. The charge ratio is the ratio of the positive charges present in the at least one cationic lipid to the negative charges present in the RNA. The charge ratio of the positive charges present in the at least one cationic lipid to the negative charges present in the RNA is calculated by the following equation: charge ratio=[(cationic lipid concentration (mol))*(the total number of positive charges in the cationic lipid)]/[(RNA concentration (mol))*(the total number of negative charges in RNA)]. The concentration of RNA and the at least one cationic lipid amount can be determined using routine methods by one skilled in the art.

In one embodiment, at physiological pH the charge ratio of positive charges to negative charges in the RNA lipoplex particles is from about 1.6:2 to about 1:2, or about 1.6:2 to about 1.1:2. In specific embodiments, the charge ratio of positive charges to negative charges in the RNA lipoplex particles at physiological pH is about 1.6:2.0, about 1.5:2.0, about 1.4:2.0, about 1.3:2.0, about 1.2:2.0, about 1.1:2.0, or about 1:2.0.

It has been found that RNA lipoplex particles having such charge ratio may be used to preferentially target spleen tissue or spleen cells such as antigen-presenting cells, in particular dendritic cells. Accordingly, in one embodiment, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs.

Thus, RNA lipoplex particles of the disclosure may be used for expressing RNA in such antigen presenting cells. In one embodiment, the antigen presenting cells are dendritic cells and/or macrophages.

### A. Salt and Ionic Strength

According to the present disclosure, the compositions described herein may comprise salts such as sodium chloride. Without wishing to be bound by theory, sodium chloride functions as an ionic osmolality agent for preconditioning RNA prior to mixing with the at least one cationic lipid. Certain embodiments contemplate alternative organic or inorganic salts to sodium chloride in the present disclosure. Alternative salts include, without limitation, potassium chloride, dipotassium phosphate, monopotassium phosphate, potassium acetate, potassium bicarbonate, potassium sulfate, potassium acetate, disodium phosphate, monosodium phosphate, sodium acetate, sodium bicarbonate, sodium sulfate, sodium acetate, lithium chloride, magnesium chloride, magnesium phosphate, calcium chloride, and sodium salts of ethylenediaminetetraacetic acid (EDTA).

Generally, compositions comprising RNA lipoplex particles described herein comprise sodium chloride at a concentration that preferably ranges from 0 mM to about 500 mM, from about 5 mM to about 400 mM, or from about 10 mM to about 300 mM. In one embodiment, compositions comprising RNA lipoplex particles comprise an ionic strength corresponding to such sodium chloride concentrations.

### B. Stabilizer

Compositions described herein may comprise a stabilizer to avoid substantial loss of the product quality and, in particular, substantial loss of RNA activity during freezing, lyophilization, spray-drying or storage such as storage of the frozen, lyophilized or spray-dried composition.

In an embodiment the stabilizer is a carbohydrate. The term “carbohydrate”, as used herein refers to and encompasses monosaccharides, disaccharides, trisaccharides, oligosaccharides, and polysaccharides.

In embodiments of the disclosure, the stabilizer is mannose, glucose, sucrose or trehalose. According to the present disclosure, the RNA lipoplex particle compositions described herein have a stabilizer concentration suitable for the stability of the composition, in particular for the stability of the RNA lipoplex particles and for the stability of the RNA.

### C. pH and Buffer

According to the present disclosure, the RNA lipoplex particle compositions described herein have a pH suitable for the stability of the RNA lipoplex particles and, in particular, for the stability of the RNA. In one embodiment, the RNA lipoplex particle compositions described herein have a pH from about 5.5 to about 7.5.

According to the present disclosure, compositions that include buffer are provided. Without wishing to be bound by theory, the use of buffer maintains the pH of the composition during manufacturing, storage and use of the composition. In certain embodiments of the present disclosure, the buffer may be sodium bicarbonate, monosodium phosphate, disodium phosphate, monopotassium phosphate, dipotassium phosphate, [tris(hydroxymethyl)methylamino]propanesulfonic acid (TAPS), 2-(Bis(2-hydroxyethyl)amino)acetic acid (Bicine), 2-Amino-2-(hydroxymethyl)propane-1,3-diol (Tris), N-(2-Hydroxy-1,1-bis(hydroxymethyl)ethyl)glycine (Tricine), 3-[[1,3-dihydroxy-2-(hydroxymethyl)propan-2-yl]amino]-2-hydroxypropane-1-sulfonic acid (TAPSO), 2-[4-(2-hydroxyethyl)piperazin-1-yl]ethanesulfonic acid (HEPES), 2-[[1,3-dihydroxy-2-(hydroxymethyl)propan-2-yl]amino]ethanesulfonic acid (TES), 1,4-piperazinediethanesulfonic acid (PIPES), dimethylarsinic acid, 2-morpholin-4-ylethanesulfonic acid (MES), 3-morpholino-2-hydroxypropanesulfonic acid (MOPSO), or phosphate buffered saline (PBS). Other suitable buffers may be acetic acid in a salt, citric acid in a salt, boric acid in a salt and phosphoric acid in a salt.

In one embodiment, the buffer is HEPES.

In one embodiment, the buffer has a concentration from about 2.5 mM to about 15 mM.

### D. Chelating Agent

Certain embodiments of the present disclosure contemplate the use of a chelating agent. Chelating agents refer to chemical compounds that are capable of forming at least two coordinate covalent bonds with a metal ion, thereby generating a stable, water-soluble complex. Without wishing to be bound by theory, chelating agents reduce the concentration of free divalent ions, which may otherwise induce accelerated RNA degradation in the present disclosure. Examples of suitable chelating agents include, without limitation, ethylenediaminetetraacetic acid (EDTA), a salt of EDTA, desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), diethylenetriaminepentaacetic acid (DTPA), bis(aminoethyl)glycolether-N,N,N′,N′-tetraacetic acid, iminodiacetic acid, citric acid, tartaric acid, fumaric acid, or a salt thereof. In certain embodiments, the chelating agent is EDTA or a salt of EDTA. In an exemplary embodiment, the chelating agent is EDTA disodium dihydrate.

In some embodiments, the EDTA is at a concentration from about 0.05 mM to about 5 mM.

### E. Physical State of Compositions of the Disclosure

In embodiments, the composition of the present disclosure is a liquid or a solid. Non-limiting examples of a solid include a frozen form or a lyophilized form. In a preferred embodiment, the composition is a liquid.

In some embodiments, the composition of the present disclosure comprises RNA encoding vaccine antigen as described herein, a buffering agent, such as HEPES, a cationic lipid, such as DOTMA, a helper lipid, such as DOPE, a stabilizer, such as EDTA, an osmolality agent, such as sodium chloride, a cryoprotectant, such as sucrose, and a solvent, such as water for injection. In some embodiments, the cationic lipid, such as DOTMA, and the helper lipid, such as DOPE, complex the RNA. In some embodiments, the cationic lipid, such as DOTMA, and the helper lipid, such as DOPE, form RNA lipoplex particles with the RNA. In some embodiments, the composition of the present disclosure comprises RNA encoding vaccine antigen as described herein, HEPES, DOTMA, DOPE, EDTA, sodium chloride, sucrose, and water for injection.

### Pharmaceutical Compositions of the Disclosure

The agents described herein may be administered in pharmaceutical compositions or medicaments and may be administered in the form of any suitable pharmaceutical composition. In one embodiment, the pharmaceutical composition described herein is an immunogenic composition for inducing an immune response against lung cancer in a subject.

For example, in one embodiment, the immunogenic composition is a vaccine.

In one embodiment of all aspects of the invention, the components described herein such as RNA encoding a vaccine antigen may be administered in a pharmaceutical composition which may comprise a pharmaceutically acceptable carrier and may optionally comprise one or more adjuvants, stabilizers etc. In one embodiment, the pharmaceutical composition is for therapeutic or prophylactic treatments, e.g., for use in treating or preventing lung cancer.

The RNA described herein, e.g., formulated as RNA lipoplex particles, is useful as or for preparing pharmaceutical compositions or medicaments for therapeutic or prophylactic treatments.

The compositions of the present disclosure may be administered in the form of any suitable pharmaceutical composition.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation. In the context of the present disclosure, the pharmaceutical composition comprises the RNA described herein, e.g., formulated as RNA lipoplex particles. The pharmaceutical compositions of the present disclosure preferably comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cyctokines, such as monokines, lymphokines, interleukins, chemokines. The chemokines may be IL-1, IL-2, IL-3, IL-4, IL-5, IL-6, IL-7, IL-8, IL-9, IL-10, IL-12, INFa, INF-γ, GM-CSF, LT-a. Further known adjuvants are aluminium hydroxide, Freund's adjuvant or oil such as Montanide® ISA51. Other suitable adjuvants for use in the present disclosure include lipopeptides, such as Pam3Cys.

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

In some embodiments, an effective amount comprises an amount sufficient to cause a tumor/lesion to shrink. In some embodiments, an effective amount is an amount sufficient to decrease the growth rate of a tumor (such as to suppress tumor growth). In some embodiments, an effective amount is an amount sufficient to delay tumor development. In some embodiments, an effective amount is an amount sufficient to prevent or delay tumor recurrence. In some embodiments, an effective amount is an amount sufficient to increase a subject's immune response to a tumor, such that tumor growth and/or size and/or metastasis is reduced, delayed, ameliorated, and/or prevented. An effective amount can be administered in one or more administrations. In some embodiments, administration of an effective amount (e.g., of a composition comprising mRNAs) may: (i) reduce the number of cancer cells; (ii) reduce tumor size; (iii) inhibit, retard, slow to some extent and may stop cancer cell infiltration into peripheral organs; (iv) inhibit (e.g., slow to some extent and/or block or prevent) metastasis; (v) inhibit tumor growth; (vi) prevent or delay occurrence and/or recurrence of tumor; and/or (vii) relieve to some extent one or more of the symptoms associated with the cancer.

The pharmaceutical compositions of the present disclosure may contain salts, buffers, preservatives, and optionally other therapeutic agents. In one embodiment, the pharmaceutical compositions of the present disclosure comprise one or more pharmaceutically acceptable carriers, diluents, and/or excipients.

Suitable preservatives for use in the pharmaceutical compositions of the present disclosure include, without limitation, benzalkonium chloride, chlorobutanol, paraben, and thimerosal. The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid, or solid suspension and/or mixing media. Examples of suitable diluents include ethanol, glycerol, and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In one embodiment, the pharmaceutical composition of the present disclosure includes isotonic saline.

Pharmaceutically acceptable carriers, excipients, or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

### Routes of Administration of Pharmaceutical Compositions of the Disclosure

In one embodiment, pharmaceutical compositions described herein may be administered intravenously, intraarterially, subcutaneously, intradermally, intranodullary or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the pharmaceutical composition is formulated for systemic administration. In another preferred embodiment, the systemic administration is by intravenous administration.

### Use of Pharmaceutical Compositions of the Disclosure

The RNA described herein, e.g., formulated as RNA lipoplex particles, may be used in the therapeutic or prophylactic treatment of diseases in which provision of amino acid sequences encoded by the RNA to a subject results in a therapeutic or prophylactic effect.

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

In the present context, the term “treatment”, “treating”, or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g., mouse, rat, rabbit, dog, cat, cattle, swine, sheep, horse, or primate) that can be afflicted with or is susceptible to a disease or disorder (e.g., cancer) but may or may not have the disease or disorder. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In one embodiment of the disclosure, the aim is to provide an immune response against cancer cells expressing one or more tumor antigens, and to treat a cancer disease involving cells expressing one or more tumor antigens. In one embodiment, the cancer is lung cancer. In one embodiment, the cancer is non-small cell lung cancer, e.g., advanced or metastasized non-small cell lung cancer, such as non-squamous and squamous cell carcinoma. In one embodiment, the cancer is unresectable Stage Ill or metastatic Stage IV NSCLC. In one embodiment, the tumor antigens are CLDN6, KK-LC-1, MAGE-A3, MAGE-A4, PRAME, and optionally one or both of MAGE-C1 and NY-ESO-1.

A pharmaceutical composition comprising RNA may be administered to a subject to elicit an immune response against one or more antigens or one or more epitopes encoded by the RNA in the subject which may be therapeutic or partially or fully protective. A person skilled in the art will know that one of the principles of immunotherapy and vaccination is based on the fact that an immunoprotective reaction to a disease is produced by immunizing a subject with an antigen or an epitope, which is immunologically relevant with respect to the disease to be treated. Accordingly, pharmaceutical compositions described herein are applicable for inducing or enhancing an immune response. Pharmaceutical compositions described herein are thus useful in a prophylactic and/or therapeutic treatment of a disease involving an antigen or epitope, in particular lung cancer.

As used herein, “immune response” refers to an integrated bodily response to an antigen or a cell expressing an antigen and refers to a cellular immune response and/or a humoral immune response. A cellular immune response includes, without limitation, a cellular response directed to cells expressing an antigen and being characterized by presentation of an antigen with class I or class II MHC molecule. The cellular response relates to T lymphocytes, which may be classified as helper T cells (also termed CD4⁺ T cells) that play a central role by regulating the immune response or killer cells (also termed cytotoxic T cells, CD8⁺ T cells, or CTLs) that induce apoptosis in infected cells or cancer cells. In one embodiment, administering a pharmaceutical composition of the present disclosure involves stimulation of an anti-tumor CD8⁺ T-cell response against cancer cells expressing one or more tumor antigens. In a specific embodiment, the tumor antigens are presented with class I MHC molecule.

The present disclosure contemplates an immune response that may be protective, preventive, prophylactic, and/or therapeutic. As used herein, “induces [or inducing] an immune response” may indicate that no immune response against a particular antigen was present before induction or it may indicate that there was a basal level of immune response against a particular antigen before induction, which was enhanced after induction. Therefore, “induces [or inducing] an immune response” includes “enhances [or enhancing] an immune response”. The term “immunotherapy” relates to the treatment of a disease or condition by inducing, or enhancing an immune response. The term “immunotherapy” includes antigen immunization or antigen vaccination.

The terms “immunization” or “vaccination” describe the process of administering an antigen to an individual with the purpose of inducing an immune response, for example, for therapeutic or prophylactic reasons.

In one embodiment, the present disclosure envisions embodiments wherein RNA lipoplex particles as described herein targeting spleen tissue are administered. The RNA encodes a peptide or protein comprising an antigen or an epitope as described, for example, herein. The RNA is taken up by antigen-presenting cells in the spleen such as dendritic cells to express the peptide or protein. Following optional processing and presentation by the antigen-presenting cells an immune response may be generated against the antigen or epitope resulting in a prophylactic and/or therapeutic treatment of a disease involving the antigen or epitope. In one embodiment, the immune response induced by the RNA lipoplex particles described herein comprises presentation of an antigen or fragment thereof, such as an epitope, by antigen presenting cells, such as dendritic cells and/or macrophages, and activation of cytotoxic T cells due to this presentation. For example, peptides or proteins encoded by the RNAs or procession products thereof may be presented by major histocompatibility complex (MHC) proteins expressed on antigen presenting cells. The MHC peptide complex can then be recognized by immune cells such as T cells or B cells leading to their activation.

Thus, in one embodiment the RNA in the RNA lipoplex particles described herein, following administration, is delivered to the spleen and/or is expressed in the spleen. In one embodiment, the RNA lipoplex particles are delivered to the spleen for activating splenic antigen presenting cells. Thus, in one embodiment, after administration of the RNA lipoplex particles RNA delivery and/or RNA expression in antigen presenting cells occurs. Antigen presenting cells may be professional antigen presenting cells or non-professional antigen presenting cells. The professional antigen presenting cells may be dendritic cells and/or macrophages, even more preferably splenic dendritic cells and/or splenic macrophages. Accordingly, the present disclosure relates to RNA lipoplex particles or a pharmaceutical composition comprising RNA lipoplex particles as described herein for inducing or enhancing an immune response, preferably an immune response against lung cancer.

In one embodiment, systemically administering RNA lipoplex particles or a pharmaceutical composition comprising RNA lipoplex particles as described herein results in targeting and/or accumulation of the RNA lipoplex particles or RNA in the spleen and not in the lung and/or liver. In one embodiment, RNA lipoplex particles release RNA in the spleen and/or enter cells in the spleen. In one embodiment, systemically administering RNA lipoplex particles or a pharmaceutical composition comprising RNA lipoplex particles as described herein delivers the RNA to antigen presenting cells in the spleen. In a specific embodiment, the antigen presenting cells in the spleen are dendritic cells or macrophages.

The term “macrophage” refers to a subgroup of phagocytic cells produced by the differentiation of monocytes. Macrophages which are activated by inflammation, immune cytokines or microbial products nonspecifically engulf and kill foreign pathogens within the macrophage by hydrolytic and oxidative attack resulting in degradation of the pathogen. Peptides from degraded proteins are displayed on the macrophage cell surface where they can be recognized by T cells, and they can directly interact with antibodies on the B-cell surface, resulting in T- and B-cell activation and further stimulation of the immune response. Macrophages belong to the class of antigen presenting cells. In one embodiment, the macrophages are splenic macrophages.

The term “dendritic cell” (DC) refers to another subtype of phagocytic cells belonging to the class of antigen presenting cells. In one embodiment, dendritic cells are derived from hematopoietic bone marrow progenitor cells. These progenitor cells initially transform into immature dendritic cells. These immature cells are characterized by high phagocytic activity and low T-cell activation potential. Immature dendritic cells constantly sample the surrounding environment for pathogens such as viruses and bacteria. Once they have come into contact with a presentable antigen, they become activated into mature dendritic cells and begin to migrate to the spleen or to the lymph node. Immature dendritic cells phagocytose pathogens and degrade their proteins into small pieces and upon maturation present those fragments at their cell surface using MHC molecules. Simultaneously, they upregulate cell-surface receptors that act as co-receptors in T-cell activation such as CD80, CD86, and CD40 greatly enhancing their ability to activate T cells. They also upregulate CCR7, a chemotactic receptor that induces the dendritic cell to travel through the blood stream to the spleen or through the lymphatic system to a lymph node. Here they act as antigen-presenting cells and activate helper T cells and killer T cells as well as B cells by presenting them antigens, alongside non-antigen specific co-stimulatory signals. Thus, dendritic cells can actively induce a T-cell- or B-cell-related immune response. In one embodiment, the dendritic cells are splenic dendritic cells.

The term “antigen presenting cell” (APC) is a cell of a variety of cells capable of displaying, acquiring, and/or presenting at least one antigen or antigenic fragment on (or at) its cell surface. Antigen-presenting cells can be distinguished in professional antigen presenting cells and non-professional antigen presenting cells.

The term “professional antigen presenting cells” relates to antigen presenting cells which constitutively express the Major Histocompatibility Complex class II (MHC class II) molecules required for interaction with naive T cells. If a T cell interacts with the MHC class II molecule complex on the membrane of the antigen presenting cell, the antigen presenting cell produces a co-stimulatory molecule inducing activation of the T cell. Professional antigen presenting cells comprise dendritic cells and macrophages.

The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

“Antigen processing” refers to the degradation of an antigen into procession products, which are fragments of said antigen (e.g., the degradation of a protein into peptides) and the association of one or more of these fragments (e.g., via binding) with MHC molecules for presentation by cells, such as antigen presenting cells to specific T cells.

The term “disease involving an antigen” or “disease involving an epitope” refers to any disease which implicates an antigen or epitope, e.g., a disease which is characterized by the presence of an antigen or epitope. The disease involving an antigen or epitope can be a cancer disease or simply cancer. As mentioned above, the antigen may be a disease-associated antigen, such as a tumor-associated antigen and the epitope may be derived from such antigen.

The terms “cancer disease” or “cancer” refer to or describe the physiological condition in an individual that is typically characterized by unregulated cell growth. Examples of cancers include, but are not limited to, carcinoma, lymphoma, blastoma, sarcoma, and leukemia. More particularly, examples of such cancers include bone cancer, blood cancer lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. One particular form of cancer that can be treated by the compositions and methods described herein is lung cancer. In one embodiment, the cancer is non-small cell lung cancer, e.g., advanced or metastasized non-small cell lung cancer, such as non-squamous and squamous cell carcinoma. In one embodiment, the cancer is unresectable Stage Ill or metastatic Stage IV NSCLC. The term “cancer” according to the disclosure also comprises cancer metastases.

Specific embodiments of Treatments of the Disclosure

In one embodiment, RNA described herein, e.g., formulated as RNA lipoplex particles, is administered by intravenous (IV) injection.

In one embodiment, RNA described herein, e.g., formulated as RNA lipoplex particles, is administered at a dose of between 20 μg and 200 μg, e.g., between 30 μg and 100 μg, for example between 60 μg and 90 μg. For example, RNA described herein, may be administered at a dose of about 30 μg, 40 μg, 50 μg, 60 μg, 70 μg, 80 μg, or 90 μg.

In one embodiment, RNA described herein, e.g., formulated as RNA lipoplex particles, comprises RNA encoding MAGEA3, RNA encoding CLDN6, RNA encoding KK-LC-1, RNA encoding PRAME, RNA encoding MAGE-A4, and RNA encoding MAGE-C1 in equimolar amounts.

In one embodiment, a treatment described herein comprises one or more cycles. In one embodiment, a treatment described herein comprises multiple cycles, e.g., 3 or more cycles, 4 or more cycles, 5 or more cycles, 6 or more cycles, 7 or more cycles, 8 or more cycles, 9 or more cycles, 10 or more cycles, 11 or more cycles, 12 or more cycles, 13 or more cycles, 14 or more cycles, or 15 or more cycles. In one embodiment, the length of a cycle is between 14 and 28 days, e.g., about 21 days.

In one embodiment, a treatment described herein comprises one or more cycles, e.g., 2 cycles, wherein RNA described herein, e.g., formulated as RNA lipoplex particles, is administered several times on different days of a cycle. For example, the length of a cycle may be 21 days and the RNA may be administered on days 1, 8 and 15 of a cycle.

In one embodiment, a treatment described herein comprises one or more cycles, e.g., 2, 3, 4, 5, 6, 7, 8, 9, 10 or even more cycles, wherein RNA described herein, e.g., formulated as RNA lipoplex particles, is administered only on a single day of a cycle. For example, the length of a cycle may be 21 days and the RNA may be administered on day 1 of a cycle.

In one embodiment, a treatment described herein comprises multiple cycles, comprising one or more cycles, e.g., 2 cycles, wherein RNA described herein, e.g., formulated as RNA lipoplex particles, is administered several times on different days of a cycle (e.g., the length of a cycle may be 21 days and the RNA may be administered on days 1, 8 and 15 of a cycle), followed by one or more cycles, e.g., 2, 3, 4, 5, 6, 7, 8, 9, 10 or even more cycles, wherein RNA described herein, e.g., formulated as RNA lipoplex particles, is administered only on a single day of a cycle (e.g., the length of a cycle may be 21 days and the RNA may be administered on day 1 of a cycle).

In one embodiment, patients receive RNA on Days 1, 8 and 15 of Cycles 1 and 2, and from Cycle 3 onwards, RNA is administered on Day 1 only. In this embodiment, the amount of RNA on Day 1 of Cycle 1 may be 60 μg, and the amount of RNA on all subsequent applications (Cycle 1 Days 8 and 15, Cycle 2 Days 1, 8 and 15 and from Cycle 3 onwards) may be 90 μg.

In one embodiment, the RNA described herein is administrered as monotherapy. In one embodiment, prior therapy included, if eligible, at least one PD 1/PD-L1 inhibitor and one platinum-based chemotherapy regimen, and one additional systemic therapy.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: Identification of a set of immunogenic targets for use in treating non-small cell lung cancer

The scope of our preclinical research focused on two objectives: (1) identification of a set of valuable immunogenic targets in non-small cell lung cancer; (2) selection of appropriate cancer patients with a high probability for target-specific immune reaction and therapy benefit upon vaccination.

In an initial target discovery approach, RNA sequencing data of non-small cell lung cancer and healthy tissues was explored in order to select for the most frequently and tumor-specifically expressed target genes. These targets should be expressed in a significant number of tumors, weakly expressed or absent in essential organs like brain and heart, and lower expressed compared to tumors or absent in other human tissues except of reproductive or gynecological tissues. Selection and filtering of genes based on above-mentioned criteria aims at enlarging the probability that the target can induce immunogenicity (not recognized as self-antigen) with a limited toxicity (not represented in essential organs). Targets were evaluated for the two main subtypes of non-small cell lung cancer, lung adenocarcinoma and squamous cell carcinoma, and finally selected to address both disease subtypes.

All in silico analyses were done using publicly available (GTEx, Genotype-Tissue Expression project (Nature genetics 45, 580-585 (2013)) and TCGA, The Cancer Genome Atlas (Nature 489, 519-525 (2012); Campbell, J. D. et al., Nat. Genet. 48, 607-616 (2016); Nature 511, 543-550 (2014)) and proprietary RNA-Seq gene expression data. RNA reads were aligned to the hg19 reference genome and transcriptome, and gene expression was determined by comparison with UCSC known genes transcript and exon coordinates, followed by normalization to RPKM units (Mortazavi, A. et al., Nature methods 5, 621-628 (2008); Langmead, B. et al., Genome biology 10, R25; (2009)). Targets were selected by comparing expression in tumor and normal tissues, and to achieve a high coverage across the tumor cohorts. Target-expressing tumors were defined by expression value >1 rpkm.

For qRT-PCR analysis using the Fluidigm Biomark™ Platform 164 fresh frozen primary lung cancer tissue samples were used. In total 91 fresh frozen normal tissue samples from 43 different tissue types were used for qRT-PCR analysis. RNA was isolated from tissues using the Qiagen RNeasy Lipid Tissue Mini Kit according to the manufacturer's instructions. RNA was converted to cDNA by first strand cDNA synthesis using the TAKARA-PrimeScript™ RT Reagent Kit with gDNA Eraser according to the manufacturer's instructions. qRT-PCR analysis using the Fluidigm detection system was done according to the manufacturer's instructions. After normalization to the housekeeping genes HPRT1, HMBS, and TBP, relative RNA expression was quantified using AMCt calculation. A calibrator of 18.2 corresponding to 30 (maximal number of cycles used in the PCR) minus the mean of the HPRT1 housekeeping gene value of the normal tissue samples was used in this analysis. Primers used in the analysis are listed in Table 1. Technical replicates, including different cDNA syntheses were summarized by using the median expression values. Relative expression of the gene of interest in normal tissue reveals the median expression value, if more than one tissue sample of the same tissue type was analyzed. Target-expressing tumors were defined by specific cutoffs dependent on the expression intensities in critical normal tissues (Table 1).

TABLE 1
Oligonucleotides used for qRT-PCR analysis

| | | | Expression |
|---|---|---|---|
| Tran- | Forward Primer | Reverse Primer | cutoff |
| script | Sequence | Sequence | (a.u.) |
| CLDN6 | ACTCGGCCTAGGAA | CAGAGGCCATGGCG | 3000 |
| | TTTCCCT | AGG | |
| KK-LC-1 | GTGTGCCTTGATTG | CCTGGCTATTGAGT | 1000 |
| | TCTTCTGG | GTGGG | |
| MAGEA3* | CGGTGAGGAGGCAA | AATGGAGACCCACT | 1000 |
| | GGTTC | GGCAGAT | |
| MAGEA4 | GCACCAAAGGAGAA | TGTCAAGGCCTTCC | 1000 |
| | GATCTGCC | TCAGGCT | |
| MAGEC1 | GACCTATCCAGTCT | AGGGGGTTTGTCTT | 1000 |
| | TCAAGGTGC | CTGGGA | |
| NY-ESO-1 | CGTGCCAGGGGTGC | TCTGCAGCAGTCAG | 1000 |
| | TTCT | TCGGATAG | |
| PRAME | GCAGTATATCGCCC | GGTTCATCACGTGC | 30000 |
| | AGTTCAC | CTGAGC | |
| HPRT1 | TGACACTGGCAAAA | GGTCCTTTTCACCA | N/A |
| | CAATGCA | GCAAGCT | |
| HMBS | AGCCCAGCTGCAGA | GGATGATGGCACTG | N/A |
| | GAAAGT | AACTCC | |
| TBP | GAGCCAAGAGTGAA | GCTCCCCACCATAT | N/A |
| | GAACAGTC | TCTGAATCT | |

*Primers for MAGEA3 also detect MAGEA5 and MAGEA6 transcripts because of a very high sequence homology.

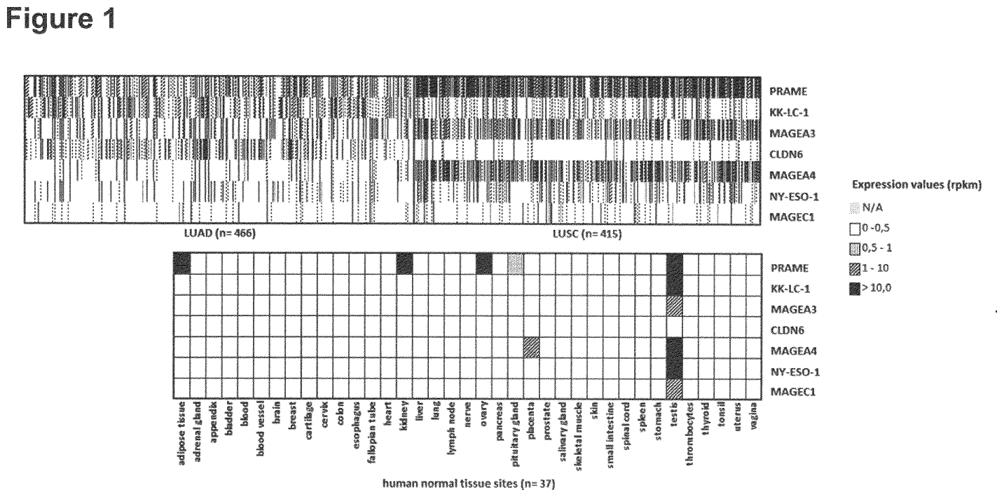

Expression Analysis of NSCLC target genes in Tumor and Normal Tissue using RNA-Seq Data Public and in-house generated RNA-Seq gene expression data from 3809 normal tissue samples, 881 non-small-cell lung carcinoma (NSCLC) samples including 466 lung adenocarcinoma (LUAD) and 415 squamous cell lung carcinoma (LUSC) samples were used to generate expression heatmaps (FIG. 1). For most of the targets strong RNA expression was detected in a large fraction of NSCLC tissues, but only in few normal tissues like testis and placenta. Except of testis and placenta, PRAME RNA expression was also detected in adrenal gland, kidney, ovary and pituitary gland that should be carefully monitored in future clinical studies.

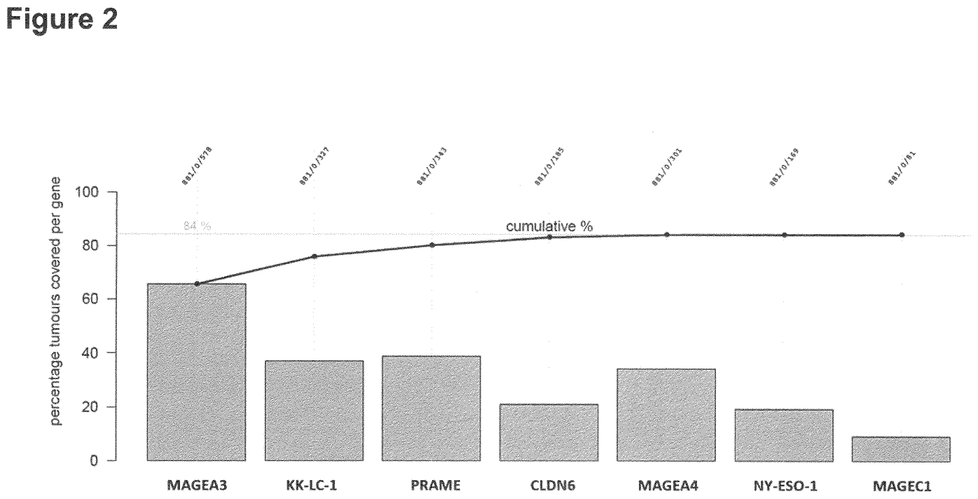

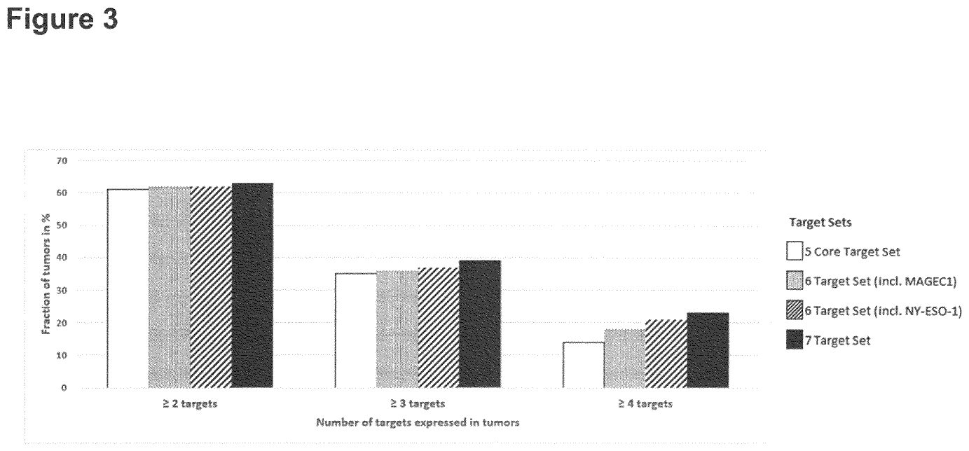

In order to calculate the percentage of NSCLC patient that can be potentially addressed by a vaccine approach tumor percentage was calculated for individual targets, as well as for cumulative coverage in target combinations (FIG. 2). For example, MAGEA3 alone was expressed in 66% of tumors. The coverage increased with additional four targets up to 84% of tumors expressing one or more targets. In order to test the added value of the target MAGEC1 and NY-ESO-1, the fraction of tumors were calculated expressing at least two, three or more targets (FIG. 3). Tumor fractions increased in sets with higher number of targets e.g. about 10% more tumors expressing four and more targets that indicated added value of targets like MAGEC1, NY-ESO-1 or both.

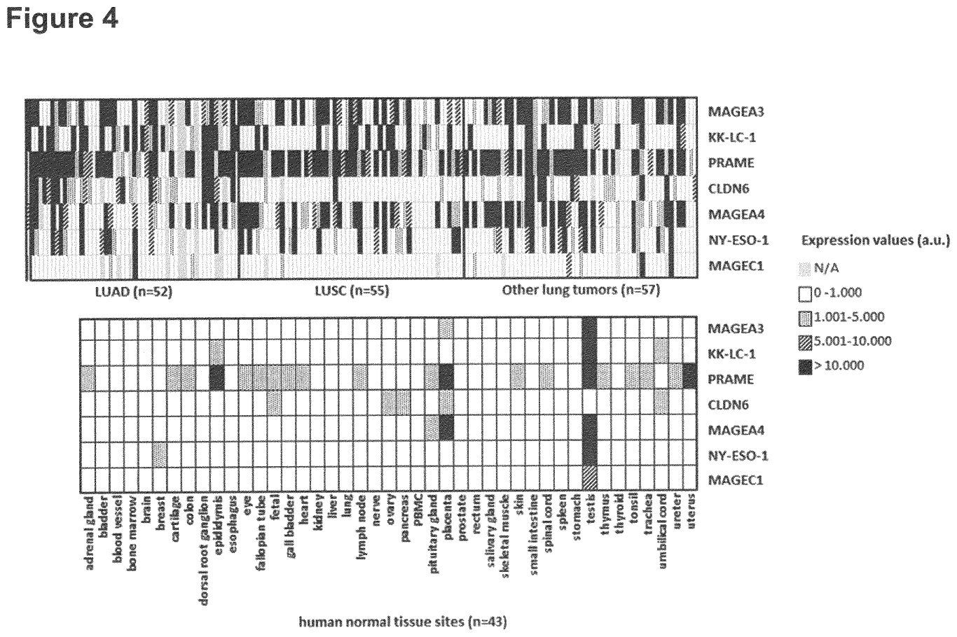

Expression Analysis of NSCLC target genes in Tumor and Normal Tissue using qRT-PCR data In order to confirm the expression of targets in NSCLC and normal tissue using an independent method and patient cohort, qRT-PCR analyses were performed using the Fluidigm Biomark™ platform. RNA expression intensities of 164 NSCLC and other lung tumors, and 43 normal tissue sites were used to generate expression heatmaps (FIG. 4). Strong RNA expression was detected in many lung tumor tissues, but only in few normal tissues like testis and placenta, epididymis and uterus.

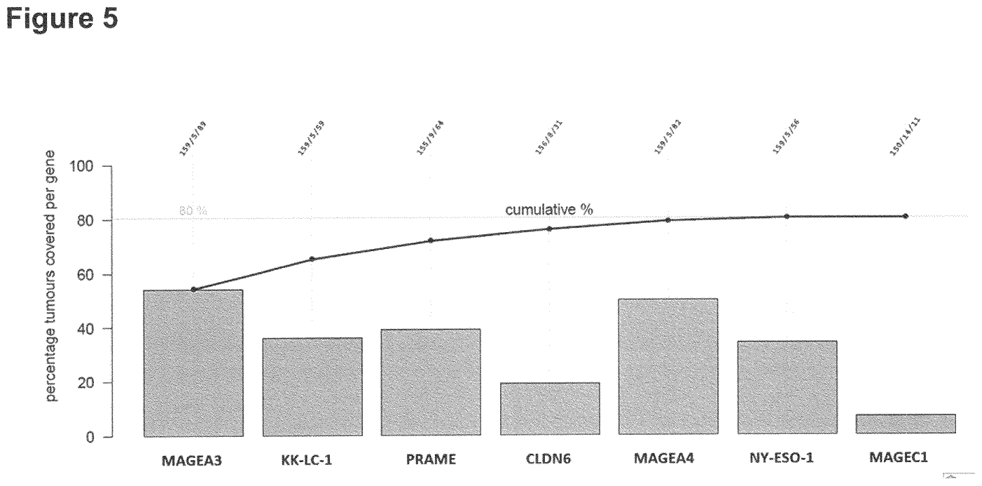

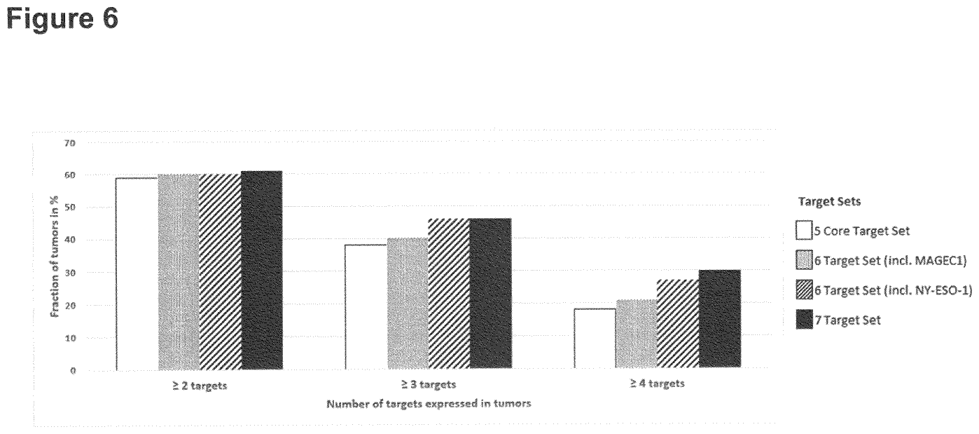

In order to calculate the percentage of NSCLC patient that can be potentially addressed by a vaccine approach tumor percentage was calculated for individual targets, as well as for cumulative coverage in target combinations (FIG. 5). For example, MAGEA3 alone was expressed in 56% of tumors. The coverage increased with additional four targets up to 80% of tumors expressing one or more targets. In order to test the added value of the target MAGEC1 and NY-ESO-1, the fraction of tumors were calculated expressing at least two, three or more targets (FIG. 6). Tumor fractions increased in sets with higher number of targets e.g. about 10% more tumors expressing four and more targets that indicated added value of targets like MAGEC1, NY-ESO-1 or both.

### Conclusion

The objective of the present study was to investigate and select immunotherapy targets. By comparing transcriptome data of normal and tumor tissues the antigens KK-LC-1, MAGEA3, PRAME, MAGEA4, CLDN6, MAGEC1 and NY-ESO-1 were selected as targets for the development of a recombinant RNA vaccine against non-small cell lung cancer.

RNA-Seq data and qRT-PCR data indicated abundant expression of the seven targets in both subtypes, lung adenocarcinoma and squamous cell carcinoma. Based on the tumor fraction expressing at least one of seven targets up to four of five patients with NSCLC might benefit from a vaccination approach. Vaccination of the targets MAGEC1 and NY-ESO-1 less frequently expressed in tumors might have a benefit in the context of an increased fraction of patients expressing two or more targets and because of previously observed immunogenicity. The transcription profile in normal tissues of most of the targets did not indicate a risk for severe organ toxicity upon vaccination.

### Example 2: In Vivo Induction of Antigen-Specific T Cells

The aim of the present study was to confirm the in vivo induction of antigen-specific T cells by the RNA batches coding for MAGEA3, KK-LC-1, CLDN6, NY-ESO-1, MAGEA4, and PRAME produced under GMP-conditions mentioned above as well as to assess immunogenicity of in vitro transcribed RNA coding for MAGEC1 produced under R&D conditions. RNAs were tested in vivo in mice using intravenously (i.v.) injected liposome-formulated RNA-LPX. The antigen sequences are embedded into processing and presentation enhancing domains. At the N-terminus of the resulting protein, the company constructed a secretory domain to facilitate translocation into the ribosome, while at the C-terminus, the transmembrane domain and the cytoplasmic part of a human MHC-molecule are fused in-frame to enhance MHC-class II presentation. For the present experiments, transgenic A2/DR1 mice engineered to express the human HLA-A*0201 and HLA-DRB1*01 molecules, but no endogenous, i.e. murine, MHC class I and class II molecules were used to examine the induction of T cells reactive against HLA-restricted epitopes. The A2/DR1 mouse resembles a model to display T-cell immunogenicity and most frequent human MHC-alleles.

Human MHC-transgenic mice (A2/DR1 mice) were used to examine the generation of T cells reactive against HLA-restricted epitopes in vivo. Seven groups of three to five mice were immunized three or four times on days 1, 8, 15, and 22 by i.v. injection of RNA-LPX coding for the above-mentioned antigens using the liposomes shown below. The animals were euthanized after a further five days either on day 20 or day 27 and spleens were removed to prepare a single-cell suspension of splenocytes. The immunogenicity of the used RNA-LPX was tested using splenocytes restimulated with pools of respective peptides. In case of MAGEC1 RNA, the immunogenicity was tested using splenocytes restimulated with bone marrow-derived dendritic cells (BMDCs) electroporated with in vitro transcribed MAGEC1 RNA. IFN-γ secretion of specific T cells was determined by ELISPOT assays. ConA was used as a positive control to test for the functionality of the assay. As negative controls, medium only and irrelevant peptides or in vitro transcribed RNA coding for irrelevant antigen that are not recognized by the T cells were used.

### Test Item

Liposomes for RNA-LPX formulation
Designation: L1
Content: 1.8 mg/mL DOTMA and 1.0 mg/mL DOPE
Designation: L2
Content: 1.8 mg/mL DOTMA and 1.0 mg/mL DOPE
Designation: L3
Content: 0.4 mg/mL DOTMA and 0.23 mg/mL DOPE
   In vitro transcribed RNA
Designation: MAGEA3
Concentration: 0.04 mg RNA/mL
Designation: KK-LC-1
Concentration: 0.5 mg RNA/mL
Designation: CLDN6
Concentration: 0.5 mg RNA/mL
Designation: NY-ESO-1
Concentration: 0.5 mg RNA/mL
Designation: MAGEA4
Concentration: 0.5 mg RNA/mL
Designation: PRAME
Concentration: 0.5 mg RNA/mL
Designation: MAGEC1

For the preparation of RNA-LPX, the test items were thawed, and all reagents were brought to ambient temperature (15-25° C.). All materials were RNase-free. RNA stock solution, water, 1.5 mM NaCl, and liposomes were used to inject up to five mice (200 μL/mouse) including one surplus mouse. A vial with RNA was prepared, water was added and the diluted RNA was vortexed, after which 1.5 M NaCl was added followed by additional vortexing. Liposomes were added to the resulting mixture to obtain the respective amount of isotonic solution of RNA-LPX with a charge ratio of 1.3:2 (liposome:RNA), and the tube was inverted two to four times and incubated for 10 minutes at ambient temperature. The resulting solution was a slightly opaque RNA-LPX dispersion. The particle size in the resulting RNA-LPX dispersions was investigated by photon-correlation spectroscopy.

### Test System

Species: mouse
Strain: HLA-A2.1+/+/HLA-DR1+/+ double transgenic, H-2 class I (β2m0)-/class II (IA βb0)-KO
mice
Breeder: Animal Facility, BioNTech SE
Sex: male/female
Age: 6-41 weeks
Number of animals: 33

### Animal Care

### General Information

Mice were bred in BioNTech SE's animal facility as specified in section 0. All experiments and protocols were approved by the local authorities (animal welfare testing authorization —Rhineland-Palatinate Regulatory Authority No. 23 177-07/G 14-12-088), conducted according to the FELASA recommendations and in compliance with EC Directive 2010/63/EU. Only animals with an unobjectionable health status were selected for testing procedures. With the aid of the lab animal colony management system PyRAT (Scionics Computer Innovation GmbH, Dresden, Germany), each animal was registered upon arrival or birth and tracked until euthanasia. Each cage was labelled with a cage card indicating the mouse strain, gender, date of birth, and number of animals per cage. At the start of the experiment, additional information was added, including the project and license numbers, the start of the experiment, and details on interventions. Where necessary for identification, animals were arbitrarily numbered with ear marks.

### Housing Condition and Husbandry

Mice were housed in BioNTech SE's animal facility under barrier and specific pathogen free conditions in individually ventilated cages (Sealsafe GM500 IVC Green Line, TECNIPLAST, HohenpeiBenberg, Germany; 500 cm²) with a maximum of five animals per cage. The temperature and relative humidity in the cages and animal unit were kept at 20 to 24° C. and 45 to 55%, respectively, and the air changed in the cages at a rate of 75 changes/h. The cages with dust-free bedding made of debarked chopped aspen wood (Abedd LAB & VET Service GmbH, Vienna, Austria, product code: LTE E-001) were changed weekly. Autoclaved ssniff M-Z food (sniff Spezialdiaten GmbH, Soest, Germany; product code: V1124) and autoclaved tap water were provided ad libitum and changed at least once weekly. All materials were autoclaved prior to use.

### Animal Monitoring and Observations

Routine animal monitoring was performed daily and included inspection for dead animals and control of food and water supplies. Each animal's health was closely assessed at least once weekly regarding body weight, fur condition, activity, body temperature, behavior, clinical signs, automutilation, signs of fighting and breathing.

### Endpoint of Experiment/Termination Criteria

Animals were euthanized in accordance with § 4 of the German animal welfare act and the recommendation of GV-SOLAS by cervical dislocation. The study was terminated on day 21 or 27 of the experiment.

### Treatment Schedule, Route of Administration, and Dose

TABLE 2
Experimental setup.

| | | | | | | Number |
|---|---|---|---|---|---|---|
| | Treatment | | Schedule | Dose per | | of |
| Group | RNA | Liposome | [dosing days] | treatment [μg] | Route | animals |
| 1 | MAGEA3 | L3 | 1, 8, 15 | 2 | i.v. | 5 |
| 2 | KK-LC-1 | L2 | 1, 8, 15, 22 | 30 | i.v. | 5 |
| 3 | CLDN6 | L2 | 1, 8, 15, 22 | 30 | i.v. | 5 |
| 4 | NY-ESO | L1 | 1, 8, 15, 22 | 30 | i.v. | 3 |
| 5 | MAGEA4 | L2 | 1, 8, 15, 22 | 30 | i.v. | 5 |
| 6 | PRAME | L2 | 1, 8, 15, 22 | 30 | i.v. | 5 |
| 7 | MAGEC1 | L2 | 1, 8, 15, 22 | 30 | i.v. | 5 |

The respective test-item formulations were injected retro-orbitally under isoflurane anesthesia in a fixed volume of 200 μL.

### Sample Collection and Processing

### Splenocytes

Spleens were removed after euthanization and single cell suspensions were prepared as follows: The removed organs are pressed through a 70 μm cell mesh using the plunger of a syringe to release the cells from the organ into a tube. After washing with PBS the cell pellet is incubated with erythrocyte lysis buffer, washed in PBS and passed again through a 70 μm cell mesh. In the end the cells are resuspended in medium and counted.

### IFN-γ ELISPOT Assay

The IFN-γ ELISPOT assay was used to measure IFN-γ release of in vitro re-stimulated T cells as an indicator for the induction of antigen-specific T cells.

### Preparation of Peptides

After delivery, all peptides were dissolved in cell culture grade DMSO to a final concentration of 1-2 mg/mL. From this peptide solution, either 2 or 4 μL (4 μg) were transferred into a 1.5 mL tube and filled to 1,000 μL with media to give a final concentration of 4 μg/mL. 100 μL of peptide solution were pipetted into each well containing 100 μL of single splenocyte single cell suspension (final concentration of peptides per well: 2 μg/mL).

### Splenocytes

Isolated splenocytes at 5×10⁵ cells/well in 200 μL were stimulated in a 96-well plate using peptide pools (15 mers, overlapping by 11 amino acids; 2 μg/mL) spanning the respective human proteins for approximately 20 h at 37 ° C. As peptide controls, splenocytes were incubated with 2 μg/mL tetanus toxoid derived peptides (P2, P16, and P17) as well as irrelevant CMV peptide. In case of MAGEC1 RNA, splenocytes stimulation was performed using electroporated cultivated mouse BMDCs isolated form bone marrow. Cultivated mouse BMDCs were electroporated with antigen-coding RNA for MAGEC1 and as negative control with RNAs coding for GAGEC2. A total of 5×10⁴ electroporated BMDCs/well in 100 μL were incubated with 5×10⁵ splenocytes for 20 h at 37° C. For all groups, splenocytes were incubated with medium alone as a negative control or with 2 μg/mL ConA as an internal positive control test, illustrating the functionality of the assay. Spot numbers were counted and analyzed using the ImmunoSpot® S5 Versa ELISPOT Analyzer, the ImmunoCapture™ Image Acquisition software, and the ImmunoSpot® Analysis software Version 5 (C.T.L.; Cellular Technologies Ltd.).

### Results

In splenocytes obtained from immunized A2/DR1 mice five days after the final RNA-LPX injections, the in vivo induction of antigen-specific T cells was determined by ELISPOT analysis. To test for the immunogenicity RNA, splenocytes were re-stimulated with a peptide pool (15 mers, overlapping by 11 amino acids) spanning the respective human proteins or electroporated BMDCs.

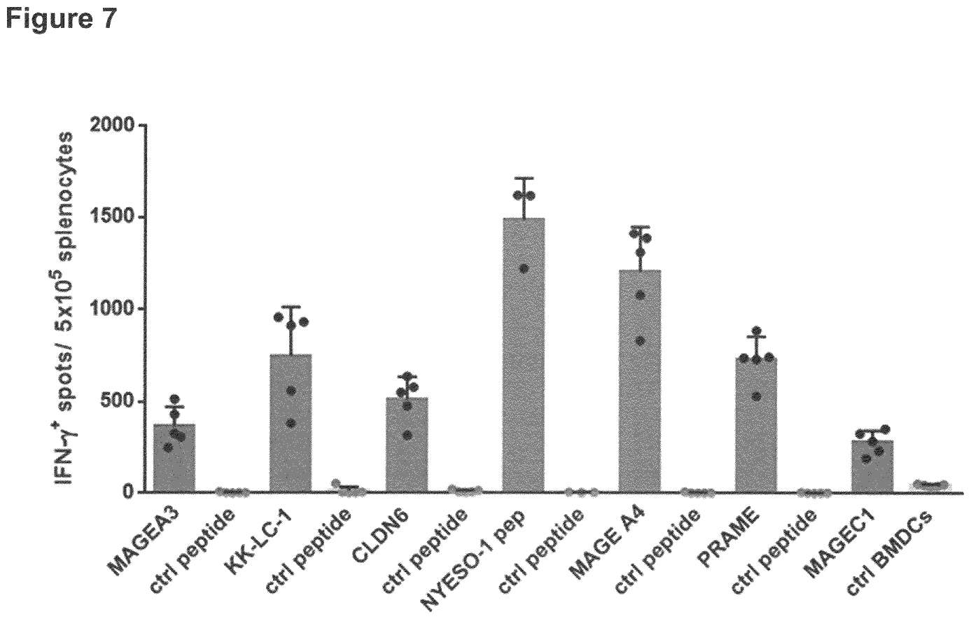

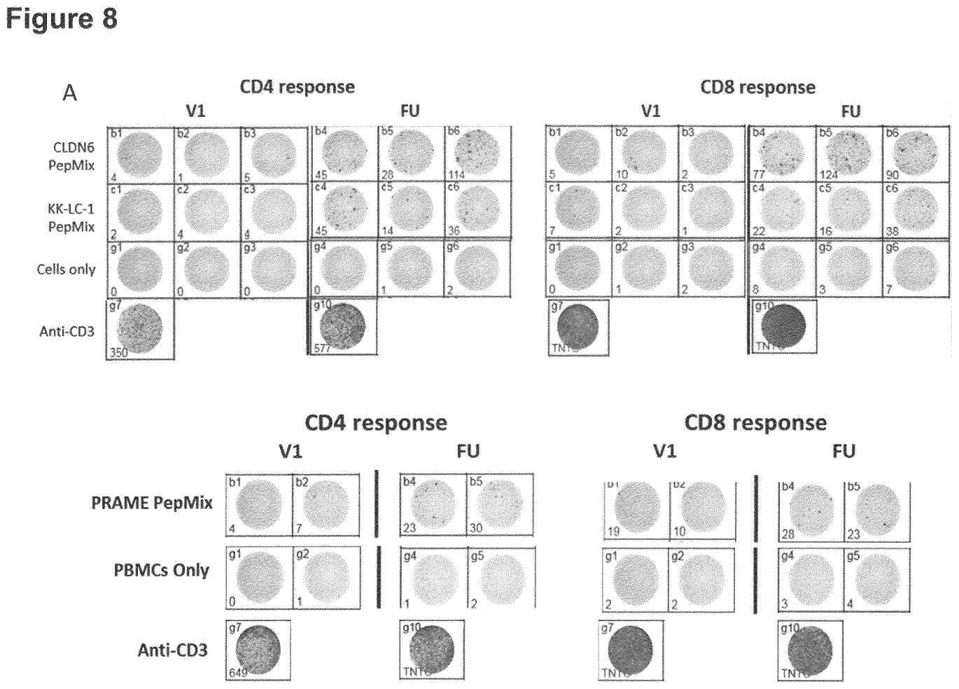

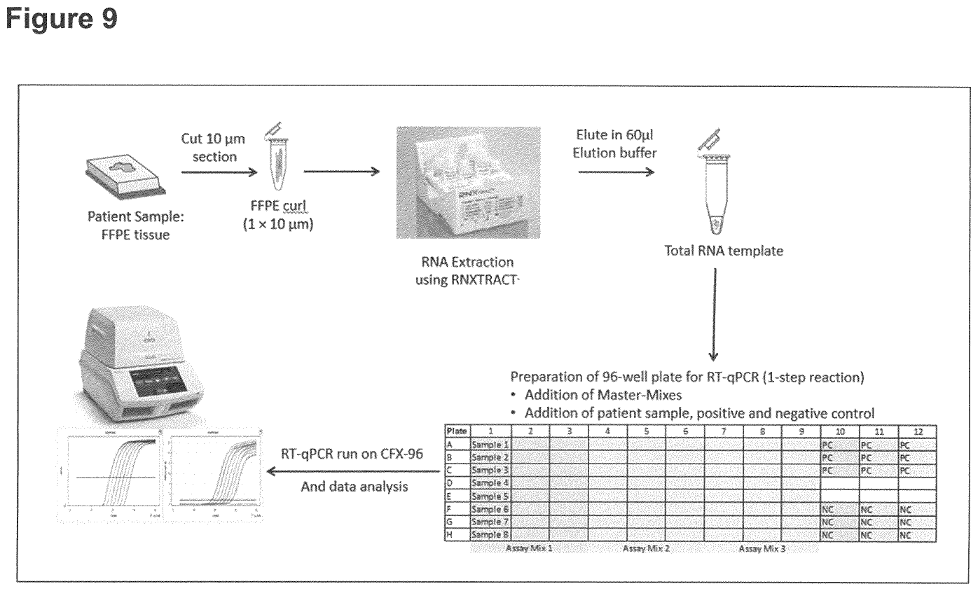

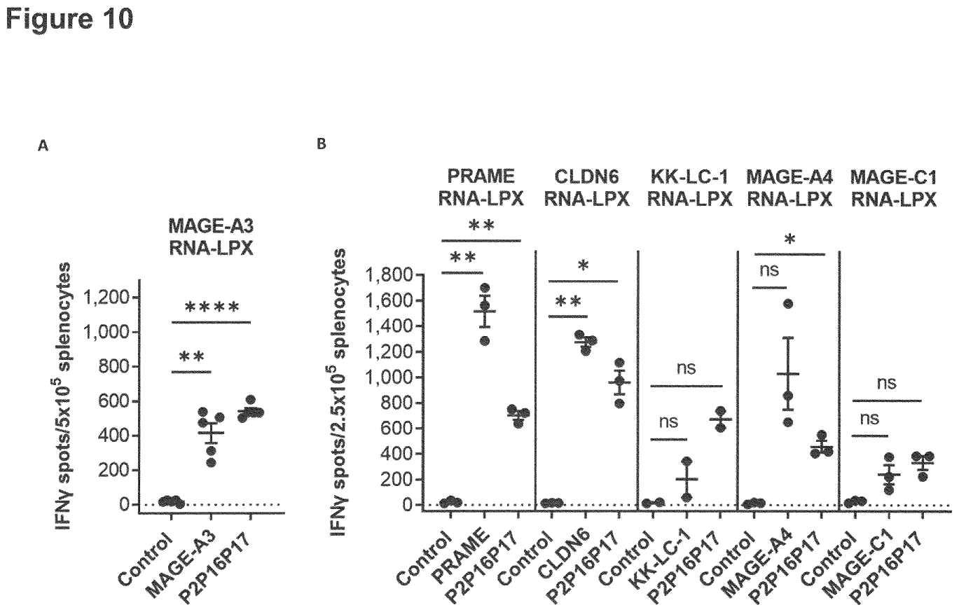

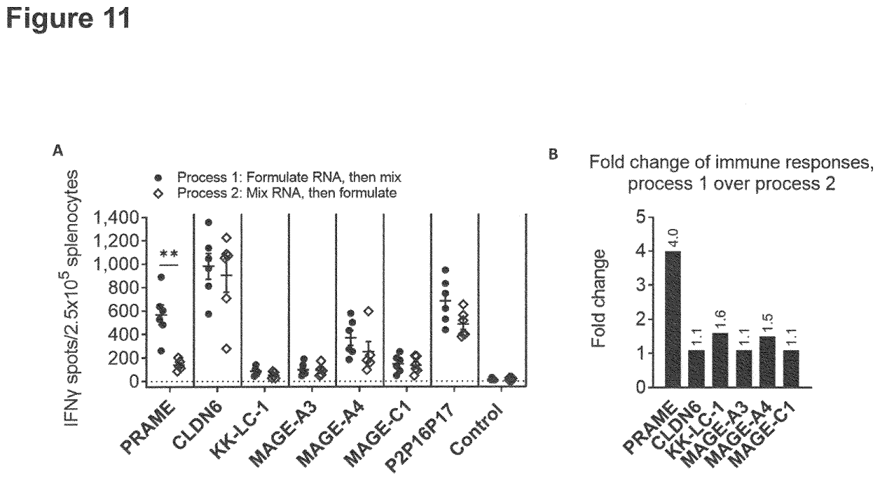

Restimulation of splenocytes with a peptide pool or electroporated BMDCs from mice immunized with resulted in specific immunogenicity (FIG. 7). IFN-γ⁺ spot count induced by MAGEA3-, KK-LC-1-, CLDN6-, NY-ESO-1-, MAGEA4-, PRAME- and MAGEC1-coding RNA-LPX was significantly higher compared to restimulation with respective controls. On all ELISPOT plates, the negative control medium alone induced only a minimal spot count regardless of the animal the splenocytes had been derived from; ConA as a positive control induced high numbers of spots in the ELISPOT assays confirming the presence of functional T cells in the isolated splenocytes, as expected.

### Conclusion

This study was designed to confirm immunogenicity of lung cancer antigens MAGEA3, KK-LC-1, CLDN6, NY-ESO-1, MAGEA4, and PRAME manufactured for use in clinical trials and MAGEC1 RNA produced under R&D conditions. The obtained data demonstrate that all production batches are immunogenic in a human HLA-A02 background in A2/DR1 mice. In summary, the data suggest that all seven RNAs can be utilized in an immunotherapeutic approach to induce antigen-specific T cells in patients.

### Example 3: In Vivo Induction of Antigen-Specific T Cells

To determine the immunogenicity of the RNA encoded tumor-associated antigens (TAAs) we analyzed T-cell responses in pre- and post-vaccination blood samples of patients using IFNγ-ELISPOT assay.

IFNγ ELISpot

Multiscreen filter plates (Merck Millipore), pre-coated with antibodies specific for IFNγ (ELISpotPro kit Mabtech) were washed with PBS and blocked with X-VIVO 15 (Lonza) containing 2% human serum albumin (CSL-Behring) for 1-5 hours. For analysis of ex vivo T-cell responses, 3×10⁵ cells/well CD4- or CD8-depleted PBMCs plus 3×10⁴ CD8⁺ or CD4⁺ T-cells/well were used as CD8 and CD4 effectors, respectively. Tests were performed in triplicate or duplicate and included positive and negative controls, i.e. PBMCs incubated with anti-CD3 and with medium alone, respectively. Spots were visualized with a secondary antibody directly conjugated with ExtrAvidin Alkaline Phosphatase ALP and BCIP/NBT substrate (ELISpotPro kit, Mabtech). Plates were scanned using an AID Classic Robot ELISPOT Reader and analyzed by AID ELISPOT 7.0 software.

For patient WO5YAH treated with vaccines de novo vaccine-induced CD4⁺ and CD8⁺ T cell responses against KKLC1 and CLDN6 were detected in post treatment samples by ex vivo ELISPOT analysis (FIG. 8A). For patient AW8VMT treated with vaccines, de novo vaccine-induced CD4⁺ and CD8*T-cell responses against PRAME were detected in post-treatment samples by ex vivo ELISPOT analysis (FIG. 8B).

### Example 4: Survey of Tumor-Associated Antigens (TAA) Using RT-qPCR in Combination with PD-L1 Expression Analysis Using Immunohistochemistry Staining in a Retrospective Cohort of Clinical Tumor Samples of Patients with Non-Small Cell Lung Cancer

### Background, Objectives, Study Design

Targets for the lung cancer project were selected based on several sources that used different methods to assess gene expression. While TCGA (The Cancer Genome Atlas) aggregated RNASeq data generated by high throughput NGS, RT-qPCR (Reverse transcription-quantitative real-time polymerase chain reaction) was used on smaller cohorts to verify the findings with more sensitive and specific assays. The datasets showed different TAA (Tumor-associated antigen) frequency profiles and coverages in the evaluated subtypes, which are within the expectable margin of variation for the datasets. These datasets did not include data beyond the expression of the selected TAAs. To strengthen our data, a cohort of ˜200 samples was obtained from clinical routine and analysed for TAA as well as PD-L1 expression. Mutational data was collected from the clinical data.

The objective of the study was to generate data on TAA and PD-L1 expression in a set of lung adenocarcinoma, lung squamous cell carcinoma and large cell neuroendocrine carcinoma samples. Key question was the percent coverage of samples analyzed with the selected TAAs (at least one expressed) in general and in combination with PD-L1 expression and/or driver mutations.

The study intended to collect initially 200 samples, thereof as many large cell neuroendocrine carcinoma (LCNEC) samples as possible and equal numbers of lung adenocarcinoma (LUAD) and lung squamous cell carcinoma (LUSC) samples. Where possible, metastases paired with primary tumor samples assessing differences in TAA expression were analyzed. For all samples, PD-L1 expression was assessed using an IHC IVD. Samples were for coverage with the selected TAAs in groups defined by subtype and/or biomarker profile.

### MATERIALS AND METHODS

Reverse transcription-quantitative real-time polymerase chain reaction (RT-qPCR) In One step RT-qPCR, reverse transcription and qPCR are combined in one reaction. A specific reverse transcription happens using the specific reverse primers and later PCR amplification proceeds using DNA polymerase. For the analysis, a master mix is prepared containing assay mix, enzyme mix (includes reverse transcriptase, DNA polymerase, buffer and dNTPs) and water. The master mix is dispensed into the wells of a 96 well plate, and RNA sample and appropriate controls (PC and NC) are added. The test includes three triplex assay mixes containing three individual assays/targets. For the detection hydrolysis probe technology is adopted using different fluorescence dyes (FAM, HEX, ATTO647N) to distinguish the assays of the triplex reaction. RT-qPCR is performed on BioRad's CFX96 instrument. RT-qPCR runs for patient analysis will later only use reagents of one kit lot (and not mix reagents of different kit lots).

PD-L1 immunohistochemistry

In this process, patient's tumor samples are provided on sections and used for PD-L1 analyses. The sections are labelled for PD-L1 staining (PD-L1 (SP263) Assay (Ventana)) with the Benchmark conform labels including the patient ID and Biosampling ID. After the staining process is completed all slides will be labelled with printed Datamatrix code labels to mark the sections.

Stained sections are digitalized (Whole slide imaging) for storage using a section scanner as detailed in SOP-010-165_Axio Scan.Z1-Slidescanner or a comparable device. In addition, an image of the section label is saved as part of the digitalized slide file. The digitalized section images are automatically assigned a file name that corresponds to the section ID Based on the stained section the PD-L1 scoring of the sample is determined by a pathologist. In a semi-quantitative analysis, the percentage of the tumor proportion score (TPS: Number of tumor cells/number of vital tumor cells), Immune cell score (IC+: Tumor associated immune cells with PD-L1 staining) and combined positivity score (CPS: Number of positive tumor cells, lymphocytes and macrophages/number of viable cells ×100) should be estimated stained patient section.

### Test Item

Formalin-fixed and paraffin-embedded (FFPE) tissue samples were subjected to sectioning. Sections of 3 μm were mounted on glass slides for IHC analyses and 10 μm sections (“curls”) were placed into microliter tubes for subsequent isolation of nucleic acids.

Cohort size was aimed at 200 specimens. LCNEC and metastases and their primary tumors if available were prioritized. Cohort was to be filled with equal numbers of LUSC and LUAD samples. The final cohort was composed of 170 primary tumor specimens and 18 metastasis specimens. A total of 4 specimens returned an invalid measurement, due to insufficient amplifiable RNA quantities (2 primary, 2 metastatic samples). Some subtypes were not within our scope and were excluded from individual analysis. The relevant cohort was thus composed of 74 lung adeno carcinoma (LUAD) primary and 13 LUAD metastases, 59 lung squamous cell carcinoma (LUSC) specimens and 26 large cell neuroendocrine carcinoma (LCNEC) specimens.

Groups (primary/metastasis by subtype) smaller than 10 specimens were not considered for conclusions.

The following materials and devices were used:

| Item | ID | Supplier / ArticleNo | Lot# / Serial # |
|---|---|---|---|
| BioRad CFX96 | 109-01 | BioRad / 1854096 | Base Serial Number |
| | | (discontinued) | CT023866 |
| | | | Optical Head Serial |
| | | | Number 785BR15721 |
| BioRad CFX96 | 109-02 | BioRad / 1854096 | Base Serial Number |
| | | (discontinued) | CT025007 |
| | | | Optical Head Serial |
| | | | Number 785BR19388 |
| CFX Manager Software | n/A | BioRad / bundled with device | Version 3.1.1517.0823 |
| Nuclease-free water | 00383 | Thermo Fisher Scientific / | 2009041 |
| | | AM9939 (Ambion) | |
| UltraPlex ™ 1-Step | 00650 | VWR, 733-2848 (Quanta) | 66175074 |
| ToughMix ® (4X) | | | 66171686 |
| Assay Mix 1 Lot 3a | AM1_MAGEA4_{—} | BioNTech Diagnostics, MolDx | 210104DaWe01 |
| | MAGEC1_CALM2 | | |
| Assay Mix 2 Lot 3a | AM2_CT83_{—} | BioNTech Diagnostics, MolDx | 210104DaWe01 |
| | PRAME_HUWE1 | | |
| Assay Mix 3 Lot3a | AM3_CLDN6_{—} | BioNTech Diagnostics, MolDx | 210127DaWe03 |
| | MAGEA3_MRPL19 | | |
| Human total | 00408 | Agilent / 750500 | 201230DaWe02(6404812) |
| reference RNA | | | (Lot 3) |
| Positive control (PC) | NA | BioNTech Diagnostics, MolDx | 210104DaWe02 (Lot 3a) |

Analysis of gene expression by RT-qPCR

RNA extracted from patient FFPE tumor samples is analyzed for mRNA expression of the tumor-associated antigens CLDN6, CT83, MAGEA3, MAGEA4, MAGEC1 and PRAME. For this purpose, gene-specific RT-qPCR assays have been developed and will be used for R&D analysis.

### RNA Extraction

The established preanalytical process starts with the extraction of total RNA from 1×10 rn FFPE tissue sections using the RNXtract® RNA Extraction Kit (BioNTech Diagnostics GmbH) according to the IFU. In brief, tissue sections are depaffinized by heating to 80° C. in aqueous buffer and subsequently lysed using Proteinase K. RNA is then bound to beads under facilitating buffer conditions, beads are fixed by magnetic force before removing supernantant at each step from binding, during each of the three washing steps and also at final elution.

### RT-qPCR

The extracted RNA is analyzed via one-step RT-qPCR, where the mRNA is first reverse transcribed into complementary DNA (cDNA) and then amplified by qPCR, using gene- and isoform-specific primers and probes. The RT-qPCR is performed on the CFX96 instrument (Bio-Rad). Established positive and negative controls (PC and NC) are analyzed within each RT-qPCR run to determine the validity of the run and also—in case of the PC- to function as a calibrator in analysis. Only valid runs are analyzed. The RT-qPCR assays are established as triplex assays allowing analysis of three targets per reaction. The assays within one reaction are distinguished by different fluorescent dyes of the probes (FAM, HEX and ATTO647N, see table below). Primary analysis output is a quantification cycle (Cq) value for each target, which is the point where the signal crosses a defined threshold above background signal. The Cq is a measure for the amount of target molecules in the sample before PCR amplification. Triplicate measurements are performed for each assay per sample, and the median Cq is used for calculations. For each sample, it has to be determined whether sufficient analyte (=amplifiable RNA) is present for analysis. For this, the expression levels of three reference genes (RGs): CALM2, HUWE1 and MRPL19, are used as surrogate for the RNA amount. The mean of the median Cqs of the three RGs is calculated, from here on named “Combined Reference” (CombRef), which also serves for normalization of target gene expression against different RNA input amounts. For this, the CombRef is subtracted from the median Cq of the target to obtain the normalized, relative expression of each target=delta Cq (dCq). To compensate for inter-run and inter-instrument variations, the dCq of the sample is further normalized against the dCq of the PC as a calibrator, by subtracting the dCq pc from the dCq ₛₐₘₚₗₑ to obtain the final test result as delta delta Cq (ddCq). According to predefined cutoffs, the (semi-) quantitative ddCq value of the target can additionally be classified into positive or negative. The ddCq cutoffs were defined based on expression analysis of lung cancer samples compared to gene expression in normal lung and other normal tissues.

### Established Assay Mixes:

| Assay Mix | FAM | HEX | ATTO674N |
|---|---|---|---|
| 1 | MAGEA4 | MAGEC1 | CALM2 |
| 2 | CT83 | PRAME | HUWE1 |
| 3 | CLDN6 | MAGEA3 | MRPL19 |

ddCq result calculations

$$CombRef = ( Median Cq [ CALM 2 ] + Median Cq [ HUWE 1 ] + Median Cq [ MRPL 19 ] ) / 3$$

$${dCq}_{sample} = ( Median Cq [ {Target}_{sample} ] - [ {CombRef}_{sample} ]$$

$${dCq}_{PC} = ( Median Cq [ {Target}_{PC} ] - [ {CombRef}_{P C} ] )$$

$$ddCq = {dCq}_{sample} - {dCq}_{PC}$$

### Process Flow

FIG. 9 shows an overview of the process.

### RESULTS

TAA expression as determined by RT-qPCR was measured in 184 of 188 specimens. As some specimens' subtypes were not within our scope or too small in number to draw conclusions on that particular subtype, analysis focused on the targeted subtypes lung adenocarcinoma (LUAD), lung squamous cell carcinoma (LUSC) and large cell neuroendocrine carcinoma (LCNEC). Metastases were only obtained for LUAD in sufficient amount.

The frequency of expression of a particular TAA varies by subtype of the tumor and also between primary tumor and metastasis. While CLDN6 shows expression only in approximately one of 20 LUSC or LCNEC tumors, it still contributes to maximizing coverage across all tumors. Some TAAs appear to be more frequent in metastasis of LUAD than in their primary tumor, namely MAGEA4 and CT83. This allows to address metastases without further selection. Neuroendocrine carcinoma, although quite a different tissue, is also covered.

TABLE 3
Frequency of TAA by tumor type

| | | n= | MAGEA4 | MAGEC1 | CT83 | PRAME | CLDN6 | MAGEA3 |
|---|---|---|---|---|---|---|---|---|
| LUAD | metastases | 13 | 54% | 23% | 92% | 23% | 15% | 54% |
| | primary | 74 | 24% | 26% | 74% | 28% | 36% | 43% |
| LUSC | primary | 59 | 83% | 34% | 39% | 56% | 5% | 80% |
| LCNEC | primary | 26 | 38% | 54% | 27% | 38% | 4% | 85% |
| combined | primary | 159 | 48% | 33% | 53% | 40% | 19% | 64% |
| complete | primary + | 184 | 49% | 32% | 56% | 53% | 33% | 64% |
| cohort | metastases | | | | | | | |

TABLE 4
Coverage of tumors with six TAAs investigated. Depicted are
percentages of tumors expressing 1, 2, and up to 6 of the
TAAs simultaneously. The six selected TAAs show a broad coverage
of lung cancer tumors. While 82% of LUAD primaries are covered
by our set in this cohort, coverage auf the corresponding
metastasis is 100%. LUSC and LCNEC show coverage of 92 or
even 98%. Importantly all Subgroups show >60% coverage
with 2 targets and around 50% coverage with 3 targets.

| | | 1 | 2 | 3 | 4 | 5 | 6 |
|---|---|---|---|---|---|---|---|
| LUAD | metastases | 100% | 77% | 54% | 23% | 8% | 0% |
| | primary | 82% | 62% | 45% | 26% | 15% | 3% |
| LUSC | primary | 98% | 88% | 64% | 34% | 10% | 2% |
| LCNEC | primary | 92% | 65% | 54% | 27% | 4% | 4% |
| combined | primary | 96% | 77% | 57% | 31% | 11% | 3% |
| complete | primary + | 91% | 73% | 54% | 29% | 11% | 2% |
| cohort | metastases | | | | | | |

TABLE 5
PD-L1 expression analysis
One additional question was the availability of further confining parameters that
might allow maximizing TAA coverage in samples. To that aim, all samples were
analyzed for PD-L1 expression. PD-L1 data or mutational data (if available from the
clinical routine) were used to analyze the resulting subsets for TAA expression.

| | | PD-L1 | | Frequency of TAA: | | | | | |
|---|---|---|---|---|---|---|---|---|---|
| | | (TPS) | n= | MAGEA4 | MAGEC1 | CT83 | PRAME | CLDN6 | MAGEA3 |
| LCNEC | primary | >=0 | 26 | 38% | 54% | 27% | 38% | 4% | 85% |
| | | <10 | 19 | 47% | 47% | 26% | 37% | 0% | 84% |
| | | >=10 | 7 | 14% | 71% | 29% | 43% | 14% | 86% |
| | | <50 | 23 | 39% | 52% | 22% | 39% | 0% | 83% |
| | | >=50 | 3 | 33% | 67% | 67% | 33% | 33% | 100% |
| LUAD | primary | >=0 | 74 | 24% | 26% | 74% | 28% | 36% | 43% |
| | | <10 | 40 | 30% | 30% | 80% | 25% | 35% | 40% |
| | | >=10 | 34 | 18% | 21% | 68% | 32% | 38% | 47% |
| | | <50 | 54 | 28% | 28% | 74% | 28% | 35% | 39% |
| | | >=50 | 20 | 15% | 20% | 75% | 30% | 40% | 55% |
| | metastasis | >=0 | 13 | 54% | 23% | 92% | 23% | 15% | 54% |
| | | <10 | 6 | 50% | 33% | 83% | 33% | 33% | 67% |
| | | >=10 | 7 | 57% | 14% | 100% | 14% | 0% | 43% |
| | | <50 | 8 | 50% | 25% | 88% | 25% | 25% | 50% |
| | | >=50 | 5 | 60% | 20% | 100% | 20% | 0% | 60% |
| LUSC | primary | >=0 | 46 | 80% | 37% | 46% | 54% | 4% | 76% |
| | | <10 | 29 | 83% | 41% | 55% | 59% | 3% | 83% |
| | | >=10 | 17 | 76% | 29% | 29% | 47% | 6% | 65% |
| | | <50 | 34 | 82% | 41% | 50% | 56% | 3% | 79% |
| | | >=50 | 12 | 75% | 25% | 33% | 50% | 8% | 67% |
| | metastasis | >=0 | 1 | 100% | 0% | 0% | 100% | 0% | 0% |
| | | <10 | 1 | 100% | 0% | 0% | 100% | 0% | 0% |
| | | >=10 | 0 | −/− | −/− | −/− | −/− | −/− | −/− |
| | | <50 | 1 | 100% | 0% | 0% | 100% | 0% | 0% |
| | | >=50 | 0 | −/− | −/− | −/− | −/− | −/− | −/− |

| | | PD-L1 | | Percentage of tumors covered by at least the given number of TAA | | | | | |
|---|---|---|---|---|---|---|---|---|---|
| | | (TPS) | n= | 1 | 2 | 3 | 4 | 5 | 6 |
| LCNEC | primary | >=0 | 26 | 92% | 65% | 54% | 27% | 4% | 4% |
| | | <10 | 19 | 95% | 63% | 53% | 32% | 0% | 0% |
| | | >=10 | 7 | 86% | 71% | 57% | 14% | 14% | 14% |
| | | <50 | 23 | 91% | 65% | 52% | 26% | 0% | 0% |
| | | >=50 | 3 | 100% | 67% | 67% | 33% | 33% | 33% |
| LUAD | primary | >=0 | 74 | 82% | 62% | 45% | 26% | 15% | 3% |
| | | <10 | 40 | 83% | 60% | 43% | 30% | 20% | 5% |
| | | >=10 | 34 | 82% | 65% | 47% | 21% | 9% | 0% |
| | | <50 | 54 | 80% | 63% | 43% | 26% | 17% | 4% |
| | | >=50 | 20 | 90% | 60% | 50% | 25% | 10% | 0% |
| | metastasis | >=0 | 13 | 100% | 77% | 54% | 23% | 8% | 0% |
| | | <10 | 6 | 100% | 83% | 67% | 33% | 17% | 0% |
| | | >=10 | 7 | 100% | 71% | 43% | 14% | 0% | 0% |
| | | <50 | 8 | 100% | 75% | 50% | 25% | 13% | 0% |
| | | >=50 | 5 | 100% | 80% | 60% | 20% | 0% | 0% |
| LUSC | primary | >=0 | 46 | 98% | 85% | 65% | 37% | 11% | 2% |
| | | <10 | 29 | 97% | 90% | 76% | 48% | 10% | 3% |
| | | >=10 | 17 | 100% | 76% | 47% | 18% | 12% | 0% |
| | | <50 | 34 | 97% | 88% | 71% | 44% | 9% | 3% |
| | | >=50 | 12 | 100% | 75% | 50% | 17% | 17% | 0% |
| | metastasis | >=0 | 1 | 100% | 100% | 0% | 0% | 0% | 0% |
| | | <10 | 1 | 100% | 100% | 0% | 0% | 0% | 0% |
| | | >=10 | 0 | −/− | −/− | −/− | −/− | −/− | −/− |
| | | <50 | 1 | 100% | 100% | 0% | 0% | 0% | 0% |
| | | >=50 | 0 | −/− | −/− | −/− | −/− | −/− | −/− |

### Example 5: In Vivo Antigen-specificT Cell Expansion Induced by BNT116 in a Humanized MHC Mouse Model

De novo induction of tumor antigen-specific T cells by RNA-LPX in vivo was demonstrated with BNT116 in a humanized MHC mouse model. BNT116 comprises six RNA-LPXs, wherein the RNA in each of the RNA-LPX is a single-stranded, 5′-capped non-nucleoside-modified uridine-containing mRNA. The RNAs comprise RBL003.3 (SEQID NO: 12, encoding MAGEA3), RBL005.3 (SEQ ID NO: 4, encoding CLDN6), RBL007.2 (SEQ ID NO: 8, encoding KK-LC-1), RBL012.2 (SEQ ID NO: 20, encoding PRAME), RBLO27.2 (SEQ ID NO: 16, encoding MAGE-A4), and RBLO35.2 (SEQ ID NO: 24, encoding MAGE-C1)

A2/DR1 mice transgenic for the HLA-A*0201 and -DRB1*01 and deficient of endogenous MHC class I and II serve as a model for investigation of T cell immunogenicity on the most frequent human HLA alleles (i.e., HLA-A2.1 and HLA-DR1).

A2/DR1 mice were vaccinated IV three times with RNA-LPX encoding MAGE-A3, CLDN6, KK-LC-1, PRAME, MAGE-A4, or MAGE-C1 (RBL003.3, RBL005.3, RBL007.2, RBL012.2, RBLO27.2, or RBLO35.2 [RBL003.3: research-grade material; all other RNAs: clinical trial material], respectively), on Day 1, 8 and 15. On Day 20, splenocytes were ex vivo restimulated with peptide mixes covering the entire lengths of each BNT116 antigen, or with P2P16P17 peptides, spanning the helper epitopes P2P16. IFN-γ production was determined in an enzyme-linked immune absorbent spot (ELISpot) assay. Controls were restimulated with irrelevant human cytomegalovirus (hCMV) pp65₄₉₅₋₅₀₄ peptide. The general health and well-being of the mice were monitored with careful observation of activity, body condition, and physical abnormalities. Individual weights were taken for all mice at Day 1, 8, 15 and 20 of the experiments.

There was no test article-related mortality and no adverse effect on body weight. One mouse treated with KK-LC-1 RNA-LPX was found in bad physical condition and had to be sacrificed on Day 7. This was considered unrelated to the test item but related to bites by aggressive cage mates which led to a number of inflammatory wounds. Necropsy was also without any findings.

Vaccination against all six BNT116 antigens resulted in antigen-specific T cell immunity. The number of IFN-γ spots produced by T cells induced by MAGE-A3, PRAME and CLDN6 RNA-LPX and restimulated with cognate peptide mix was statistically significantly higher than the number of IFN-γ spots produced by T cells from the same mice when restimulated with irrelevant control peptide. The numbers of T cells secreting IFN-γ in response to KK-LC-1, MAGE-A4 and MAGE-C1 RNA-LPX was clearly increased compared to the controls but did not reach statistical significance.

IFN-γ secretion induced by restimulation of splenocytes derived from mice immunized with BNT116 with P2P16P17 peptide mix was clearly higher compared to splenocytes from the same mice restimulated with control peptide, indicating that antigen-specific CD4⁺ T cell responses against these helper epitopes were induced (FIG. 10).

T cell functionality induced by vaccination with RNA-LPXs was further demonstrated in an in vivo cytotoxicity assay, where labeled splenocytes pulsed with the known HLA-A*0201-specific peptides for MAGE-A3 serving as targets were efficiently lysed by the induced antigen-specific CD8⁺ T cells.

These results demonstrate that vaccination with clinical grade BNT116 can efficiently de novo induce antigen-specific and cytotoxic T cells against BNT116 encoded antigens.

### Example 6: Clinical Trial Evaluating the Safety, Tolerability and Preliminary Efficacy of BNT116 Alone and in Combinations in Patients with Advanced Non-Small Cell Lung Cancer

A First-in-human (FIH) trial for BNT116 is conducted aiming to establish the safety profile and a safe dose for BNT116 monotherapy as well as for BNT116 in combination with cemiplimab or in combination with docetaxel in patients with advanced or metastasized non-small cell lung cancer (NSCLC). The trial comprises several cohorts for dose confirmation in monotherapy as well as in combinations.

Arms and Interventions are as follows:

| Arm | Intervention/treatment |
|---|---|
| Experimental: Cohort 1 - | Biological: BNT116 |
| BNT116 monotherapy | intravenous injection |
| Experimental: Cohort 2 - | Biological: BNT116 |
| BNT116 + cemiplimab | intravenous injection |
| | Biological: Cemiplimab |
| | intravenous infusion |
| Experimental: Cohort 3 - | Biological: BNT116 |
| BNT116 + docetaxel | intravenous injection |
| | Drug: Docetaxel |
| | intravenous infusion |
| Experimental: Cohort 4 - | Biological: BNT116 |
| BNT116 + cemiplimab | intravenous injection |
| | Biological: Cemiplimab |
| | intravenous infusion |

Outcome Measures are as follows:

### Primary Outcome Measures:

1. Occurrence of dose-limiting toxicities (DLTs) during Cycle 1 [Time Frame: assessed during the first cycle (21 days)]
2. Occurrence of unsolicited treatment-emergent adverse events (TEAEs) reported by relationship, seriousness, and grade according to National Cancer Institute-Common Terminology Criteria for Adverse Events (NCI-CTCAE) v5.0 [Time Frame: up to 27 months]

### Secondary Outcome Measures:

1. Overall response rate (ORR) defined as the number of patients with complete response (CR) or partial response (PR) as best overall response (BOR) according to response evaluation criteria in solid tumors (RECIST) v1.1 divided by the number of patients in the efficacy analysis set [Time Frame: up to 27 months]
2. Duration of response (DoR) defined as the time from initial response until first objective tumor progression according to RECIST v1.1 [Time Frame: up to 27 months]
3. Disease control rate (DCR) defined as the number of patients with CR or PR or stable disease (SD) as BOR according to RECIST v1.1 divided by the number of patients in the efficacy analysis set [Time Frame: up to 27 months]
4. Duration of disease control defined as the time from initial detection of stable disease or response until first objective tumor progression according to RECIST v1.1 [Time Frame: up to 27 months]
5. Progression-free survival (PFS) defined as the time of first trial treatment until the first objective tumor progression according to RECIST v1.1 or death from any cause, whichever occurs first [Time Frame: up to 48 months]
6. Overall survival (OS) defined as the time of first trial treatment until death from any cause [Time Frame: up to 48 months]
   Criteria are as follows:

### Key Inclusion Criteria:

Patients must have histologically confirmed unresectable Stage Ill or metastatic Stage IV NSCLC and measurable disease by RECIST v1.1. Note: Patients in Cohort 1 do not have to have measurable disease.
Patients in Cohorts 2 and 4 must be able to tolerate additional anti-PD-1 therapy (i.e., did not permanently discontinue anti-programmed death protein 1 (PD-1)/programmed death ligand 1 (PD-L1) therapy due to toxicity) and must have recovered to stage 1 or 0 from any previous PD-1/PD-L1-related toxicity or be on a stable hormone substitute therapy.
Patients in Cohorts 2 and 3 must have an Eastern Cooperative Oncology Group performance status (ECOG-PS) 1. Patients in Cohort 1 and 4 with an ECOG-PS of 0-2 are eligible.
   Cohort-specific inclusion criteria:

### Cohort 1:

Patients' prior therapy must have included at least a PD-1/PD-L1 inhibitor and a platinum-based chemotherapy regimen as well as one other line of systemic therapy (except if a patient is not candidate for a platinum-based chemotherapy and/or PD-1/PD-L1 inhibitor and/or another line of systemic therapy).

### Cohort 2:

Patients must present with PD-L1 expression of tumor proportion score (TPS)≥50% in tumor cells (as determined locally prior to inclusion in this trial).
Patients must present with progressive disease either
   1. in the advanced or metastasized stage of NSCLC: while on a PD-1/PD-L1 inhibitor therapy or within 6 months of termination of this treatment as first-line treatment. Or
   2. be refractory to ongoing adjuvant therapy with a PD-1/PD-L1 inhibitor that has been given for at least 3 months in monotherapy (i.e., after an initial combination therapy) before being enrolled into this trial.

### Cohort 3:

Patients' prior therapy must have included at least a PD-1/PD-L1 inhibitor and a platinum-based chemotherapy regimen (except if a patient is not candidate for a platinum-based chemotherapy and/or PD-1/PD-L1 inhibitor).

### Cohort 4:

Patients' who are not candidates for chemotherapy as first-line treatment for the advanced or metastasized stage of NSCLC may be enrolled if presenting with PD-L1 expression: TPS≥1% in tumor cells.

### Key Exclusion Criteria:

Ongoing active systemic treatment against NSCLC.
Presence of a driver mutation for which approved target therapies are available.
Ongoing or recent evidence (within the last 5 years) of significant autoimmune disease that required treatment with systemic immunosuppressive treatments which may suggest risk for immune-related adverse events. Note: Patients with autoimmune-related hyperthyroidism, autoimmune-related hypothyroidism who are in remission, or on a stable dose of thyroid-replacement hormone, vitiligo, or psoriasis may be included.
Current evidence of new or growing brain or spinal metastases during screening. Patients with leptomeningeal disease are excluded. Patients with known brain or spinal metastases may be eligible if they:
   had radiotherapy or another appropriate therapy for the brain or spinal bone metastases, AND
   have no neurological symptoms that can be attributed to the current brain lesions, AND
   have stable brain or spinal disease on the computed tomography (CT) or magnetic resonance imaging (MRI) scan within 4 weeks before signing the informed consent (confirmed by stable lesions on two scans at least 4 weeks apart), AND
   do not require steroid therapy for the treatment of brain or spinal metastases within 14 d before the first dose of trial treatment. Note: Spinal bone metastases (i.e., of the vertebrae) are allowed, unless imminent fracture or cord compression is anticipated.
Systemic immune suppression:
Current use of chronic systemic steroid medication (≤5 mg/day prednisolone equivalent is allowed); patients using physiological replacement doses of prednisone for adrenal or pituitary insufficiency are eligible.
Other clinically relevant systemic immune suppression within the last 3 months before trial enrollment.
Known history of seropositivity for human immunodeficiency virus (HIV) with CD4+ T-cell (CD4+) counts <350 cells/μL and with a history of acquired immunodeficiency syndrome (AIDS)-defining opportunistic infections.
Prior splenectomy.

### Example 7: De Novo Induction of Antigen-Specific T Cells in Human HLA-Transgenic A2/DR1 Mice by BNT116 Administered within a Single Injection

For clinical development purposes, the administration of all six BNT116 products within one injection or infusion, compared to sequential injections or infusions, would be favorable. Reducing the number of injections or infusions would decrease the physical and psychological burden on the patients, and reduce the time needed for application. In Example 5, we demonstrated that administration of each of the BNT116 RNA-LPX vaccines separately in human HLA-transgenic mice (each mouse received a vaccine composed of RNA encoding one specific BNT116 antigen) prompted the de novo induction of T cells specific for the encoded BNT116 antigens. In order to test whether combination of all BNT116 products in one single injection would still be able to induce measurable immunity against all six antigens, two BNT116 mixtures for injection where prepared according to one of two processes: RNAs were first formulated as RNA-LPX, and then mixed (process 1), or RNAs were first mixed, and then formulated as RNA-LPX (process 2).

C57BL/6 A2/DR1 mice (n=6 per group) were vaccinated three times IV with a mixture of all six BNT116 RNAs (PRAME [RBL012.2], CLDN6 [RBL005.3], KK-LC-1 [RBL007.2], MAGE-3 [RBL003.3], MAGE-A4 [RBLO27.2] and MAGE-C1 [RBLO35.2]) prepared according to process 1 or 2 on Days 1, 8 and 15. The induction of antigen-specific T cells was analyzed on Day 20 by IFN-γ production of splenocytes after ex vivo restimulation with BNT116 peptide mixes, or P2P16P17 peptide mix, spanning the helper epitopes P2P16, by ELISpot. Control wells were restimulated with irrelevant human cytomegalovirus (hCMV) pp65495-504 peptide.

The general health and well-being of the mice were monitored with careful observation of activity, body condition, and physical abnormalities. Individual weights were taken for all mice at Day 1, 8, 13, 15 and 20 of the experiment. There was no test article-related mortality. One mouse in the group receiving the BNT116 mixture according to process 2 presented with reduced body weight on day 8 (84% of initial weight at treatment start), but quickly recovered within the subsequent two days.

Vaccination with either of the two BNT116 mixtures resulted in antigen-specific T cell immunity against all six antigens (FIG. 11A). Similar to the results described in Example 5, the responses against PRAME, CLDN6 and MAGE-A4 were more dominant than responses against MAGE-A3, MAGE-C1 and KK-LC-1. Although overall immune responses were very similar between the two different processes of BNT116 mixture preparation, the immune responses targeting individual antigens were stronger when induced with BNT116 mixtures produced according to process 1 (FIG. 11B). Since the administered dose, though slightly different between the two groups, can be considered quite identical, the BNT116 mixture prepared according to process 1 may be slightly superior in its potency to induce BNT116-specific T cells in this mouse model.

These results demonstrate that vaccination with a combination of all BNT116 products within one injection is well suited to de novo induce antigen-specific T cells that produce IFN-γ upon recognition of BNT116-encoded antigens.

1. A composition or medical preparation comprising at least one RNA, wherein the at least one RNA encodes the following amino acid sequences:
(i) an amino acid sequence comprising claudin 6 (CLDN6), an immunogenic variant thereof, or an immunogenic fragment of the CLDN6 or the immunogenic variant thereof;
(ii) an amino acid sequence comprising Kita-kyushu lung cancer antigen 1 (KK-LC-1), an immunogenic variant thereof, or an immunogenic fragment of the KK-LC-1 or the immunogenic variant thereof;
(iii) an amino acid sequence comprising Melanoma antigen A3 (MAGE-A3), an immunogenic variant thereof, or an immunogenic fragment of the MAGE-A3 or the immunogenic variant thereof;
(iv) an amino acid sequence comprising Melanoma antigen 4 (MAGE-A4), an immunogenic variant thereof, or an immunogenic fragment of the MAGE-A4 or the immunogenic variant thereof;
(v) an amino acid sequence comprising Preferentially Expressed Antigen In Melanoma (PRAME), an immunogenic variant thereof, or an immunogenic fragment of the PRAME or the immunogenic variant thereof; and
(vi) an amino acid sequence comprising Melanoma antigen C1 (MAGE-C1), an immunogenic variant thereof, or an immunogenic fragment of the MAGE-C1 or the immunogenic variant thereof.

2. The composition or medical preparation of claim 1, which comprises:
(i) RNA encoding an amino acid sequence comprising claudin 6 (CLDN6), an immunogenic variant thereof, or an immunogenic fragment of the CLDN6 or the immunogenic variant thereof;
(ii) RNA encoding an amino acid sequence comprising Kita-kyushu lung cancer antigen 1 (KK-LC-1), an immunogenic variant thereof, or an immunogenic fragment of the KK-LC-1 or the immunogenic variant thereof;
(iii) RNA encoding an amino acid sequence comprising Melanoma antigen A3 (MAGE-A3), an immunogenic variant thereof, or an immunogenic fragment of the MAGE-A3 or the immunogenic variant thereof;
(iv) RNA encoding an amino acid sequence comprising Melanoma antigen 4 (MAGE-A4), an immunogenic variant thereof, or an immunogenic fragment of the MAGE-A4 or the immunogenic variant thereof;
(v) RNA encoding an amino acid sequence comprising Preferentially Expressed Antigen In Melanoma (PRAME), an immunogenic variant thereof, or an immunogenic fragment of the PRAME or the immunogenic variant thereof; and
(vi) RNA encoding an amino acid sequence comprising Melanoma antigen C1 (MAGE-C1), an immunogenic variant thereof, or an immunogenic fragment of the MAGE-C1 or the immunogenic variant thereof.

3. The composition or medical preparation of claim 1 or 2, wherein each of the amino acid sequences under (i), (ii), (iii), (iv), (v), or (vi) is encoded by a separate RNA.

4. The composition or medical preparation of any one of claims 1 to 3, wherein
(i) the RNA encoding the amino acid sequence under (i) comprises the nucleotide sequence of SEQ ID NO: 3 or 4, or a nucleotide sequence having at least 99%, 98%, 97%, 96%, 95%, 90%, 85%, or 80% identity to the nucleotide sequence of SEQ ID NO: 3 or 4; and/or
(ii) the amino acid sequence under (i) comprises the amino acid sequence of SEQ ID NO: 1 or 2, or an amino acid sequence having at least 99%, 98%, 97%, 96%, 95%, 90%, 85%, or 80% identity to the amino acid sequence of SEQ ID NO: 1 or 2.

5. The composition or medical preparation of any one of claims 1 to 4, wherein
(i) the RNA encoding the amino acid sequence under (ii) comprises the nucleotide sequence of SEQ ID NO: 7 or 8, or a nucleotide sequence having at least 99%, 98%, 97%, 96%, 95%, 90%, 85%, or 80% identity to the nucleotide sequence of SEQ ID NO: 7 or 8; and/or
(ii) the amino acid sequence under (ii) comprises the amino acid sequence of SEQ ID NO: 5 or 6, or an amino acid sequence having at least 99%, 98%, 97%, 96%, 95%, 90%, 85%, or 80% identity to the amino acid sequence of SEQ ID NO: 5 or 6.

6. The composition or medical preparation of any one of claims 1 to 5, wherein
(i) the RNA encoding the amino acid sequence under (iii) comprises the nucleotide sequence of SEQ ID NO: 11 or 12, or a nucleotide sequence having at least 99%, 98%, 97%, 96%, 95%, 90%, 85%, or 80% identity to the nucleotide sequence of SEQ ID NO: 11 or 12; and/or
(ii) the amino acid sequence under (iii) comprises the amino acid sequence of SEQ ID NO: 9 or 10, or an amino acid sequence having at least 99%, 98%, 97%, 96%, 95%, 90%, 85%, or 80% identity to the amino acid sequence of SEQ ID NO: 9 or 10.

7. The composition or medical preparation of any one of claims 1 to 6, wherein
(i) the RNA encoding the amino acid sequence under (iv) comprises the nucleotide sequence of SEQ ID NO: 15 or 16, or a nucleotide sequence having at least 99%, 98%, 97%, 96%, 95%, 90%, 85%, or 80% identity to the nucleotide sequence of SEQ ID NO: 15 or 16; and/or
(ii) the amino acid sequence under (iv) comprises the amino acid sequence of SEQ ID NO: 13 or 14, or an amino acid sequence having at least 99%, 98%, 97%, 96%, 95%, 90%, 85%, or 80% identity to the amino acid sequence of SEQ ID NO: 13 or 14.

8. The composition or medical preparation of any one of claims 1 to 7, wherein
(i) the RNA encoding the amino acid sequence under (v) comprises the nucleotide sequence of SEQ ID NO: 19 or 20, or a nucleotide sequence having at least 99%, 98%, 97%, 96%, 95%, 90%, 85%, or 80% identity to the nucleotide sequence of SEQ ID NO: 19 or 20; and/or
(ii) the amino acid sequence under (v) comprises the amino acid sequence of SEQ ID NO: 17 or 18, or an amino acid sequence having at least 99%, 98%, 97%, 96%, 95%, 90%, 85%, or 80% identity to the amino acid sequence of SEQ ID NO: 17 or 18.

9. The composition or medical preparation of any one of claims 1 to 8, wherein
(i) the RNA encoding the amino acid sequence under (vi) comprises the nucleotide sequence of SEQ ID NO: 23 or 24, or a nucleotide sequence having at least 99%, 98%, 97%, 96%, 95%, 90%, 85%, or 80% identity to the nucleotide sequence of SEQ ID NO: 23 or 24; and/or
(ii) the amino acid sequence under (vi) comprises the amino acid sequence of SEQ ID NO: 21 or 22, or an amino acid sequence having at least 99%, 98%, 97%, 96%, 95%, 90%, 85%, or 80% identity to the amino acid sequence of SEQ ID NO: 21 or 22.

10. The composition or medical preparation of any one of claims 1 to 9, which comprises:
(i) RNA comprising the nucleotide sequence of SEQ ID NO: 4;
(ii) RNA comprising the nucleotide sequence of SEQ ID NO: 8;
(iii) RNA comprising the nucleotide sequence of SEQ ID NO: 12;
(iv) RNA comprising the nucleotide sequence of SEQ ID NO: 16;
(v) RNA comprising the nucleotide sequence of SEQ ID NO: 20; and
(vi) RNA comprising the nucleotide sequence of SEQ ID NO: 24.

11. The composition or medical preparation of any one of claims 1 to 10, wherein at least one amino acid sequence under (i), (ii), (iii), (iv), (v), or (vi) comprises an amino acid sequence which breaks immunological tolerance and/or at least one RNA is co-administered with RNA encoding an amino acid sequence which breaks immunological tolerance.

12. The composition or medical preparation of any one of claims 1 to 11, wherein each amino acid sequence under (i), (ii), (iii), (iv), (v), or (vi) comprises an amino acid sequence which breaks immunological tolerance and/or each RNA is co-administered with RNA encoding an amino acid sequence which breaks immunological tolerance.

13. The composition or medical preparation of claim 11 or 12, wherein the amino acid sequence which breaks immunological tolerance comprises helper epitopes, preferably tetanus toxoid-derived helper epitopes.

14. The composition or medical preparation of any one of claims 11 to 13, wherein
(i) the RNA encoding the amino acid sequence which breaks immunological tolerance comprises the nucleotide sequence of SEQ ID NO: 34, or a nucleotide sequence having at least 99%, 98%, 97%, 96%, 95%, 90%, 85%, or 80% identity to the nucleotide sequence of SEQ ID NO: 34; and/or
(ii) the amino acid sequence which breaks immunological tolerance comprises the amino acid sequence of SEQ ID NO: 33, or an amino acid sequence having at least 99%, 98%, 97%, 96%, 95%, 90%, 85%, or 80% identity to the amino acid sequence of SEQ ID NO: 33.

15. The composition or medical preparation of any one of claims 1 to 14, wherein at least one of the amino acid sequences under (i), (ii), (iii), (iv), (v), or (vi) is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

16. The composition or medical preparation of any one of claims 1 to 15, wherein each of the amino acid sequences under (i), (ii), (iii), (iv), (v), or (vi) is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

17. The composition or medical preparation of any one of claims 1 to 16, wherein at least one RNA comprises the 5′ cap m₂7,2′OGppₛp(5′)G.

18. The composition or medical preparation of any one of claims 1 to 17, wherein each RNA comprises the 5′ cap m₂7,2′-OGppₛp(5′)G.

19. The composition or medical preparation of any one of claims 1 to 18, wherein at least one RNA comprises a 5′ UTR comprising the nucleotide sequence of SEQ ID NO: 35, or a nucleotide sequence having at least 99%, 98%, 97%, 96%, 95%, 90%, 85%, or 80% identity to the nucleotide sequence of SEQ ID NO: 35.

20. The composition or medical preparation of any one of claims 1 to 19, wherein each RNA comprises a 5′ UTR comprising the nucleotide sequence of SEQ ID NO: 35, or a nucleotide sequence having at least 99%, 98%, 97%, 96%, 95%, 90%, 85%, or 80% identity to the nucleotide sequence of SEQ ID NO: 35.

21. The composition or medical preparation of any one of claims 1 to 20, wherein at least one amino acid sequence under (i), (ii), (iii), (iv), (v), or (vi) comprises an amino acid sequence enhancing antigen processing and/or presentation.

22. The composition or medical preparation of any one of claims 1 to 21, wherein each amino acid sequence under (i), (ii), (iii), (iv), (v), or (vi) comprises an amino acid sequence enhancing antigen processing and/or presentation.

23. The composition or medical preparation of claim 21 or 22, wherein the amino acid sequence enhancing antigen processing and/or presentation comprises an amino acid sequence corresponding to the transmembrane and cytoplasmic domain of a MHC molecule, preferably a MHC class I molecule.

24. The composition or medical preparation of any one of claims 21 to 23, wherein
(i) the RNA encoding the amino acid sequence enhancing antigen processing and/or presentation comprises the nucleotide sequence of SEQ ID NO: 32, or a nucleotide sequence having at least 99%, 98%, 97%, 96%, 95%, 90%, 85%, or 80% identity to the nucleotide sequence of SEQ ID NO: 32; and/or
(ii) the amino acid sequence enhancing antigen processing and/or presentation comprises the amino acid sequence of SEQ ID NO: 31, or an amino acid sequence having at least 99%, 98%, 97%, 96%, 95%, 90%, 85%, or 80% identity to the amino acid sequence of SEQ ID NO: 31.

25. The composition or medical preparation of any one of claims 1 to 24, wherein at least one RNA comprises a 3′ UTR comprising the nucleotide sequence of SEQ ID NO: 36, or a nucleotide sequence having at least 99%, 98%, 97%, 96%, 95%, 90%, 85%, or 80% identity to the nucleotide sequence of SEQ ID NO: 36.

26. The composition or medical preparation of any one of claims 1 to 25, wherein each RNA comprises a 3′ UTR comprising the nucleotide sequence of SEQ ID NO: 36, or a nucleotide sequence having at least 99%, 98%, 97%, 96%, 95%, 90%, 85%, or 80% identity to the nucleotide sequence of SEQ ID NO: 36.

27. The composition or medical preparation of any one of claims 1 to 26, wherein at least one RNA comprises a poly-A sequence.

28. The composition or medical preparation of any one of claims 1 to 27, wherein each RNA comprises a poly-A sequence.

29. The composition or medical preparation of claim 27 or 28, wherein the poly-A sequence comprises at least 100 nucleotides.

30. The composition or medical preparation of any one of claims 27 to 29, wherein the poly-A sequence comprises or consists of the nucleotide sequence of SEQ ID NO: 37.

31. The composition or medical preparation of any one of claims 1 to 30, wherein the RNA is formulated as a liquid, formulated as a solid, or a combination thereof.

32. The composition or medical preparation of any one of claims 1 to 31, wherein the RNA is formulated for injection.

33. The composition or medical preparation of any one of claims 1 to 32, wherein the RNA is formulated for intravenous administration.

34. The composition or medical preparation of any one of claims 1 to 33, wherein the RNA is formulated or is to be formulated as lipoplex particles.

35. The composition or medical preparation of any one of claims 1 to 34, wherein the RNA lipoplex particles are obtainable by mixing the RNA with liposomes.

36. The composition or medical preparation of claim 34 or 35, wherein at least one RNA encoding an amino acid sequence under (i), (ii), (iii), (iv), (v), or (vi) is co-formulated or is to be co-formulated as lipoplex particles with RNA encoding an amino acid sequence which breaks immunological tolerance.

37. The composition or medical preparation of any one of claims 34 to 36, wherein each RNA encoding an amino acid sequence under (i), (ii), (iii), (iv), (v), or (vi) is co-formulated or is to be co-formulated as lipoplex particles with RNA encoding an amino acid sequence which breaks immunological tolerance.

38. The composition or medical preparation of any one of claims 1 to 37, which is a pharmaceutical composition.

39. The composition or medical preparation of claim 38, wherein the pharmaceutical composition further comprises one or more pharmaceutically acceptable carriers, diluents and/or excipients.

40. The composition or medical preparation of any one of claims 1 to 37, wherein the medical preparation is a kit.

41. The composition or medical preparation of claim 40, wherein the RNAs are in separate vials.

42. The composition or medical preparation of claim 40 or 41, further comprising instructions for use of the composition or medical preparation for treating or preventing lung cancer.

43. The composition or medical preparation of any one of claims 1 to 42 for pharmaceutical use.

44. The composition or medical preparation of claim 43, wherein the pharmaceutical use comprises a therapeutic or prophylactic treatment of a disease or disorder.

45. The composition or medical preparation of claim 44, wherein the therapeutic or prophylactic treatment of a disease or disorder comprises treating or preventing lung cancer.

46. The composition or medical preparation of any one of claims 1 to 45, which is for administration to a human.

47. A method of treating lung cancer in a subject comprising administering at least one RNA to the subject, wherein the at least one RNA encodes the following amino acid sequences:
(i) an amino acid sequence comprising claudin 6 (CLDN6), an immunogenic variant thereof, or an immunogenic fragment of the CLDN6 or the immunogenic variant thereof;
(ii) an amino acid sequence comprising Kita-kyushu lung cancer antigen 1 (KK-LC-1), an immunogenic variant thereof, or an immunogenic fragment of the KK-LC-1 or the immunogenic variant thereof;
(iii) an amino acid sequence comprising Melanoma antigen A3 (MAGE-A3), an immunogenic variant thereof, or an immunogenic fragment of the MAGE-A3 or the immunogenic variant thereof;
(iv) an amino acid sequence comprising Melanoma antigen 4 (MAGE-A4), an immunogenic variant thereof, or an immunogenic fragment of the MAGE-A4 or the immunogenic variant thereof; and
(v) an amino acid sequence comprising Preferentially Expressed Antigen In Melanoma (PRAME), an immunogenic variant thereof, or an immunogenic fragment of the PRAME or the immunogenic variant thereof; and
(vi) an amino acid sequence comprising Melanoma antigen C1 (MAGE-C1), an immunogenic variant thereof, or an immunogenic fragment of the MAGE-C1 or the immunogenic variant thereof.

48. The method of claim 47, which comprises administering:
(i) RNA encoding an amino acid sequence comprising claudin 6 (CLDN6), an immunogenic variant thereof, or an immunogenic fragment of the CLDN6 or the immunogenic variant thereof;
(ii) RNA encoding an amino acid sequence comprising Kita-kyushu lung cancer antigen 1 (KK-LC-1), an immunogenic variant thereof, or an immunogenic fragment of the KK-LC-1 or the immunogenic variant thereof;
(iii) RNA encoding an amino acid sequence comprising Melanoma antigen A3 (MAGE-A3), an immunogenic variant thereof, or an immunogenic fragment of the MAGE-A3 or the immunogenic variant thereof;
(iv) RNA encoding an amino acid sequence comprising Melanoma antigen 4 (MAGE-A4), an immunogenic variant thereof, or an immunogenic fragment of the MAGE-A4 or the immunogenic variant thereof;
(v) RNA encoding an amino acid sequence comprising Preferentially Expressed Antigen In Melanoma (PRAME), an immunogenic variant thereof, or an immunogenic fragment of the PRAME or the immunogenic variant thereof; and
(vi) RNA encoding an amino acid sequence comprising Melanoma antigen C1 (MAGE-C1), an immunogenic variant thereof, or an immunogenic fragment of the MAGE-C1 or the immunogenic variant thereof.

49. The method of claim 47 or 48, wherein each of the amino acid sequences under (i), (ii), (iii), (iv), (v), or (vi) is encoded by a separate RNA.

50. The method of any one of claims 47 to 49, wherein
(i) the RNA encoding the amino acid sequence under (i) comprises the nucleotide sequence of SEQ ID NO: 3 or 4, or a nucleotide sequence having at least 99%, 98%, 97%, 96%, 95%, 90%, 85%, or 80% identity to the nucleotide sequence of SEQ ID NO: 3 or 4; and/or
(ii) the amino acid sequence under (i) comprises the amino acid sequence of SEQ ID NO: 1 or 2, or an amino acid sequence having at least 99%, 98%, 97%, 96%, 95%, 90%, 85%, or 80% identity to the amino acid sequence of SEQ ID NO: 1 or 2.

51. The method of any one of claims 47 to 50, wherein
(i) the RNA encoding the amino acid sequence under (ii) comprises the nucleotide sequence of SEQ ID NO: 7 or 8, or a nucleotide sequence having at least 99%, 98%, 97%, 96%, 95%, 90%, 85%, or 80% identity to the nucleotide sequence of SEQ ID NO: 7 or 8; and/or
(ii) the amino acid sequence under (ii) comprises the amino acid sequence of SEQ ID NO: 5 or 6, or an amino acid sequence having at least 99%, 98%, 97%, 96%, 95%, 90%, 85%, or 80% identity to the amino acid sequence of SEQ ID NO: 5 or 6.

52. The method of any one of claims 47 to 51, wherein
(i) the RNA encoding the amino acid sequence under (iii) comprises the nucleotide sequence of SEQ ID NO: 11 or 12, or a nucleotide sequence having at least 99%, 98%, 97%, 96%, 95%, 90%, 85%, or 80% identity to the nucleotide sequence of SEQ ID NO: 11 or 12; and/or
(ii) the amino acid sequence under (iii) comprises the amino acid sequence of SEQ ID NO: 9 or 10, or an amino acid sequence having at least 99%, 98%, 97%, 96%, 95%, 90%, 85%, or 80% identity to the amino acid sequence of SEQ ID NO: 9 or 10.

53. The method of any one of claims 47 to 52, wherein
(i) the RNA encoding the amino acid sequence under (iv) comprises the nucleotide sequence of SEQ ID NO: 15 or 16, or a nucleotide sequence having at least 99%, 98%, 97%, 96%, 95%, 90%, 85%, or 80% identity to the nucleotide sequence of SEQ ID NO: 15 or 16; and/or
(ii) the amino acid sequence under (iv) comprises the amino acid sequence of SEQ ID NO: 13 or 14, or an amino acid sequence having at least 99%, 98%, 97%, 96%, 95%, 90%, 85%, or 80% identity to the amino acid sequence of SEQ ID NO: 13 or 14.

54. The method of any one of claims 47 to 53, wherein
(i) the RNA encoding the amino acid sequence under (v) comprises the nucleotide sequence of SEQ ID NO: 19 or 20, or a nucleotide sequence having at least 99%, 98%, 97%, 96%, 95%, 90%, 85%, or 80% identity to the nucleotide sequence of SEQ ID NO: 19 or 20; and/or
(ii) the amino acid sequence under (v) comprises the amino acid sequence of SEQ ID NO: 17 or 18, or an amino acid sequence having at least 99%, 98%, 97%, 96%, 95%, 90%, 85%, or 80% identity to the amino acid sequence of SEQ ID NO: 17 or 18.

55. The method of any one of claims 47 to 54, wherein
(i) the RNA encoding the amino acid sequence under (vi) comprises the nucleotide sequence of SEQ ID NO: 23 or 24, or a nucleotide sequence having at least 99%, 98%, 97%, 96%, 95%, 90%, 85%, or 80% identity to the nucleotide sequence of SEQ ID NO: 23 or 24; and/or
(ii) the amino acid sequence under (vi) comprises the amino acid sequence of SEQ ID NO: 21 or 22, or an amino acid sequence having at least 99%, 98%, 97%, 96%, 95%, 90%, 85%, or 80% identity to the amino acid sequence of SEQ ID NO: 21 or 22.

56. The method of any one of claims 47 to 55, which comprises administering:
(i) RNA comprising the nucleotide sequence of SEQ ID NO: 4;
(ii) RNA comprising the nucleotide sequence of SEQ ID NO: 8;
(iii) RNA comprising the nucleotide sequence of SEQ ID NO: 12;
(iv) RNA comprising the nucleotide sequence of SEQ ID NO: 16;
(v) RNA comprising the nucleotide sequence of SEQ ID NO: 20; and
(vi) RNA comprising the nucleotide sequence of SEQ ID NO: 24.

57. The method of any one of claims 47 to 56, wherein at least one amino acid sequence under (i), (ii), (iii), (iv), (v), or (vi) comprises an amino acid sequence which breaks immunological tolerance and/or at least one RNA is co-administered with RNA encoding an amino acid sequence which breaks immunological tolerance.

58. The method of any one of claims 47 to 57, wherein each amino acid sequence under (i), (ii), (iii), (iv), (v), or (vi) comprises an amino acid sequence which breaks immunological tolerance and/or each RNA is co-administered with RNA encoding an amino acid sequence which breaks immunological tolerance.

59. The method of claim 57 or 58, wherein the amino acid sequence which breaks immunological tolerance comprises helper epitopes, preferably tetanus toxoid-derived helper epitopes.

60. The method of any one of claims 57 to 59, wherein
(i) the RNA encoding the amino acid sequence which breaks immunological tolerance comprises the nucleotide sequence of SEQ ID NO: 34, or a nucleotide sequence having at least 99%, 98%, 97%, 96%, 95%, 90%, 85%, or 80% identity to the nucleotide sequence of SEQ ID NO: 34; and/or
(ii) the amino acid sequence which breaks immunological tolerance comprises the amino acid sequence of SEQ ID NO: 33, or an amino acid sequence having at least 99%, 98%, 97%, 96%, 95%, 90%, 85%, or 80% identity to the amino acid sequence of SEQ ID NO: 33.

61. The method of any one of claims 47 to 60, wherein at least one of the amino acid sequences under (i), (ii), (iii), (iv), (v), or (vi) is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

62. The method of any one of claims 47 to 61, wherein each of the amino acid sequences under (i), (ii), (iii), (iv), (v), or (vi) is encoded by a coding sequence which is codon-optimized and/or the G/C content of which is increased compared to wild type coding sequence, wherein the codon-optimization and/or the increase in the G/C content preferably does not change the sequence of the encoded amino acid sequence.

63. The method of any one of claims 47 to 62, wherein at least one RNA comprises the 5′ cap m₂^{7,2′-O}Gppₛp(5′)G.

64. The method of any one of claims 47 to 63, wherein each RNA comprises the 5′ cap m₂^{7,2′-O}Gppₛp(5′)G.

65. The method of any one of claims 47 to 64, wherein at least one RNA comprises a 5′ UTR comprising the nucleotide sequence of SEQ ID NO: 35, or a nucleotide sequence having at least 99%, 98%, 97%, 96%, 95%, 90%, 85%, or 80% identity to the nucleotide sequence of SEQ ID NO: 35.

66. The method of any one of claims 47 to 65, wherein each RNA comprises a 5′ UTR comprising the nucleotide sequence of SEQ ID NO: 35, or a nucleotide sequence having at least 99%, 98%, 97%, 96%, 95%, 90%, 85%, or 80% identity to the nucleotide sequence of SEQ ID NO: 35.

67. The method of any one of claims 47 to 66, wherein at least one amino acid sequence under (i), (ii), (iii), (iv), (v), or (vi) comprises an amino acid sequence enhancing antigen processing and/or presentation.

68. The method of any one of claims 47 to 67, wherein each amino acid sequence under (i), (ii), (iii), (iv), (v), or (vi) comprises an amino acid sequence enhancing antigen processing and/or presentation.

69. The method of claim 67 or 68, wherein the amino acid sequence enhancing antigen processing and/or presentation comprises an amino acid sequence corresponding to the transmembrane and cytoplasmic domain of a MHC molecule, preferably a MHC class I molecule.

70. The method of any one of claims 67 to 69, wherein
(i) the RNA encoding the amino acid sequence enhancing antigen processing and/or presentation comprises the nucleotide sequence of SEQ ID NO: 32, or a nucleotide sequence having at least 99%, 98%, 97%, 96%, 95%, 90%, 85%, or 80% identity to the nucleotide sequence of SEQ ID NO: 32; and/or
(ii) the amino acid sequence enhancing antigen processing and/or presentation comprises the amino acid sequence of SEQ ID NO: 31, or an amino acid sequence having at least 99%, 98%, 97%, 96%, 95%, 90%, 85%, or 80% identity to the amino acid sequence of SEQ ID NO: 31.

71. The method of any one of claims 47 to 70, wherein at least one RNA comprises a 3′ UTR comprising the nucleotide sequence of SEQ ID NO: 36, or a nucleotide sequence having at least 99%, 98%, 97%, 96%, 95%, 90%, 85%, or 80% identity to the nucleotide sequence of SEQ ID NO: 36.

72. The method of any one of claims 47 to 71, wherein each RNA comprises a 3′ UTR comprising the nucleotide sequence of SEQ ID NO: 36, or a nucleotide sequence having at least 99%, 98%, 97%, 96%, 95%, 90%, 85%, or 80% identity to the nucleotide sequence of SEQ ID NO: 36.

73. The method of any one of claims 47 to 72, wherein at least one RNA comprises a poly-A sequence.

74. The method of any one of claims 47 to 73, wherein each RNA comprises a poly-A sequence.

75. The method of claim 73 or 74, wherein the poly-A sequence comprises at least 100 nucleotides.

76. The method of any one of claims 73 to 75, wherein the poly-A sequence comprises or consists of the nucleotide sequence of SEQ ID NO: 37.

77. The method of any one of claims 47 to 76, wherein the RNA is administered by injection.

78. The method of any one of claims 47 to 77, wherein the RNA is administered by intravenous administration.

79. The method of any one of claims 47 to 78, wherein the RNA is formulated as lipoplex particles.

80. The method of any one of claims 47 to 79, wherein the RNA lipoplex particles are obtainable by mixing the RNA with liposomes.

81. The method of claim 79 or 80, wherein at least one RNA encoding an amino acid sequence under (i), (ii), (iii), (iv), (v), or (vi) is co-formulated as lipoplex particles with RNA encoding an amino acid sequence which breaks immunological tolerance.

82. The method of any one of claims 79 to 81, wherein each RNA encoding an amino acid sequence under (i), (ii), (iii), (iv), (v), or (vi) is co-formulated as lipoplex particles with RNA encoding an amino acid sequence which breaks immunological tolerance.

83. The method of any one of claims 47 to 82, wherein the subject is a human.


SEQUENCE LISTING (61 sequences)

SEQ ID NO: 1 - protein, 220 aa, Homo sapiens
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 2 - protein, 295 aa, synthetic construct
  REGION 1..295: note = Vaccine sequence
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYVGGSGGGGSGGKKQYIKANSKFIGITELKKLGGGKRGGGKKMTNSVDDALINSTKIYSYFPSVISKVNQGAQGKKL

SEQ ID NO: 3 - RNA, 660 nt, Homo sapiens
  source 1..660: mol_type = other RNA
atggcctctgccggaatgcagatcctgggcgtggtgctgaccctgctgggctgggtgaatggcctggtgagctgtgccctgcccatgtggaaggtgacagccttcattggcaacagcattgtggtggcccaggtggtgtgggagggcctgtggatgagctgtgtggtgcagagcacaggccagatgcagtgcaaggtgtatgacagcctgctggccctgcctcaggacctccaggccgccagagccctgtgtgtgattgccctgctggtggccctgtttggcctgctggtgtacctggctggagccaagtgcaccacctgtgtggaggagaaggacagcaaggccagactggtgctgacctctggcattgtgtttgtgatctctggcgtgctgaccctgatccctgtgtgctggacagcccatgccatcatcagagacttctacaaccctctggtggccgaggcccagaaaagagagctgggagccagcctgtacctgggctgggccgcctctggccttcttctgctgggaggaggactgctgtgctgcacctgcccctctggcggcagccagggccccagccactacatggccagatacagcacctctgcccctgccatcagcagaggcccttctgagtaccccaccaagaactatgtg

SEQ ID NO: 4 - RNA, 1367 nt, synthetic construct
  misc_feature 1..1367: note = Vaccine sequence
  source 1..1367: mol_type = other RNA
gggcgaactagtattcttctggtccccacagactcagagagaacccgccaccatggcctctgccggaatgcagatcctgggcgtggtgctgaccctgctgggctgggtgaatggcctggtgagctgtgccctgcccatgtggaaggtgacagccttcattggcaacagcattgtggtggcccaggtggtgtgggagggcctgtggatgagctgtgtggtgcagagcacaggccagatgcagtgcaaggtgtatgacagcctgctggccctgcctcaggacctccaggccgccagagccctgtgtgtgattgccctgctggtggccctgtttggcctgctggtgtacctggctggagccaagtgcaccacctgtgtggaggagaaggacagcaaggccagactggtgctgacctctggcattgtgtttgtgatctctggcgtgctgaccctgatccctgtgtgctggacagcccatgccatcatcagagacttctacaaccctctggtggccgaggcccagaaaagagagctgggagccagcctgtacctgggctgggccgcctctggccttcttctgctgggaggaggactgctgtgctgcacctgcccctctggcggcagccagggccccagccactacatggccagatacagcacctctgcccctgccatcagcagaggcccttctgagtaccccaccaagaactatgtgggaggatccggtggtggcggcagcggcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgtgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 5 - protein, 113 aa, Homo sapiens
MNFYLLLASSILCALIVFWKYRRFQRNTGEMSSNSTALALVRPSSSGLINSNTDNNLAVYDLSRDILNNFPHSIARQKRILVNLSMVENKLVELEHTLLSKGFRGASPHRKST

SEQ ID NO: 6 - protein, 188 aa, synthetic construct
  REGION 1..188: note = Vaccine sequence
MNFYLLLASSILCALIVFWKYRRFQRNTGEMSSNSTALALVRPSSSGLINSNTDNNLAVYDLSRDILNNFPHSIARQKRILVNLSMVENKLVELEHTLLSKGFRGASPHRKSTGGSGGGGSGGKKQYIKANSKFIGITELKKLGGGKRGGGKKMTNSVDDALINSTKIYSYFPSVISKVNQGAQGKKL

SEQ ID NO: 7 - RNA, 339 nt, Homo sapiens
  source 1..339: mol_type = other RNA
atgaacttttacctgctgcttgccagcagcattctgtgcgccctgattgtgttttggaaatacagaagatttcagagaaacacaggagaaatgtcttccaattcaacagctctggctctggtgagaccttcttcttctggactgatcaattccaacacagacaacaatctggctgtgtacgatctgtccagagacattctgaacaattttcctcactcaattgcaagacagaaaagaattctggtgaatctgtcaatggtggaaaacaaactggtggaactggaacacacacttctgagcaaaggattcagaggagcttctcctcacagaaaatccaca

SEQ ID NO: 8 - RNA, 1046 nt, synthetic construct
  misc_feature 1..1046: note = Vaccine sequence
  source 1..1046: mol_type = other RNA
gggcgaactagtattcttctggtccccacagactcagagagaacccgccaccatgaacttttacctgctgcttgccagcagcattctgtgcgccctgattgtgttttggaaatacagaagatttcagagaaacacaggagaaatgtcttccaattcaacagctctggctctggtgagaccttcttcttctggactgatcaattccaacacagacaacaatctggctgtgtacgatctgtccagagacattctgaacaattttcctcactcaattgcaagacagaaaagaattctggtgaatctgtcaatggtggaaaacaaactggtggaactggaacacacacttctgagcaaaggattcagaggagcttctcctcacagaaaatccacaggaggatccggtggtggcggcagcggcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgtgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 9 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 10 - protein, 494 aa, synthetic construct
  REGION 1..494: note = Vaccine sequence
MRVMAPRTLILLLSGALALTETWAGSGGSGGGGSGGMPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEEGGSGGGGSGGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 11 - RNA, 942 nt, Homo sapiens
  source 1..942: mol_type = other RNA
atgccccttgaacagcgctcacagcactgcaaacctgaggagggccttgaagcaaggggcgaagctctggggttggtcggtgcacaagcacccgccactgaggaacaggaagccgcgtctagctcatcaaccctggttgaagtgacactgggcgaagtgcctgctgcggagagtccagaccctccccagtcccctcaaggcgcttctagcctgcctaccacgatgaactacccactgtggtcacagagctatgaggacagttccaatcaagaagaagaaggcccgtctaccttccccgatcttgagtccgagtttcaggccgctctgtcccggaaggtggcagagctcgtgcactttctcctgttgaagtatcgagcccgggagcctgtcactaaggccgaaatgctgggctctgtagtggggaattggcagtatttcttccccgtgatcttcagcaaagcctccagcagcctgcaattggtgttcggtattgaactgatggaagtagatccgattgggcatctgtacatctttgcgacatgtctgggactgtcctatgacggactgctcggggataaccagattatgccgaaagccggtctgctgatcatagttctcgccatcattgccagagagggagattgtgctccagaggagaagatctgggaggaattgtctgtgctggaggtctttgagggtagggaggacagcattctcggcgatcccaagaaactcctgacccagcactttgtccaggagaactacctcgaatacagacaggttccaggcagtgaccctgcttgctacgagttcctttggggaccccgtgcattggtagagacaagctatgtcaaagtgctgcaccatatggtgaagatatctggaggaccacacatcagttacccaccccttcatgagtgggttctgcgcgaaggggaggag

SEQ ID NO: 12 - RNA, 1967 nt, synthetic construct
  misc_feature 1..1967: note = Vaccine sequence
  source 1..1967: mol_type = other RNA
gggcgaactagtattcttctggtccccacagactcagagagaacccgccaccatgagagtgatggcccccagaaccctgatcctgctgctgtctggcgccctggccctgacagagacatgggccggaagcggcggctctggaggaggcggctccggaggcatgccccttgaacagcgctcacagcactgcaaacctgaggagggccttgaagcaaggggcgaagctctggggttggtcggtgcacaagcacccgccactgaggaacaggaagccgcgtctagctcatcaaccctggttgaagtgacactgggcgaagtgcctgctgcggagagtccagaccctccccagtcccctcaaggcgcttctagcctgcctaccacgatgaactacccactgtggtcacagagctatgaggacagttccaatcaagaagaagaaggcccgtctaccttccccgatcttgagtccgagtttcaggccgctctgtcccggaaggtggcagagctcgtgcactttctcctgttgaagtatcgagcccgggagcctgtcactaaggccgaaatgctgggctctgtagtggggaattggcagtatttcttccccgtgatcttcagcaaagcctccagcagcctgcaattggtgttcggtattgaactgatggaagtagatccgattgggcatctgtacatctttgcgacatgtctgggactgtcctatgacggactgctcggggataaccagattatgccgaaagccggtctgctgatcatagttctcgccatcattgccagagagggagattgtgctccagaggagaagatctgggaggaattgtctgtgctggaggtctttgagggtagggaggacagcattctcggcgatcccaagaaactcctgacccagcactttgtccaggagaactacctcgaatacagacaggttccaggcagtgaccctgcttgctacgagttcctttggggaccccgtgcattggtagagacaagctatgtcaaagtgctgcaccatatggtgaagatatctggaggaccacacatcagttacccaccccttcatgagtgggttctgcgcgaaggggaggagggaggatccggtggtggcggcagcggcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgggctctagcggagggggaggctctcctggcgggggatctagcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 13 - protein, 345 aa, Homo sapiens [sequence omitted]

SEQ ID NO: 14 - protein, 489 aa, synthetic construct
  REGION 1..489: note = Vaccine sequence
MLPLSVGLWVPIAQLLPALLPAALTRVIMSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPASNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGVGGSGGGGSGGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 15 - RNA, 1035 nt, Homo sapiens
  source 1..1035: mol_type = other RNA
atgctgcctctgtctgtgggcctgtgggtgccaattgcccagctgctgcctgccctgctgcctgctgccctgaccagagtgatcatgtcttctgagcagaagtctcagcactgcaagcctgaggagggagtggaggcccaggaggaggccctgggcctggtgggagcccaggccccaacaacagaggagcaggaggctgctgtgagcagcagcagccctctggtgcctggcacactggaggaggtgcctgctgctgagtctgctggacctcctcagtctcctcagggagcctctgccctgcctacaacaatcagcttcacatgctggagacagcccaatgagggcagcagcagccaggaggaggagggcccaagcacctctcctgatgccgagagcctgttcagagaggccctgagcaacaaggtggatgagctggcccactttctgctgagaaagtacagagccaaggagctggtgacaaaggctgaaatgctggaaagagtgatcaagaattacaagagatgctttcctgtgatctttggcaaagcctctgaatctctgaagatgatctttggcattgatgtgaaggaagtggaccctgccagcaacacctacaccctggtgacctgcctgggcctgagctatgatggcctgctgggcaacaatcagatctttcccaagacaggcctgctgatcattgtgctgggcacaattgccatggagggagattctgcctctgaggaggagatctgggaggagctgggagtgatgggagtgtatgatggcagagaacacacagtgtatggagaacccagaaaactgctgacccaggattgggtgcaggaaaattacctggagtacagacaggtgcctggcagcaatcctgccagatatgagttcctgtggggaccaagagctctggctgaaacatcttatgtgaaagtgctggagcatgtggtgagagtgaatgccagagtgagaattgcctacccttctctgagagaggctgctctgctggaggaggaggagggagtg

SEQ ID NO: 16 - RNA, 1952 nt, synthetic construct
  misc_feature 1..1952: note = Vaccine sequence
  source 1..1952: mol_type = other RNA
gggcgaactagtattcttctggtccccacagactcagagagaacccgccaccatgctgcctctgtctgtgggcctgtgggtgccaattgcccagctgctgcctgccctgctgcctgctgccctgaccagagtgatcatgtcttctgagcagaagtctcagcactgcaagcctgaggagggagtggaggcccaggaggaggccctgggcctggtgggagcccaggccccaacaacagaggagcaggaggctgctgtgagcagcagcagccctctggtgcctggcacactggaggaggtgcctgctgctgagtctgctggacctcctcagtctcctcagggagcctctgccctgcctacaacaatcagcttcacatgctggagacagcccaatgagggcagcagcagccaggaggaggagggcccaagcacctctcctgatgccgagagcctgttcagagaggccctgagcaacaaggtggatgagctggcccactttctgctgagaaagtacagagccaaggagctggtgacaaaggctgaaatgctggaaagagtgatcaagaattacaagagatgctttcctgtgatctttggcaaagcctctgaatctctgaagatgatctttggcattgatgtgaaggaagtggaccctgccagcaacacctacaccctggtgacctgcctgggcctgagctatgatggcctgctgggcaacaatcagatctttcccaagacaggcctgctgatcattgtgctgggcacaattgccatggagggagattctgcctctgaggaggagatctgggaggagctgggagtgatgggagtgtatgatggcagagaacacacagtgtatggagaacccagaaaactgctgacccaggattgggtgcaggaaaattacctggagtacagacaggtgcctggcagcaatcctgccagatatgagttcctgtggggaccaagagctctggctgaaacatcttatgtgaaagtgctggagcatgtggtgagagtgaatgccagagtgagaattgcctacccttctctgagagaggctgctctgctggaggaggaggagggagtgggaggatccggtggtggcggcagcggcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgggctctagcggagggggaggctctcctggcgggggatctagcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 17 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 18 - protein, 689 aa, synthetic construct
  REGION 1..689: note = Vaccine sequence
MRVMAPRTLILLLSGALALTETWAGSGGSGGGGSGGMERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPNGGSGGGGSGGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 19 - RNA, 1527 nt, Homo sapiens
  source 1..1527: mol_type = other RNA
atggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctgttcccgccactgttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaggaacagtatatcgcccagttcacctctcagttcctcagtctgcagtgcctccaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacaaccttaagcttctacgggaattccatctccatatctgccttgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaac

SEQ ID NO: 20 - RNA, 2552 nt, synthetic construct
  misc_feature 1..2552: note = Vaccine sequence
  source 1..2552: mol_type = other RNA
gggcgaactagtattcttctggtccccacagactcagagagaacccgccaccatgagagtgatggcccccagaaccctgatcctgctgctgtctggcgccctggccctgacagagacatgggccggaagcggcggctctggaggaggcggctccggaggcatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctgttcccgccactgttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaggaacagtatatcgcccagttcacctctcagttcctcagtctgcagtgcctccaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacaaccttaagcttctacgggaattccatctccatatctgccttgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaacggaggatccggtggtggcggcagcggcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgggctctagcggagggggaggctctcctggcgggggatctagcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 21 - protein, 209 aa, Homo sapiens
MLTNVISRYTGYFPVIFRKAREFIEILFGISLREVDPDDSYVFVNTLDLTSEGCLSDEQGMSQNRLLILILSIIFIKGTYASEEVIWDVLSGIGVRAGREHFAFGEPRELLTKVWVQEHYLEYREVPNSSPPRYEFLWGPRAHSEVIKRKVVEFLAMLKNTVPITFPSSYKDALKDVEERAQAIIDTTDDSTATESASSSVMSPSFSSE

SEQ ID NO: 22 - protein, 389 aa, synthetic construct
  REGION 1..389: note = Vaccine sequence
MRVMAPRTLILLLSGALALTETWAGSGGSGGGGSGGMLTNVISRYTGYFPVIFRKAREFIEILFGISLREVDPDDSYVFVNTLDLTSEGCLSDEQGMSQNRLLILILSIIFIKGTYASEEVIWDVLSGIGVRAGREHFAFGEPRELLTKVWVQEHYLEYREVPNSSPPRYEFLWGPRAHSEVIKRKVVEFLAMLKNTVPITFPSSYKDALKDVEERAQAIIDTTDDSTATESASSSVMSPSFSSEGGSGGGGSGGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 23 - RNA, 627 nt, Homo sapiens
  source 1..627: mol_type = other RNA
atgctgacgaatgtcatcagcaggtacacgggctactttcctgtgatcttcaggaaagcccgtgagttcatagagatactttttggcatttccctgagagaagtggaccctgatgactcctatgtctttgtaaacacattagacctcacctctgaggggtgtctgagtgatgagcagggcatgtcccagaaccgcctcctgattcttattctgagtatcatcttcataaagggcacctatgcctctgaggaggtcatctgggatgtgctgagtggaataggggtgcgtgctgggagggagcactttgcctttggggagcccagggagctcctcactaaagtttgggtgcaggaacattacctagagtaccgggaggtgcccaattcttctcctcctcgttacgaattcctgtggggtccaagagctcattcagaagtcattaagaggaaagtagtagagtttttggccatgctaaagaataccgtccctattacctttccatcctcttacaaggatgctttgaaagatgtggaggagagagcccaggccataattgacaccacagatgattcgactgccacagaaagtgcaagctccagtgtcatgtcccccagcttttcttctgag

SEQ ID NO: 24 - RNA, 1652 nt, synthetic construct
  misc_feature 1..1652: note = Vaccine sequence
  source 1..1652: mol_type = other RNA
gggcgaactagtattcttctggtccccacagactcagagagaacccgccaccatgagagtgatggcccccagaaccctgatcctgctgctgtctggcgccctggccctgacagagacatgggccggaagcggcggctctggaggaggcggctccggaggcatgctgacgaatgtcatcagcaggtacacgggctactttcctgtgatcttcaggaaagcccgtgagttcatagagatactttttggcatttccctgagagaagtggaccctgatgactcctatgtctttgtaaacacattagacctcacctctgaggggtgtctgagtgatgagcagggcatgtcccagaaccgcctcctgattcttattctgagtatcatcttcataaagggcacctatgcctctgaggaggtcatctgggatgtgctgagtggaataggggtgcgtgctgggagggagcactttgcctttggggagcccagggagctcctcactaaagtttgggtgcaggaacattacctagagtaccgggaggtgcccaattcttctcctcctcgttacgaattcctgtggggtccaagagctcattcagaagtcattaagaggaaagtagtagagtttttggccatgctaaagaataccgtccctattacctttccatcctcttacaaggatgctttgaaagatgtggaggagagagcccaggccataattgacaccacagatgattcgactgccacagaaagtgcaagctccagtgtcatgtcccccagcttttcttctgagggaggatccggtggtggcggcagcggcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgggctctagcggagggggaggctctcctggcgggggatctagcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 25 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 26 - protein, 360 aa, synthetic construct
  REGION 1..360: note = Vaccine sequence
MRVMAPRTLILLLSGALALTETWAGSGGSGGGGSGGMQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRRGGSGGGGSGGKKQYIKANSKFIGITELKKLGGGKRGGGKKMTNSVDDALINSTKIYSYFPSVISKVNQGAQGKKLGSSGGGGSPGGGSSIVGIVAGLAVLAVVVIGAVVATVMCRRKSSGGKGGSYSQAASSDSAQGSDVSLTA

SEQ ID NO: 27 - RNA, 540 nt, Homo sapiens
  source 1..540: mol_type = other RNA
atgcaggccgagggcagaggaacaggcggcagcacaggcgacgcagatggaccaggcggccctggaatccctgatggcccaggcggcaatgctgggggaccaggagaagctggcgccacaggcgggagaggacctagaggagctggagccgctagagcttctggacctgggggaggcgcccctagaggaccacatggaggcgctgccagcggcctgaatggctgctgcagatgcggcgccagaggccctgagagccggctgctggaattctacctggccatgcccttcgccacccccatggaagccgagctggccagaagatccctggctcaggacgctcctcctctgcctgtgcccggcgtgctgctgaaagaattcaccgtgtccggcaacatcctgaccatcagactgacagccgccgatcacagacagctccagctgagcatcagctcttgcctgcagcagctgagcctgctgatgtggatcacccagtgctttctgcccgtgttcctggcccagccacccagcggacagagaagg

SEQ ID NO: 28 - RNA, 1565 nt, synthetic construct
  misc_feature 1..1565: note = Vaccine sequence
  source 1..1565: mol_type = other RNA
gggcgaactagtattcttctggtccccacagactcagagagaacccgccaccatgagagtgatggcccccagaaccctgatcctgctgctgtctggcgccctggccctgacagagacatgggccggaagcggcggctctggaggaggcggctccggaggcatgcaggccgagggcagaggaacaggcggcagcacaggcgacgcagatggaccaggcggccctggaatccctgatggcccaggcggcaatgctgggggaccaggagaagctggcgccacaggcgggagaggacctagaggagctggagccgctagagcttctggacctgggggaggcgcccctagaggaccacatggaggcgctgccagcggcctgaatggctgctgcagatgcggcgccagaggccctgagagccggctgctggaattctacctggccatgcccttcgccacccccatggaagccgagctggccagaagatccctggctcaggacgctcctcctctgcctgtgcccggcgtgctgctgaaagaattcaccgtgtccggcaacatcctgaccatcagactgacagccgccgatcacagacagctccagctgagcatcagctcttgcctgcagcagctgagcctgctgatgtggatcacccagtgctttctgcccgtgttcctggcccagccacccagcggacagagaaggggaggatccggtggtggcggcagcggcggcaagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactgggctctagcggagggggaggctctcctggcgggggatctagcatcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcctagtaactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccgagacctggtccagagtcgctagccgcgtcgctaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 29 - protein, 26 aa, synthetic construct
  REGION 1..26: note = Secretory sequence
MRVMAPRTLILLLSGALALTETWAGS

SEQ ID NO: 30 - RNA, 78 nt, synthetic construct
  misc_feature 1..78: note = Secretory sequence
  source 1..78: mol_type = other RNA
atgagagtgatggcccccagaaccctgatcctgctgctgtctggcgccctggccctgacagagacatgggccggaagc

SEQ ID NO: 31 - protein, 55 aa, synthetic construct
  REGION 1..55: note = MITD
IVGIVAGLAVLAVVVIGAVVATVMCRRKSSGGKGGSYSQAASSDSAQGSDVSLTA

SEQ ID NO: 32 - RNA, 165 nt, synthetic construct
  misc_feature 1..165: note = MITD
  source 1..165: mol_type = other RNA
atcgtgggaattgtggcaggactggcagtgctggccgtggtggtgatcggagccgtggtggctaccgtgatgtgcagacggaagtccagcggaggcaagggcggcagctacagccaggccgccagctctgatagcgcccagggcagcgacgtgtcactgacagcc

SEQ ID NO: 33 - protein, 65 aa, synthetic construct
  REGION 1..65: note = P2P16
KKQYIKANSKFIGITELKKLGGGKRGGGKKMTNSVDDALINSTKIYSYFPSVISKVNQGAQGKKL

SEQ ID NO: 34 - RNA, 195 nt, synthetic construct
  misc_feature 1..195: note = P2P16
  source 1..195: mol_type = other RNA
aagaagcagtacatcaaggccaacagcaagttcatcggcatcaccgagctgaagaagctgggagggggcaaacggggaggcggcaaaaagatgaccaacagcgtggacgacgccctgatcaacagcaccaagatctacagctacttccccagcgtgatcagcaaagtgaaccagggcgctcagggcaagaaactg

SEQ ID NO: 35 - RNA, 47 nt, synthetic construct
  misc_feature 1..47: note = 5'-UTR
  source 1..47: mol_type = other RNA
aactagtattcttctggtccccacagactcagagagaacccgccacc

SEQ ID NO: 36 - RNA, 278 nt, synthetic construct
  misc_feature 1..278: note = 3'-UTR
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 37 - RNA, 110 nt, synthetic construct
  misc_feature 1..110: note = A30L70
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 38 - protein, 15 aa, synthetic construct
  REGION 1..15: note = p2
QYIKANSKFIGITEL

SEQ ID NO: 39 - protein, 32 aa, synthetic construct
  REGION 1..32: note = p16
MTNSVDDALINSTKIYSYFPSVISKVNQGAQG

SEQ ID NO: 40 - protein, 10 aa, synthetic construct
  REGION 1..10: note = Linker
GGSGGGGSGG

SEQ ID NO: 41 - protein, 14 aa, synthetic construct
  REGION 1..14: note = Linker
GSSGGGGSPGGGSS

SEQ ID NO: 42 - DNA, 21 bp, synthetic construct
  misc_feature 1..21: note = Primer
  source 1..21: mol_type = other DNA
actcggcctaggaatttccct

SEQ ID NO: 43 - DNA, 17 bp, synthetic construct
  misc_feature 1..17: note = Primer
  source 1..17: mol_type = other DNA
cagaggccatggcgagg

SEQ ID NO: 44 - DNA, 22 bp, synthetic construct
  misc_feature 1..22: note = Primer
  source 1..22: mol_type = other DNA
gtgtgccttgattgtcttctgg

SEQ ID NO: 45 - DNA, 19 bp, synthetic construct
  misc_feature 1..19: note = Primer
  source 1..19: mol_type = other DNA
cctggctattgagtgtggg

SEQ ID NO: 46 - DNA, 19 bp, synthetic construct
  misc_feature 1..19: note = Primer
  source 1..19: mol_type = other DNA
cggtgaggaggcaaggttc

SEQ ID NO: 47 - DNA, 21 bp, synthetic construct
  misc_feature 1..21: note = Primer
  source 1..21: mol_type = other DNA
aatggagacccactggcagat

SEQ ID NO: 48 - DNA, 22 bp, synthetic construct
  misc_feature 1..22: note = Primer
  source 1..22: mol_type = other DNA
gcaccaaaggagaagatctgcc

SEQ ID NO: 49 - DNA, 21 bp, synthetic construct
  misc_feature 1..21: note = Primer
  source 1..21: mol_type = other DNA
tgtcaaggccttcctcaggct

SEQ ID NO: 50 - DNA, 23 bp, synthetic construct
  misc_feature 1..23: note = Primer
  source 1..23: mol_type = other DNA
gacctatccagtcttcaaggtgc

SEQ ID NO: 51 - DNA, 20 bp, synthetic construct
  misc_feature 1..20: note = Primer
  source 1..20: mol_type = other DNA
agggggtttgtcttctggga

SEQ ID NO: 52 - DNA, 18 bp, synthetic construct
  misc_feature 1..18: note = Primer
  source 1..18: mol_type = other DNA
cgtgccaggggtgcttct

SEQ ID NO: 53 - DNA, 22 bp, synthetic construct
  misc_feature 1..22: note = Primer
  source 1..22: mol_type = other DNA
tctgcagcagtcagtcggatag

SEQ ID NO: 54 - DNA, 21 bp, synthetic construct
  misc_feature 1..21: note = Primer
  source 1..21: mol_type = other DNA
gcagtatatcgcccagttcac

SEQ ID NO: 55 - DNA, 20 bp, synthetic construct
  misc_feature 1..20: note = Primer
  source 1..20: mol_type = other DNA
ggttcatcacgtgcctgagc

SEQ ID NO: 56 - DNA, 21 bp, synthetic construct
  misc_feature 1..21: note = Primer
  source 1..21: mol_type = other DNA
tgacactggcaaaacaatgca

SEQ ID NO: 57 - DNA, 21 bp, synthetic construct
  misc_feature 1..21: note = Primer
  source 1..21: mol_type = other DNA
ggtccttttcaccagcaagct

SEQ ID NO: 58 - DNA, 20 bp, synthetic construct
  misc_feature 1..20: note = Primer
  source 1..20: mol_type = other DNA
agcccagctgcagagaaagt

SEQ ID NO: 59 - DNA, 20 bp, synthetic construct
  misc_feature 1..20: note = Primer
  source 1..20: mol_type = other DNA
ggatgatggcactgaactcc

SEQ ID NO: 60 - DNA, 22 bp, synthetic construct
  misc_feature 1..22: note = Primer
  source 1..22: mol_type = other DNA
gagccaagagtgaagaacagtc

SEQ ID NO: 61 - DNA, 23 bp, synthetic construct
  misc_feature 1..23: note = Primer
  source 1..23: mol_type = other DNA
gctccccaccatattctgaatct